(19) United States
(12) Patent Application Publication
(10) Pub. No.: US 2003/0082174 A1
(43) Pub. Date: May 1, 2003

(54) Novel proteins and nucleic acids encoding same

(76) Inventors: Muralidhara Padigaru, Branford, CT (US); Kimberly A. Spytek, New Haven, CT (US); Kumud Majumder, Stamford, CT (US); Velizar T. Tchernev, Branford, CT (US); William M. Grosse, Branford, CT (US); Edward S. Szekeres JR., Branford, CT (US); John P. Alsobrook II, Madison, CT (US); Catherine E. Burgess, Wethersfield, CT (US); Richard A. Shimkets, West Haven, CT (US); Raymond J. Taupier JR., East Haven, CT (US); Stacie J. Casman, North Haven, CT (US); Esha A. Gangolli, Madison, CT (US); John R. MacDougall, Hamden, CT (US); David J. Stone, Guilford, CT (US); Glennda Smithson, Guilford, CT (US)

Correspondence Address: Ivor R. Elrifi, Esq., MINTZ, LEVIN, COHN, FERRIS,, GLOVSKY AND POPEO, P.C., One Financial Center, Boston, MA, 02111, US

(21) Appl. No.: 09/864,029

(22) Filed: May 23, 2001

Related U.S. Application Data
(60) Provisional application No. 60/207,020, filed on May 25, 2000.
(60) Provisional application No. 60/219,786, filed on Jul. 19, 2000.
(60) Provisional application No. 60/220,593, filed on Jul. 25, 2000.
(60) Provisional application No. 60/239,542, filed on Oct. 10, 2000.
(60) Provisional application No. 60/275,590, filed on Mar. 13, 2001.
(60) Provisional application No. 60/256,402, filed on Dec. 18, 2000.
(60) Provisional application No. 60/274,809, filed on Mar. 9, 2001.
(60) Provisional application No. 60/206,757, filed on May 24, 2000.
(60) Provisional application No. 60/271,645, filed on Feb. 26, 2001.
(60) Provisional application No. 60/214,372, filed on Jun. 28, 2000.

(51) Int. Cl. A61K 39/395; C07H 21/04; C12P 21/02; C12N 5/06; C07K 14/705; C07K 16/28

(52) U.S. Cl. 424/143.1; 530/350; 435/69.1; 435/325; 435/320.1; 536/23.5

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. Pat. No. 60/207020, filed May 25, 2000; U.S. Pat. No. 60/219,786, filed Jul. 19, 2000; U.S. Pat. No. 60/220,593, filed Jul. 25, 2000; U.S. Pat. No. 60/239,542, filed Oct. 10, 2000; U.S. Pat. No. 60/275,590, filed Mar. 13, 2001; U.S. Pat. No. 60/256,402, filed Dec. 18, 2000; U.S. Pat. No. 60/274,809, filed Mar. 9, 2001; U.S. Pat. No. 60/206,757, filed May 24, 2000; U.S. Pat. No. 60/271,645, filed Feb. 26, 2001; and U.S. Pat. No. 60/214,372, filed Jun. 28, 2000, each of which is incorporated by reference in its entirety.

### TECHNICAL FIELD OF THE INVENTION

The invention generally relates to nucleic acids and polypeptides encoded therefrom.

### BACKGROUND OF THE INVENTION

Within the animal kingdom, odor detection is a universal tool used for social interaction, predation, and reproduction. Chemosensitivity in vertebrates is modulated by bipolar sensory neurons located in the olfactory epithelium, which extend a single, highly arborized dendrite into the mucosa while projecting axons to relay neurons within the olfactory bulb. The many ciliae on the neurons bear odorant (or olfactory) receptors (ORs), which cause depolarization and formation of action potentials upon contact with specific odorants. ORs may also function as axonal guidance molecules, a necessary function as the sensory neurons are normally renewed continuously through adulthood by underlying populations of basal cells.

The mammalian olfactory system is able to distinguish several thousand odorant molecules. Odorant receptors are believed to be encoded by an extremely large subfamily of G protein-coupled receptors. These receptors share a 7-transmembrane domain structure with many neurotransmitter and hormone receptors and are likely to underlie the recognition and G-protein-mediated transduction of odorant signals and possibly other chemosensing responses as well. The genes encoding these receptors are devoid of introns within their coding regions. Schurmans and co-workers cloned a member of this family of genes, OLFR1, from a genomic library by cross-hybridization with a gene fragment obtained by PCR. See Schurmans et al., Cytogenet. Cell Genet., 1993, 63(3):200. By isotopic in situ hybridization, they mapped the gene to 17p13-p12 with a peak at band 17p13. A minor peak was detected on chromosome 3, with a maximum in the region 3q13-q21. After MspI digestion, a restriction fragment length polymorphism (RFLP) was demonstrated. Using this in a study of 3 CEPH pedigrees, they demonstrated linkage with D17S126 at 17pter-p12; maximum lod=3.6 at theta=0.0. Used as a probe on Southern blots under moderately stringent conditions, the cDNA hybridized to at least 3 closely related genes. Ben-Arie and colleagues cloned 16 human OLFR genes, all from 17p13.3. See Ben-Arie et al., Hum. Mol. Genet., 1994, 3(2):229. The intronless coding regions are mapped to a 350-kb contiguous cluster, with an average intergenic separation of 15 kb. The OLFR genes in the cluster belong to 4 different gene subfamilies, displaying as much sequence variability as any randomly selected group of OLFRs. This suggested that the cluster may be one of several copies of an ancestral OLFR gene repertoire whose existence may have predated the divergence of mammals. Localization to 17p13.3 was performed by fluorescence in situ hybridization as well as by somatic cell hybrid mapping.

Previously, OR genes cloned in different species were from disparate locations in the respective genomes. The human OR genes, on the other hand, lack introns and may be segregated into four different gene subfamilies, displaying great sequence variability. These genes are primarily expressed in olfactory epithelium, but may be found in other chemoresponsive cells and tissues as well.

Blache and co-workers used polymerase chain reaction (PCR) to clone an intronless cDNA encoding a new member (named OL2) of the G protein-coupled receptor superfamily. See Blache et al., Biochem. Biophys. Res. Commun., 1998, 242(3):669. The coding region of the rat OL2 receptor gene predicts a seven transmembrane domain receptor of 315 amino acids. OL2 has 46.4 percent amino acid identity with OL1, an olfactory receptor expressed in the developing rat heart, and slightly lower percent identities with several other olfactory receptors. PCR analysis reveals that the transcript is present mainly in the rat spleen and in a mouse insulin-secreting cell line (MIN6). No correlation was found between the tissue distribution of OL2 and that of the olfaction-related GTP-binding protein Golf alpha subunit. These findings suggest a role for this new hypothetical G-protein coupled receptor and for its still unknown ligand in the spleen and in the insulin-secreting beta cells.

Olfactory loss may be induced by trauma or by neoplastic growths in the olfactory neuroepithelium. There is currently no treatment available that effectively restores olfaction in the case of sensorineural olfactory losses. See Harrison's Principles of Internal Medicine, 14^{th} Ed., Fauci, AS et al. (eds.), McGraw-Hill, New York, 1998, 173. There thus remains a need for effective treatment to restore olfaction in pathologies related to neural olfactory loss.

The invention generally relates to nucleic acids and polypeptides. More particularly, the invention relates to nucleic acids encoding novel G-protein coupled receptor (GPCR) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as GPCRX, nucleic acids and polypeptides. These nucleic acids and polypeptides, as well as derivatives, homologs, analogs and fragments thereof, will hereinafter be collectively designated as “GPCRX” nucleic acid or polypeptide sequences.

In one aspect, the invention provides an isolated GPCRX nucleic acid molecule encoding a GPCRX polypeptide that includes a nucleic acid sequence that has identity to the nucleic acids disclosed in SEQ ID NOS:2n-1, wherein n is an integer between 1-13. In some embodiments, the GPCRX nucleic acid molecule will hybridize under stringent conditions to a nucleic acid sequence complementary to a nucleic acid molecule that includes a protein-coding sequence of a GPCRX nucleic acid sequence. The invention also includes an isolated nucleic acid that encodes a GPCRX polypeptide, or a fragment, homolog, analog or derivative thereof. For example, the nucleic acid can encode a polypeptide at least 80% identical to a polypeptide comprising the amino acid sequences of SEQ ID NOS:2n, wherein n is an integer between 1-13. The nucleic acid can be, for example, a genomic DNA fragment or a cDNA molecule that includes the nucleic acid sequence of any of SEQ ID NOS:2n-1, wherein n is an integer between 1-13.

Also included in the invention is an oligonucleotide, e.g., an oligonucleotide which includes at least 6 contiguous nucleotides of a GPCRX nucleic acid (e.g., SEQ ID NOS:2n-1, wherein n is an integer between 1-13) or a complement of said oligonucleotide.

Also included in the invention are substantially purified GPCRX polypeptides (SEQ ID NOS:2n, wherein n is an integer between 1-13). In certain embodiments, the GPCRX polypeptides include an amino acid sequence that is substantially identical to the amino acid sequence of a human GPCRX polypeptide.

The invention also features antibodies that immunoselectively bind to GPCRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a GPCRX nucleic acid, a GPCRX polypeptide, or an antibody specific for a GPCRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a GPCRX nucleic acid, under conditions allowing for expression of the GPCRX polypeptide encoded by the DNA. If desired, the GPCRX polypeptide can then be recovered.

In another aspect, the invention includes a method of detecting the presence of a GPCRX polypeptide in a sample. In the method, a sample is contacted with a compound that selectively binds to the polypeptide under conditions allowing for formation of a complex between the polypeptide and the compound. The complex is detected, if present, thereby identifying the GPCRX polypeptide within the sample.

The invention also includes methods to identify specific cell or tissue types based on their expression of a GPCRX.

Also included in the invention is a method of detecting the presence of a GPCRX nucleic acid molecule in a sample by contacting the sample with a GPCRX nucleic acid probe or primer, and detecting whether the nucleic acid probe or primer bound to a GPCRX nucleic acid molecule in the sample.

In a further aspect, the invention provides a method for modulating the activity of a GPCRX polypeptide by contacting a cell sample that includes the GPCRX polypeptide with a compound that binds to the GPCRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a GPCRX nucleic acid, a GPCRX polypeptide, or a GPCRX-specific antibody, or biologically-active derivatives or fragments thereof.

For example, the compositions of the present invention will have efficacy for treatment of patients suffering from: developmental diseases, MHCII and III diseases (immune diseases), taste and scent detectability Disorders, Burkitt's lymphoma, corticoneurogenic disease, signal transduction pathway disorders, Retinal diseases including those involving photoreception, Cell growth rate disorders; cell shape disorders, feeding disorders; control of feeding; potential obesity due to over-eating; potential disorders due to starvation (lack of appetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome and/or other pathologies and disorders of the like.

The polypeptides can be used as immunogens to produce antibodies specific for the invention, and as vaccines. They can also be used to screen for potential agonist and antagonist compounds. For example, a cDNA encoding GPCRX may be useful in gene therapy, and GPCRX may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a GPCRX polypeptide and determining if the test compound binds to said GPCRX polypeptide. Binding of the test compound to the GPCRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a GPCRX nucleic acid. Expression or activity of GPCRX polypeptide is then measured in the test animal, as is expression or activity of the protein in a control animal which recombinantly-expresses GPCRX polypeptide and is not at increased risk for the disorder or syndrome. Next, the expression of GPCRX polypeptide in both the test animal and the control animal is compared. A change in the activity of GPCRX polypeptide in the test animal relative to the control animal indicates the test compound is a modulator of latency of the disorder or syndrome.

In yet another aspect, the invention includes a method for determining the presence of or predisposition to a disease associated with altered levels of a GPCRX polypeptide, a GPCRX nucleic acid, or both, in a subject (e.g., a human subject). The method includes measuring the amount of the GPCRX polypeptide in a test sample from the subject and comparing the amount of the polypeptide in the test sample to the amount of the GPCRX polypeptide present in a control sample. An alteration in the level of the GPCRX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a GPCRX polypeptide, a GPCRX nucleic acid, or a GPCRX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

Olfactory receptors (ORs) are the largest family of G-protein-coupled receptors (GPCRs) and belong to the first family (Class A) of GPCRs, along with catecholamine receptors and opsins. The OR family contains over 1,000 members that traverse the phylogenetic spectrum from C. elegans to mammals. ORs most likely emerged from prototypic GPCRs several times independently, extending the structural diversity necessary both within and between species in order to differentiate the multitude of ligands. Individual olfactory sensory neurons are predicted to express a single, or at most a few, ORs. All ORs are believed to contain seven α-helices separated by three extracellular and three cytoplasmic loops, with an extracellular amino-terminus and a cytoplasmic carboxy-terminus. The pocket of OR ligand binding is expected to be between the second and sixth transmembrane domains of the proteins. Overall amino acid sequence identity within the mammalian OR family ranges from 45% to >80%, and genes greater than 80% identical to one another at the amino acid level are considered to belong to the same subfamily.

Since the first ORs were cloned in 1991, outstanding progress has been made into their mechanisms of action and potential dysregulation during disease and disorder. It is understood that some human diseases result from rare mutations within GPCRs. Drug discovery avenues could be used to produce highly specific compounds on the basis of minute structural differences of OR subtypes, which are now being appreciated with in vivo manipulation of OR levels in transgenic and knock-out animals. Furthermore, due to the intracellular homogeneity and ligand specificity of ORs, renewal of specific odorant-sensing neurons lost in disease or disorder is possible by the introduction of individual ORs into basal cells. Additionally, new therapeutic strategies may be elucidated by further study of so-called orphan receptors, whose ligand(s) remain to be discovered.

OR proteins bind odorant ligands and transmit a G-protein-mediated intracellular signal, resulting in generation of an action potential. The accumulation of DNA sequences of hundreds of OR genes provides an opportunity to predict features related to their structure, function and evolutionary diversification. See Pilpel Y, et.al., Essays Biochem 1998;33:93-104. The OR repertoire has evolved a variable ligand-binding site that ascertains recognition of multiple odorants, coupled to constant regions that mediate the cAMP-mediated signal transduction. The cellular second messenger underlies the responses to diverse odorants through the direct gating of olfactory-specific cation channels. This situation necessitates a mechanism of cellular exclusion, whereby each sensory neuron expresses only one receptor type, which in turn influences axonal projections. A ‘synaptic image’ of the OR repertoire thus encodes the detected odorant in the central nervous system.

The ability to distinguish different odors depends on a large number of different odorant receptors (ORs). ORs are expressed by nasal olfactory sensory neurons, and each neuron expresses only 1 allele of a single OR gene. In the nose, different sets of ORs are expressed in distinct spatial zones. Neurons that express the same OR gene are located in the same zone; however, in that zone they are randomly interspersed with neurons expressing other ORs. When the cell chooses an OR gene for expression, it may be restricted to a specific zonal gene set, but it may select from that set by a stochastic mechanism. Proposed models of OR gene choice fall into 2 classes: locus-dependent and locus-independent. Locus-dependent models posit that OR genes are clustered in the genome, perhaps with members of different zonal gene sets clustered at distinct loci. In contrast, locus-independent models do not require that OR genes be clustered.

OR genes have been mapped to 11 different regions on 7 chromosomes. These loci lie within paralogous chromosomal regions that appear to have arisen by duplications of large chromosomal domains followed by extensive gene duplication and divergence. Studies have shown that OR genes expressed in the same zone map to numerous loci; moreover, a single locus can contain genes expressed in different zones. These findings raised the possibility that OR gene choice is locus-independent or involved consecutive stochastic choices.

Issel-Tarver and Rine (1996) characterized 4 members of the canine olfactory receptor gene family. The 4 subfamilies comprised genes expressed exclusively in olfactory epithelium. Analysis of large DNA fragments using Southern blots of pulsed field gels indicated that subfamily members were clustered together, and that two of the subfamilies were closely linked in the dog genome. Analysis of the four olfactory receptor gene subfamilies in 26 breeds of dog provided evidence that the number of genes per subfamily was stable in spite of differential selection on the basis of olfactory acuity in scent hounds, sight hounds, and toy breeds.

Issel-Tarver and Rine (1997) performed a comparative study of four subfamilies of olfactory receptor genes first identified in the dog to assess changes in the gene family during mammalian evolution, and to begin linking the dog genetic map to that of humans. These four families were designated by them OLF1, OLF2, OLF3, and OLF4 in the canine genome. The subfamilies represented by these four genes range in size from 2 to 20 genes. They are all expressed in canine olfactory epithelium but were not detectably expressed in canine lung, liver, ovary, spleen, testis, or tongue. The OLF1 and OLF2 subfamilies are tightly linked in the dog genome and also in the human genome. The smallest family is represented by the canine OLF1 gene. Using dog gene probes individually to hybridize to Southern blots of genomic DNA from 24 somatic cell hybrid lines. They showed that the human homologous OLF1 subfamily maps to human chromosome 11. The human gene with the strongest similarity to the canine OLF2 gene also mapped to chromosome 11. Both members of the human subfamily that hybridized to canine OLF3 were located on chromosome 7. It was difficult to determine to which chromosome or chromosomes the human genes that hybridized to the canine OLF4 probe mapped. This subfamily is large in mouse and hamster as well as human, so the rodent background largely obscured the human cross-hybridizing bands. It was possible, however, to discern some human-specific bands in blots corresponding to human chromosome 19. They refined the mapping of the human OLF1 homolog by hybridization to YACs that map to 11q11. In dogs, the OLF1 and OLF2 subfamilies are within 45 kb of one another (Issel-Tarver and Rine (1 996)).

Issel-Tarver and Rine (1997) demonstrated that in the human OLF1 and OLF2 homologs are likewise closely linked. By studying YACs, Issel-Tarver and Rine (1997) found that the human OLF3 homolog maps to 7q35. A chromosome 19-specific cosmid library was screened by hybridization with the canine OLF4 gene probe, and clones that hybridized strongly to the probe even at high stringency were localized to 19p13.1 and 19p13.2. These clones accounted, however, for a small fraction of the homologous human bands.

Rouquier et al. (1998) demonstrated that members of the olfactory receptor gene family are distributed on all but a few human chromosomes. Through fluorescence in situ hybridization analysis, they showed that OR sequences reside at more than 25 locations in the human genome. Their distribution was biased for terminal bands of chromosome arms. Flow-sorted chromosomes were used to isolate 87 OR sequences derived from 16 chromosomes. Their sequence relationships indicated the inter- and intrachromosomal duplications responsible for OR family expansion. Rouquier et al. (1998) determined that the human genome has accumulated a striking number of dysfunctional copies: 72% of these sequences were found to be pseudogenes. ORF-containing sequences predominate on chromosomes 7, 16, and 17.

Trask et al. (1998) characterized a subtelomeric DNA duplication that provided insight into the variability, complexity, and evolutionary history of that unusual region of the human genome, the telomere. Using a DNA segment cloned from chromosome 19, they demonstrated that the blocks of DNA sequence shared by different chromosomes can be very large and highly similar. Three chromosomes appeared to have contained the sequence before humans migrated around the world. In contrast to its multicopy distribution in humans, this subtelomeric block maps predominantly to a single locus in chimpanzee and gorilla, that site being nonorthologous to any of the locations in the human genome. Three new members of the olfactory receptor (OR) gene family were found to be duplicated within this large segment of DNA, which was found to be present at 3q, 15q, and 19p in each of 45 unrelated humans sampled from various populations. From its sequence, one of the OR genes in this duplicated block appeared to be potentially functional. The findings raised the possibility that functional diversity in the OR family is generated in part through duplications and interchromosomal rearrangements of the DNA near human telomeres.

Mombaerts (1999) reviewed the molecular biology of the odorant receptor (OR) genes in vertebrates. Buck and Axel (1991) discovered this large family of genes encoding putative odorant receptor genes. Zhao et al. (1998) provided functional proof that one OR gene encodes a receptor for odorants. The isolation of OR genes from the rat by Buck and Axel (1991) was based on three assumptions. First, ORs are likely G protein-coupled receptors, which characteristically are 7-transmembrane proteins. Second, ORs are likely members of a multigene family of considerable size, because an immense number of chemicals with vastly different structures can be detected and discriminated by the vertebrate olfactory system. Third, ORs are likely expressed selectively in olfactory sensory neurons. Ben-Arie et al. (1994) focused attention on a cluster of human OR genes on 17p, to which the first human OR gene, OR1D2, had been mapped by Schurmans et al. (1993). According to Mombaerts (1999), the sequences of more than 150 human OR clones had been reported.

The human OR genes differ markedly from their counterparts in other species by their high frequency of pseudogenes, except the testicular OR genes. Research showed that individual olfactory sensory neurons express a small subset of the OR repertoire. In rat and mouse, axons of neurons expressing the same OR converge onto defined glomeruli in the olfactory bulb.

The olfactory receptor (OR) gene family constitutes one of the largest GPCR multigene families and is distributed among many chromosomal sites in the human genome. See Rouquier et al., Hum. Mol. Genet. 7(9):1337-45 (1998); Malnic et al., Cell 96:713-23 (1999). Olfactory receptors constitute the largest family among G protein-coupled receptors, with up to 1000 members expected. See Vanderhaeghen et al., Genomics 39(3):239-46 (1997); Xie et al., Mamm. Genome 11(12):1070-78 (2000); Issel-Tarver et al., Proc. Natl. Acad. Sci. USA 93(20):10897-902 (1996). The recognition of odorants by olfactory receptors is the first stage in odor discrimination. See Krautwurst et al., Cell 95(7):917-26 (1998); Buck et al., Cell 65(1):175-87 (1991). Many ORs share some characteristic sequence motifs and have a central variable region corresponding to a putative ligand binding site. See Issel-Tarver et al., Proc. Natl. Acad. Sci. USA 93:10897-902 (1996).

G-Protein Coupled Receptor proteins (GPCRS) have been identified as a large family of G protein-coupled receptors in a number of species. These receptors share a seven transmembrane domain structure with many neurotransmitter and hormone receptors, and are likely to underlie the recognition and G-protein-mediated transduction of various signals. Examples of seven membrane spanning proteins include, serotonin receptors, dopamine receptors, histamine receptors, andrenergic receptors, cannabinoid receptors, angiotensin II receptors, chemokine receptors, opioid receptors. Human GPCR generally do not contain introns and belong to four different gene subfamilies, displaying great sequence variability. These genes are dominantly expressed in olfactory epithelium. See, e.g., Ben-Arie et al., Hum. Mol. Genet. 1994 3:229-235; and, Online Mendelian Inheritance in Man (OMIM) entry # 164342 (http://www.ncbi.nlm.nih.gov/entrez/dispomim.cgi?).

Other examples of seven membrane spanning proteins that are related to GPCRs are chemoreceptors. See Thomas et al., Gene 178(1-2): 1-5 (1996). Chemoreceptors have been identified in taste, olfactory, and male reproductive tissues. See id.; Walensky et al., J. Biol. Chem. 273(16):9378-87 (1998); Parmentier et al., Nature 355(6359):453-55 (1992); Asai et al., Biochem. Biophys. Res. Commun. 221(2):240-47 (1996).

GPCRX nucleic acids and polypeptides are useful in potential therapeutic applications implicated in various GPCR- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding a GPCRX protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The GPCRX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional GPCR-related diseases and disorders are mentioned throughout the Specification.

Further, the protein similarity information, expression pattern, and map location for GPCRX suggests that GPCRX may have important structural and/or physiological functions characteristic of the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel GPCRX substances for use in therapeutic or diagnostic methods. These antibodies may be generated according to methods known in the art, using prediction from hydrophobicity charts, as described in the “Anti-GPCRX Antibodies” section below. The disclosed GPCR1-9 proteins have multiple hydrophilic regions, each of which can be used as an immunogen. These novel proteins can also be used to develop assay systems for functional analysis.

The present invention provides novel nucleotides and polypeptides encoded thereby. Included in the invention are the novel nucleic acid sequences and their polypeptides. The sequences are collectively referred to as “GPCRX nucleic acids” or “GPCRX polynucleotides” and the corresponding encoded polypeptides are referred to as “GPCRX polypeptides” or “GPCRX proteins.” Unless indicated otherwise, “GPCRX” is meant to refer to any of the novel sequences disclosed herein. Table 1 provides a summary of the GPCRX nucleic acids and their encoded polypeptides. Also provided is a CuraGen internal Clone Identification Number for the disclosed nucleic acid and encoded polypeptides. Unless indicated otherwise, reference to a “Clone” herein refers to a discrete in silico nucleic acid sequende.

TABLE 1
Summary of Nucleic Acids and Proteins of the Invention

| GPCR | Internal Clone | Nucleic Acid | Polypeptide | Table |
|---|---|---|---|---|
| NO. | Identification No. | SEQ ID NO. | SEQ ID NO | No. |
| 1a | AC011464_A | 1 | 2 | 2 |
| 1b | AC011464_A1 | 3 | 4 | 2 |
| 2 | AC011464_B | 5 | 6 | 3 |
| 3a | GM39728201_A | 7 | 8 | 4 |
| 3b | GM39728201_A_da1 | 9 | 10 | 4 |
| 4 | AC011464_D | 11 | 12 | 5 |
| 5 | GM82965155_A | 13 | 14 | 6 |
| 6a | AC011464_F | 15 | 16 | 7 |
| 6b | dj1160k1_A_da1 | 17 | 18 | 7 |
| 7 | CG53321-03 | 19 | 20 | 8 |
| 8a | dj1160k1_A | 21 | 22 | 9 |
| 8b | CG54743-02 | 23 | 24 | 9 |
| 9 | SC80023385 | 25 | 26 | 10 |

### GPCR1

GPCR1 includes a family of two similar nucleic acids and two similar proteins disclosed below. The disclosed nucleic acids encode GPCR, OR-like proteins

### GPCR1a (AC011464_A)

GPCR1 nucleic acid is 2050 nucleotides as shown in Table 2A. As shown in Table 2A, untranslated regions 5′ to the start codon and 3′ to the stop codon are underlined, and the top codons are in bold letters.

TABLE 2A
GPCR1 nucleotide sequence.

| | |
|---|---|
| GCCACTTGCTGTTCATTAAACGTTGCTTTTTCCTCCT | (SEQ ID NO:1) |
| TCCCCAGCAGACACAACAGCTACATGGAAGCAGAAAA | |
| CCTTACAGAATTATCAAAATTTCTCCTCCTGGGACTC | |
| TCAGATGATCCTGAACTGCAGCCCGTCCTCTTTGGGC | |
| TGTTCCTGTCCATGTACCTGGTCACGGTGCTGGGGAA | |
| CCTGCTCATCATTCTGGCCGTCAGCTCTGACTCCCAC | |
| CTCCACACCCCCATGTACTTCTTCCTCTCCAACCTGT | |
| CCTTTGTTGACATCTGTTTCATCTCCACCACAGTCCC | |
| CAAGATGCTAGTGAGCATCCAGGCACGGAGCAAAGAC | |
| ATCTCCTACATGGGGTGCCTCACTCAGGTGTATTTTT | |
| TAATGATGTTTGCTGGAATGGATACTTTCCTACTGGC | |
| CGTGATGGCCTATGACCGGTTTGTGGCCATCTGCCAC | |
| CCACTGCACTACACGGTCATCATGAACCCCTGCCTCT | |
| GTGGCCTCCTGGTTCTGGCATCTTGGTTCATCATTTT | |
| CTGGTTCTCCCTGGTTCATATTCTACTGATGAAGAGG | |
| TTGACCTTCTCCACAGGCACTGAGATTCCGCATTTCT | |
| TCTGTGAACCGGCTCAGGTCCTCAAGGTGGCCTGCTC | |
| TAACACCCTCCTCAATAACATTGTCTTGTATGTGGCC | |
| ACGGCACTGCTGGGTGTGTTTCCTGTAGCTGGGATCC | |
| TCTTCTCCTACTCTCAGATTGTCTCCTCCTTAATGGG | |
| AATGTCCTCCACCAAGGGCAAGTACAAAGCCTTTTCC | |
| ACCTGTGGATCTCACCTCTGTGTGGTCTCCTTGTTCT | |
| ATGGAACAGGACTTGGGGTCTATCTGAGTTCTGCTGT | |
| GACCCATTCTTCCCAGAGCAGCTCCACCGCCTCAGTG | |
| ATGTACGCCATGGTCACCCCCATGCTGAACCCCTTCA | |
| TCTACAGCCTGAGGAACAAGGATGTGAAGGGGCCCCT | |
| GGAAAGACTCCTCAGCAGGGCCGACTCTTGTCCATGA | |
| CAAATCAGGGCCTCAGAACTAAGAGGACACACTGCGT | |
| ACCCCTAAGGCAAA | |

A disclosed encoded GPCR1a protein has 312 amino acid residues, referred to as the GPCR1 a protein. The GPCR1 a protein was analyzed for signal peptide prediction and cellular localization. SignalP results predict that GPCR1a is cleaved between position 51 and 52 of SEQ ID NO:2. Psort also predict that GPCR1 a contains a signal peptide and is likely to be localized in the plasma membrane (certainty of 0.6000). The disclosed GPCR1a polypeptide sequence is presented in Table 2B using the one-letter amino acid code.

TABLE 2B
Encoded GPCR1 protein sequence.

| | |
|---|---|
| MEAENLTELSKFLLLGLSDDPELQPVLEGLFLSMYLV | (SEQ ID NO:2) |
| TVLGNLLIILAVSSDSHLHTPMYFFLSNLSLVDICFI | |
| STTVPKMLVSIQARSKDISYMGCLTQVYFLMMFAGMD | |
| TFLLAVMAYDRFVAICHPLHYTVIMNPCLCGLLVLAS | |
| WFIIFWFSLVHILLMKRLTFSTGTEIPHFFCEPAQVL | |
| KVACSNTLLNNIVLYVATALLGVFPVAGILFSYSQIV | |
| SSLMGMSSTKGKYKAFSTCGSHLCVVSLFYGTGLGVY | |
| LSSAVTHSSQSSSTASVMYAMVTPMLNPFIYSLRNKD | |
| VKGALERLLSRADSCP | |

A BLASTX search was performed against public protein databases. The full amino acid sequence of the protein of the invention was found to have 193 of 305 amino acid residues (63%) identical to, and 242 of 305 residues (79%) positive with, the 33 amino acid residue odorant receptor protein from Rattus rattus (patp:AAR27867 Odorant receptor clone F3—Rattus rattus) (Table 2C)

TABLE 2C

BLASTX of GPRC1a against (patp:AAR27867 Odorant receptor clone F3—
Rattus rattus (SEQ ID NO:27)

Top Previous Match Next Match
Length = 333
Plus Strand HSPs:
Score = 1031 (362.9 bits), Expect 2.7e − 103, P = 2.7e − 103
Identities = 193/305 (63%), Positives = 242/305 (79%), Frame = +1

| | | | | |
|---|---|---|---|---|
| Query: | 61 | MEAENLTELSKFLLLGLSDDPELQPVLFGLFLSMYLVTVLGNLLIILAVSSDSHLHTPMY | 240 | (SEQ ID NO:2) |
| | | \|++ \| \| +\|−\|\|\|\|\| ++ +\|\|\|+++\|\|\|\|\|\|\|\|\|\|\|+\|\|+ \|\|+\|+ \|\| \|\|\|\|\|\| | 240 | |
| Sbjct: | 1 | MDSSNRTRVSEFLLLGFVENKDLQPLIYGLFLSMYLVTVIGNISIIVAIISDPCLHTPMY | 60 | (SEQ ID NO:27) |
| Query: | 241 | FFLSNLSFVDICFISTTVPKMLVSIQARSKDISYMGCLTQVYFLMMFAGMDTELLAVMAY | 420 | |
| | | \|\|\|\|\|\|\|\|\|\|\|\|\|\|\|\|\|\|\|\|\|\|\|\|+\|\| ++ \|+\| \|\|+\|\|+\|\| ++\| +\| \|\|\| +\|\|\| | 420 | |
| Sbjct: | 61 | FFLSNLSFVDICFISTTVPKMLVNIQTQNNVITYAGCITQIYFELLFVELDNFLLTIMAY | 120 | |
| Query: | 421 | DRFVAICHPLHYTVIMNPCLCGLLVLASWFIIFWFSLVHILLMKRLTFSTGTEIPHFFCE | 600 | |
| | | \|\|+\|\|\|\|\|\|+\|\|\|\|\|\|\| \|\|\| \|\|\| \|\| + +\| \|+\| \| \| \| \|\|\|\|+\|\|\| | 600 | |
| Sbjct: | 121 | DRYVAICHPMHYTVIMNYKLCGFLVLVSWIVSVLHALFQSLMMLALPFCTHLEIPHYFCE | 180 | |
| Query: | 601 | PAQVLKVACSNTLLNNIVLYVATALLGVFPVAGILFSYSQIVSSLMGMSSTKGKYKAFST | 780 | |
| | | \| \|\|+++ \|\|+ \|\|++\|+\| \|\| \|+\|\|\| +\|\| +\|\|\|\|+ +\|\| \|\|\|\|\|\|\|\| | | |
| Sbjct: | 181 | PNQVIQLTCSDAFLNDLVIYFTLVLLATVPLAGIFYSYFKIVSSICAISSVHGKYKAFST | 240 | |
| Query: | 781 | CGSHLCVVSLFYGTGLGVYLSSAVTHSSQSSSTASVMYAMVTPMLNPFIYSLRNKDVKGA | 960 | |
| | | \| \|\|\| \|\|\|\|\|\| \|\|\|\|\|\|\|\|\|\| +\|\|\|+\|+\|\|\|\|\|\|\| +\|\|\|\|+\|\|\|\|\|\|\|\|\|\|\|\|\| | | |
| Sbjct: | 241 | CASHLSVVSLFYCTGLGVYLSSAANNSSQASATASVMYTVVTPMVNPFIYSLRNKDVKSV | 300 | |
| Query: | 961 | LERLL 975 | | |
| | | \|++ \| | | |
| Sbjct: | 301 | LKKTL 305 | | |

Quantitative expression of GPCR 1a was assessed as disclosed in Example 2A.

Based on its relatedness to the GPCR superfamily proteins, the GPCR1a protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR1b (ACO11464A1)

GPCR1a nucleic acid was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide GPCR1b. The nucleotide sequence for GPCR1b (SEQ ID NO:3) is presented in Table 2D. The nucleotide sequence differs from GPCR1a by 2 nucleotide changes at positions 711 and 959.

The disclosed novel GPCR1b nucleic acid of 1050 nucleotides is shown in Table 2D. A putative untranslated region upstream from the initiation codon and downstream from the termination codon are underlined in Table 10A, and the start and stop codons are in bold letters.

TABLE 2D
GPCR1b Nucleotide Sequence

| | |
|---|---|
| GCCACTTGCTGTTCATTAAACGTTGCTTTTTCCTCCT | (SEQ ID NO:3) |
| TCCCCAGCAGACACAACAGCTACATGGAAGCAGAAAA | |
| CCTTACAGAATTATCAAAATTTCTCCTCCTGGGACTC | |
| TCAGATGATCCTGAACTGCAGCCCGTCCTCTTTGGGC | |
| TGTTCCTGTCCATGTACCTGGTCACGGTGCTGGGGAA | |
| CCTGCTCATCATTCTGGCCGTCAGCTCTGACTCCCAC | |
| CTCCACACCCCCATGTACTTCTTCCTCTCCAACCTGT | |
| CCTTTGTTGACATCTGTTTCATCTCCACCACAGTCCC | |
| CAAGATGCTAGTGAGCATCCAGGCACGGAGCAAAGAC | |
| ATCTCCTACATGGGGTGCCTCACTCAGGTGTATTTTT | |
| TAATGATGTTTGCTGGAATGGATACTTTCCTACTGGC | |
| CGTGATGGCCTATGACCGGTTTGTGGCCATCTGCCAC | |
| CCACTGCACTACACGGTCATCATGAACCCCTGCCTCT | |
| GTGGCCTCCTGGTTCTGGCATCTTGGTTCATCATTTT | |
| CTGGTTCTCCCTGGTTCATATTCTACTGATGAAGAGG | |
| TTGACCTTCTCCACAGGCACTGAGATTCCGCATTTCT | |
| TCTGTGAACCGGCTCAGGTCCTCAAGGTGGCCTGCTC | |
| TAACACCCTCCTCAATAACATTGTCTTGTATGTGGCC | |
| ACGGCACTGCTGGGTGTGTTTCCTGTAGCTGGGATCC | |
| TCTTCTCTTACTCTCAGATTGTCTCCTCCTTAATGGG | |
| AATGTCCTCCACCAAGGGCAAGTACAAAGCCTTTTCC | |
| ACCTGTGGATCTCACCTCTGTGTGGTCTCCTTGTTCT | |
| ATGGAACAGGACTTGGGGTCTATCTGAGTTCTGCTGT | |
| GACCCATTCTTCCCAGAGCAGCTCCACCGCCTCAGTG | |
| ATGTACGCCATGGTCACCCCCATGCTGAACCCCTTCA | |
| TCTACAGCCTGAGGAACAAGGATGTGAAGGGGGACCT | |
| GGAAAGACTCCTCAGCAGGGCCGACTCTTGTCCATGA | |
| CAATCAGGGCCTCAGAACTAAGAGGACACACTGCGTA | |
| CCCCTAAGGCAAA | |

The GPCR1b protein encoded by SEQ ID NO:3 has 312 amino acid residues, and is presented using the one-letter code in Table 2E (SEQ ID NO:4). The SignalP, Psort and/or Hydropathy profile for GPCR1b predict that GPCR1b has a signal peptide and is likely to be localized at the plasma membrane with a certainty of 0.6000. The SignalP predicts a cleavage site at the sequence between amino acids 51 and 52. The predicted molecular weight is 34491.4 Dal.

TABLE 2E
Encoded GPCR1b protein sequence

| | |
|---|---|
| >MEAENLTELSKFLLLGLSDDPELQPVLFGLFLSMYL | (SEQ ID NO:4) |
| VTVLGNLLIILAVSSDSHLHTPMYFFLSNLSFVDICF | |
| ISTTVPKMLVSIQARSKDISYMGCLTQVYFLMMFAGM | |
| DTFLLAVMAYDRFVAICHPLHYTVIMNPCLCGLLVLA | |
| SWFIIFWFSLVHILLMKRLTFSTGTEIPHFFCEPAQV | |
| LKVACSNTLLNNIVLYVATALLGVFPVAGILFSYSQI | |
| VSSLMGMSSTKGKYKAFSTCGSHLCVVSLFYGTGLGV | |
| YLSSAVTHSSQSSSTASVMYAMVTPMLNPFIYSLRNK | |
| DVKGDLERLLSRADSCP | |

A GPCR1b polypeptide has 207 out of 298 (69%) amino acid residues identical to and 248 out of 298 (83%) similar to the 319 amino acid residue OLFACTORY RECEPTOR—Homo sapiens (Human) (ACC:Q15622 OLFACTORY RECEPTOR).

BlastX (ptnr alignment) results include those listed in Table 2F.

TABLE 2F

Ptnr alignments of GPCR1b

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| PTNR:SPTREMBL-ACC:060412 BC62940_2 — HOMO SAPIENS (HUM . . . | +1 | 1104 | 6.1E − 111 | 1 |
| PTNR:TREMBLNEW-ACC:AAF40261 OLFACTORY RECEPTOR—GORIL . . . | +1 | 1085 | 6.3E − 109 | 1 |
| PTNR:SPTREMBL-ACC:Q15622 OLFACTORY RECEPTOR—HOMO SAP . . . | +1 | 1078 | 3.5E − 108 | 1 |
| PTNR:SPTREMBL-ACC:076100 BC85395_3—HOMO SAPIENS (HUM . . . | +1 | 1077 | 4.5 − 108 | 1 |
| PTNR:SPTREMBL-ACC:014581 OLF4—HOMO SAPIENS (HUMAN), . . . | +1 | 1074 | 9.3 − 108 | 1 |
| PTNR:SWISSPROT-ACC:Q95157 OLFACTORY RECEPTOR—LIKE PROT . . . | 1 | 1054 | 1.2 − 105 | 1 |
| PTNR:SMISSNEW-ACC:076099 OLFACTORY RECEPTOR 7C4 (OLFAC . . . | +1 | 1036 | 9.9E − 104 | 1 |

Based on its relatedness to the GPCR superfamily proteins, the GPCR1b protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR2 (AC011464_B)

A GPCR2 nucleic acid is 989 nucleotides as shown in Table 2A. As shown in Table 3A, putative untranslated regions 5′ to the start codon and 3′ to the stop codon are underlined, and the start and stop codons are in bold letters.

TABLE 3A
GPCR2 Nucleotide Sequence

| | |
|---|---|
| TTGACAAGATCAAGACTCATCAACAACATGAAAGCAG | (SEQ ID NO:5) |
| GAAACTTCTCAGACACTCCAGAATTCTTTCTCTTGGG | |
| ATTGTCAGGGGATCCGGAGCTGCAGCCCATCCTCTTC | |
| ATGCTGTTCCTGTCCATGTACCTGGCCACAATGCTGG | |
| GGAACCTGCTCATCATCCTGGCCGTCAACTCTGACTC | |
| CCACCTCCACACCCCCATGTACTTCCTCCTCTCTATC | |
| GTGTCCTTGGTCGACATCTGTTTCACCTCCACCACGA | |
| TGCCCAAGATGCTGGTGAACATCCAGGCACAGGCTCA | |
| ATCCATCAATTACACAGGCTGCCTCACCCAAATCTGC | |
| TTTGTCCTGGTTTTTGTTGGATTGGAAAATGGAATTC | |
| TGGTCATGATGGCCTATGATCGATTTGTGGCCATCTG | |
| TCACCCACTGAGGTACAATGTCATCATGAACCCCAAA | |
| CTCTGTGGGCTGCTGCTTCTGCTGTCCTTCATCGTTA | |
| GTGTCCTGGATGCTCTGCTGCACACGTTGATGGTGCT | |
| ACAGCTGACCTTCTGCATAGACCTGGAAATTCCCCAC | |
| TTTTTCTGTGAACTAGCTCATATTCTCAAGCTCGCCT | |
| GTTCTGATGTCCTCATCAATAACATCCTGGTGTATTT | |
| GGTGACGAGCCTGTTAGGTGTTGTTCCTCTCTCTGGG | |
| ATCATTTTCTCTTACACACGAATTGTCTCCTCTGTCA | |
| TGAAAATTCCATCAGCTGGTGGAAAGTATAAAGCTTT | |
| TTCCATCTGCGGGTCACATTTAATCGTTGTTTCCTTG | |
| TTTTATGGAACAGGGTTTGGGGTGTACCTTAGTTCTG | |
| GGGCTACCCACTCCTCCAGGAAGGGTGCAATAGCATC | |
| AGTGATGTATACCGTGGTCACCCCCATGCTGAACCCA | |
| CTCATTTACAGCCTGAGAAACAAGGACATGTTGAAGG | |
| CTTTGAGGAAACTAATATCTAGGATACCATCTTTCCA | |
| TTGATGTCTCAGCTTCTTGGGCTTACA | |

The disclosed GPCR2 polypeptide (SEQ ID NO:6) encoded by SEQ ID NO:5 is 312 amino acid residues and is presented using the one-letter code in Table 3B. The GPCR2 protein was analyzed for signal peptide prediction and cellular localization. SignalP results predict that GPCR2 is cleaved between position 51 and 52 of SEQ ID NO:6. Psort and Hydropathy profiles also predict that GPCR2 contains a signal peptide and is likely to be localized at the plasma membrane (certainty of 0.6000). The predicted molecular weight is 34438.9 Dal.

TABLE 3B
Encoded GPCR2 protein sequence.

| | |
|---|---|
| MKAGNFSDTPEFFLLGLSGDPELQPILFMLFLSMYLA | (SEQ ID NO:6) |
| TMLGNLLIILAVNSDSHLHTPMYFLLSILSLVDICFT | |
| STTMPKMLVNIQAQAQSINYTGCLTQICFVLVFVGLE | |
| NGILVMMAYDRFVAICHPLRYNVIMNPKLCGLLLLLS | |
| FIVSVLDALLHTLMVLQLTFCIDLEIPHFFCELAHIL | |
| KLACSDVLINNILVYLVTSLLGVVPLSGIIFSYTRIV | |
| SSVMKISAGGKYKAFSICGSHLIVVSLFYGTGFGVYL | |
| SSGATHSSRKGAIASVMYTVVTPMLNPLIYSLRNKDM | |
| LKALRKLISRIPSFH | |

A BLASTX search was performed against public protein databases. The GPCR2 polypeptide has 184 out of 305 (60%) amino acid residues identical to and 228 out of 305 similar to the 333 amino acid residue Rattus rattus odorant receptor clone F3 (patp:AAR27867) (Table 3C).

TABLE 3C

BLASTX of GPCR2 against (patp:AAR27867 Odorant receptor clone F3—
Rattus rattus (SEQ ID NO:28)

Top Previous Match Next Match
Length = 333
Plus Strand HSPs:
Score = 947 (333.4 bits), Expect = 2.2e − 94, P = 2.2e − 94
Identities = 184/305 (60%), Positives = 228/305 (74%), Frame = +1

| | | | | |
|---|---|---|---|---|
| Query: | 28 | MKAGNFSDTPEFFLLGLSGDPELQPILFMLFLSMYLATMLGNLLIILAVNSDSHLHTPMY | 207 | (SEQ ID NO:6) |
| | | \| − \| + \|\| \|\|\| + +\|\|\|+++ \|\|\|\|\|\|\| \|++\|\|+ \|\|+\|\|+ \|\| \|\|\|\|\|\| | | |
| Sbjct: | 1 | MDSSNRTRVSEFLLLGFVENKDLQPLIYGLFLSMYLVTVIGNISIIVAIISDPCLHTPMY | 60 | (SEQ ID NQ:28) |
| Query: | 208 | FLLSILSLVDICFTSTTMPKMLVNIQAQAQSINYTGCLTQICFVLVENGILVMMAY | 387 | |
| | | \| \|\| \|\| \|\|\|\|\| \|\|\|+\|\|\|\|\|\|\|\| \| \| \| \|\|+\|\|\| \| \|+\|\| \|+\| +\| +\|\|\| | | |
| Sbjct: | 61 | FFLSNLSFVDICFISTTVPKMLVNIQTQNNVITYAGCITQIYFFLLFVELDNFLLTIMAY | 120 | |
| Query: | 388 | DRFVAICHPLRYNVIMNPKLCGLLLLLSFIVSVLDALLHTLMVLQLTFCIDLEIPHFFCE | 567 | |
| | | \|\|+\|\|\|\|\|+ \| \|\|″\| \|+\|+\|+\|\|\|\|\| \|\| +\|\|+\| \| \|\| \|\|\|\|\|+\|\|\| | | |
| Sbjct: | 121 | DRYVAICHPMHYTVIMNYKLCGFLVLVSWIVSVLHALFQSLMMLALPFCTHLEIPHYFCE | 180 | |
| Query: | 568 | LAHILKLACSDVLINNILVYLVTSLLGVVPLSGIIFSYTRIVSSVMKIPSAGGKYKAFSI | 747 | |
| | | +++\| \|\|\| +\|++++\| \|\| \|\|\|+\|\| +\|\| +\|\|\|\|+ \| \| \|\|\|\|\|\|\| | | |
| Sbjct: | 181 | PNQVIQLTCSDAFLNDLVIYFTLVLLATVPLAGIFYSYFKIVSSICAISSVHGKYKAFST | 240 | |
| Query: | 748 | CGSHLIVVSLFYGTGFGVYLSSGATHSSRKGAIASVMYTVVTPMLNPLIYSLRNKDMLKA | 927 | |
| | | \| \|\|\| \|\|\|\|\|\| \|\| \|\|\|\|\|\| \| +\|\|+ \| \|\|\|\|\|\|\|\|\|\|\|+\|\| \|\|\|\|\|\|\|\|+ | | |
| Sbjct: | 241 | CASHLSVVSLFYCTGLGVYLSSAANNSSQASATASVMYTVVTPMVNPFIYSLRNKDVKSV | 300 | |
| Query: | 928 | LRKLI 942 | | |
| | | \|+\| + | | |
| Sbjct: | 301 | LKKTL 305 | | |

The GPCR2 nucleic acid was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy. There are no amino acid and nucleotide differences between GPCR2 and the resulting amplicon.

Quantitative expression of GPCR 2 was assessed as disclosed in Example 3B.

Based on its relatedness to the GPCR superfamily proteins, the GPCR2 protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR3

GPCR3 includes a family of two similar nucleic acids and two similar proteins disclosed below. The disclosed nucleic acids encode GPCR, OR-like proteins.

### GPCR3a (GM39728201_A)

The disclosed GPCR3a nucleic acid is 1061 nucleotides as shown in Table 4A. As shown in Table 3A, putative untranslated regions 5′ to the start codon and 3′ to the stop codon are underlined, and the start and stop codons are in bold letters.

TABLE 4A
GPCR3a Nucleotide Sequence

| | |
|---|---|
| TCGAGTGATGCCGATGCAGCTGCTGCTTACAGATTTT | (SEQ ID NO:7) |
| ATTATCTTTTCCATCAGATTCATCATCAACAGCATGG | |
| AAGCGAGAAACCAAACAGCTATTTCAAAATTCCTTCT | |
| CCTGGGACTGATAGAGGATCCGGAACTGCAGCCCGTC | |
| CTTTTCAGCCTGTTCCTGTCCATGTACTTGGTCACCA | |
| TCCTGGGGAACCTGCTCATCCTCTTGGCTGTCATCTC | |
| TGACTCTCACCTCCACACCCCCATGTACTTCTTCCTC | |
| TCCAATCTCTCCTTTTTGGACATTTGTTTAAGCACAA | |
| CCACGATCCCAAAGATGCTGGTGAACATCCAAGCTCA | |
| GAATCGGAGCATCACGTACTCAGGCTGCCTCACCCAG | |
| ATCTGCTTTGTCTTGTTTTTTGCTGGCTTGGAAAATT | |
| GTCTCCTTGCAGCAATGGCCTATGACCGCTATGTGGC | |
| CATTTGTCACCCCCTTAGATACACAGTCATCATGAAC | |
| CCCCGCCTCTGTGGCCTGCTGATTCTTCTCTCTCTGT | |
| TGACTAGTGTTGTGAATGCCCTTCTTCTCAGCCTGAT | |
| GGTGTTGAGGCTGTCCTTCTGCACAGACCTGGAAATC | |
| CGCCTCTTCTTCTGTGAACTGGCTCAGGTCATCCAAC | |
| TCACCTGTTCAGACACCCTCATCAATAACATCCTGAT | |
| ATATTTTGCAGCTTGCATATTTGGTGGTGTTCCTCTG | |
| TCTGGAATCATTTTGTCTTACACTCAGATCACCTCCT | |
| GTGTTTTGAGAATGCCATCAGCAAGTGGAAAGCACAA | |
| AGCAGTTTCCACCTGTGGGTCTCACCTCTCCATTGTT | |
| CTCTTGTTCTATGGGGCAGGTTTGGGGGTGTACATTA | |
| GTTCTGTGGTTACTGACTCACCTAGGAAGACTGCAGT | |
| GGCTTCAGTGATGTATTCTGTGTTCCCTCAAATGGTG | |
| AACCCCTTTATCTATAGTCTGAGGAATAAGGACATGA | |
| AAGGAACCTTGAGGAAGTTCATAGGGAGGATACCTTC | |
| TCTTCTGTGGTGTGCCATTTGCTTTGGATTCAGGTTT | |
| CTAGAGTAAGTCAAAGTGACAGGAT | |

The disclosed GPCR3a polypeptide (SEQ ID NO:8) encoded by SEQ ID NO:7 is 345 amino acid residues and is presented using the one-letter code in Table 4B. SignalP results predict that GPCR3a is cleaved between position 51 and 52 of SEQ ID NO:8. Psort and Hydropathy profiles also predict that GPCR3a contains a signal peptide and is likely to be localized in the plasma membrane (certainty of 0.6000). The predicted molecular height is 38425.5 Dal.

TABLE 4B
Encoded GPCR3a protein sequence (SEQ ID NO:8).

MPMQLLLTDFIIFSIRFIINSMEARNQTAISKFLLLGLIEDPELQPVLFSLFLSMYLVTILGNLLILLAVISDSH
LHTPMYFFLSNLSFLDICLSTTTIPKMLVNIQAQNRSITYSGCLTQICFVLFFAGLENCLLAAMAYDRYVAICHP
LRYTVIMNPRLCGLLILLSLLTSVVNALLLSLMVLRLSFCTDLEIPLFFCELAQVIQLTCSDTLINNILIYFAAC
IFGGVPLSGIILSYTQITSCVLRMPSASGKHKAVSTCGSHLSIVLLFYGAGLGVYISSVVTDSPRKTAVASVMYS
VFPQMVNPFIYSLRNKDMKGTLRKFIGRIPSLLWCAICFGFRFLE

A GPCR3a polypeptide has 185 out of 303 (61%) amino acid residues identical to and 235 out of 303 similar to the 333 amino acid residue Rattus rattus odorant receptor clone F3 (patp:AAR27867) (Table 4C)

TABLE 4C
BLASTX of GPCR3a against patp:AAR27867 Odorant receptor clone F3 -
Rattus rattus (SEQ ID NO:29)

Top Previous Match Next Match
Length = 333
Plus Strand HSPs:
Score = 981 (345.3 bits), Expect = 5.4e−98, P = 5.4e−98
Identities 185/303 (61%), Positives = 235/303 (77%), Frame = +2

| | | | | |
|---|---|---|---|---|
| Query: | 71 | MEARNQTAISKFLLLCLIEDPELQPVLFSLFLSMYLVTILGNLLILLAVISDSHLHTPMY | 250 | (SEQ ID NO:8) |
| | | \|++ \|+\| +\|+\|\|\|\|\| +\|+ +\|\|\|+++ \|\|\|\|\|\|\|\|\|++\|\|+ \|++\|+\|\|\| \|\|\|\|\|\| | | |
| Sbjct: | 1 | MDSSNRTRVSEFLLLGFVENKDLQPLIYGLFLSMYLVTVIGNISIIVAIISDPCLHTPMY | 60 | (SEQ ID NO:29) |
| Query: | 251 | FFLSNLSFLDICLSTTTIPKMLVNIQAQNRSITYSGCLTQICFVLFFAGLENCLLAAMAY | 430 | |
| | | \|\|\|\|\|\|\|\|+\|\|\| +\|\|+\|\|\|\|\|\|\|\| \|\| \|\|\|+\|\|+\|\|\| \| \| \| \|+\| \|\| \|\|\| | | |
| Sbjct: | 61 | FFLSNLSFVDICFISTTVPKMLVNTQTQNNVITYAGCITQIYFFLLFVELDNFLLTIMAY | 120 | |
| Query: | 431 | DRYVAICHPLRYTVIMNPRLCGLLILLSLLTSVVNALLLSLMVLRLSFCTDLEIPLFFCE | 610 | |
| | | \|\|\|\|\|\|\|\|\|+ \|\|\|\|\|\| +\|\|\| \|+\|+\| + \|\|++\|\| \|\|\|+\| \| \|\|\| \|\|\|\| +\|\|\| | | |
| Sbjct: | 121 | DRYVAICHPMHYTVIMNYKLCGFLVLVSWIVSVLHALFQSLMMLALPFCTHLEIPHYFCE | 180 | |
| Query: | 611 | LAQVIQLTCSDTLINNILIYFAACIFGGVPLSGIILSYTQITSCVLRMPSASGKHKAVST | 790 | |
| | | \|\|\|\|\|\|\|\|\| +\|+++\|\|\| + \|\|\|+\|\| \|\| +\| \| + + \| \|\|+\|\| \|\| | | |
| Sbjct: | 181 | PNQVIQLTCSDAFLNDLVIYFTLVLLATVPLAGIFYSYFKIVSSICAISSVHGKYKAFST | 240 | |
| Query: | 791 | CGSHLSIVLLFYGAGLGVYISSVVTDSPRKTAVASVMYSVFPQMVNPFIYSLRNKDMKGT | 970 | |
| | | \| \|\|\|\|+\| \|\|\| \|\|\|\|\|+\|\| +\| + +\| \|\|\|\|\|+\| \|\|\|\|\|\|\|\|\|\|\|\|\|+\| | | |
| Sbjct: | 241 | CASHLSVVSLFYCTGLGVYLSSAANNSSQASATASVMYTVVTPMVNPFIYSLRNKDVKSV | 300 | |
| Query: | 971 | LRK | 979 | |
| | | \|+\| | | |
| Sbjct: | 301 | LKK | 303 | |

Single Nucleotide Polymorphisms (SNPs) were identified in a GPCR3a nucleic acid. The positions of the SNPs are listed in Table 4D.

TABLE 4D
cSNPs

| Base Position | | | Amino Acid |
|---|---|---|---|
| of cSNP | Wild Type | Variant | Change |
| 140 | C | A | Val-Phe |
| 192 | C | T | Met-Ile |

Quantitative expression of GPCR3a was assessed as disclosed in Example 3C.

Based on its relatedness to the GPCR superfamily proteins, the GPCR3a protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR3b (GM39728201_A_dal)

GPCR3a nucleic acid was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide GPCR3b. The nucleotide sequence for GPCR3b (SEQ ID NO:9) is presented in Table 4E. The nucleotide sequence differs from GPCR3a by 4 nucleotide changes at positions 144, 793, 846 and 899. The encoded GPCR3b protein is 21 amino acids shorter than GPCR3a on the N-terminal end and has differences as amino acids 48, 263 and 282.

TABLE 4E
GPCR3b Nucleotide Sequence (SEQ ID NO:9)

ACAGCATGGAAGCGAGAAACCAAACAGCTATTTCAAAATTCCTTCTCCTGGGACTGATAGAGGATCCGGA
ACTGCAGCCCGTCCTTTTCAGCCTGTTCCTGTCCATGTACTTGGTCACCATCCTGGGGAACCTGCTCATC
CTCATGGCTGTCATCTCTGACTCTCACCTCCACACCCCCATGTACTTCTTCCTCTCCAATCTCTCCTTTT
TGGACATTTGTTTAAGCACAACCACGATCCCAAAGATGCTGGTGAACATCCAAGCTCAGAATCGGAGCAT
CACGTACTCAGGCTGCCTCACCCAGATCTGCTTTGTCTTGTTTTTTGCTGGCTTGGAAAATTGTCTCCTT
GCAGCAATGGCCTATGACCGCTATGTGGCCATTTGTCACCCCCTTAGATACACAGTCATCATGAACCCCC
GCCTCTGTGGCCTGCTGATTCTTCTCTCTCTGTTGACTAGTGTTGTGAATGCCCTTCTTCTCAGCCTGAT
GGTGTTGAGGCTGTCCTTCTGCACAGACCTGGAAATCCCGCTCTTCTTCTGTGAACTGGCTCAGGTCATC
CAACTCACCTGTTCAGACACCCTCATCAATAACATCCTGATATATTTTGCAGCTTGCATATTTGGTGGTG
TTCCTCTGTCTGGAATCATTTTGTCTTACACTCAGATCACCTCCTGTGTTTTGAGAATGCCATCACCAAG
TGGAAAGCACAAAGCAGTTTCCACCTGTGGGTCTCACCTCTCCATTGTTCTCTTGTTCTATGGGGCAGCT
TTGGGGGTGTACATTAGTTCTGCGGTTACTGACTCACCTAGGAAGACTGCAGTGGCTTCAGTGATGTATT
CTGTGGTCCCTCAAATGGTGAACCCCTTTATCTATAGTCTGAGGAATAAGGACATGAAGGGAACCTTGAG
GAAGTTCATAGGGAGGATACCTTCTCTTCTGTGGTGTGCCATTTGCTTTGGATTCAGGTTTCTAGAGTAA
GTCA

The encoded GPCR3b protein is presented in Table 4F. The disclosed protein is 325 amino acids long and is denoted SEQ ID NO:10. The predicted molecular weight is 335872.4 Dal.

TABLE 4F
Encoded GPCR3b protein sequence (SEQ ID NO:10).

MEARNQTAISKFLLLGLIEDPELQPVLFSLFLSMYLVTILGNLLILMAVISDSHLHTPMYFFLSNLS
FLDICLSTTTIPKMLVNIQAQNRSITYSGCLTQICFVLFFAGLENCLLAAMAYDRYVAICHPLRYTV
IMNPRLCGLLILLSLLTSVVNALLLSLMVLRLSFCTDLEIPLFFCELAQVIQLTCSDTLINNILIYF
AACIFGGVPLSGIILSYTQITSCVLRMPSASGKHKAVSTCGSHLSIVLLFYGAGLGVYISSAVTDSP
RKTAVASVMYSVVPQMVNPFIYSLRNKDMKGTLRKFIGRIPSLLWCAICFGFRFLE

A GPCR3b polypeptide has 202 of 308 (65%) amino acid residues identical to and 244 of 308 (79%) similar to the 309 amino acid residue SPTREMPL-ACC: 014581 OLF4—Homo sapiens (Human) (Table 4G).

TABLE 4G
BLASTX of GPPCR3b against SPTREMPL-ACC: 014581 OLF4 -
Homo sapiens (Human) (SEQ ID NO:30)

>ptnr.SPTREMBL-ACC:014581 OLF4 - Homo sapiens (Human), 309 aa.
Top Previous Match Next Match
Length = 309
Plus Strand HSPs:
Score = 1043 (367.2 bits), Expect = 1.8e−104, P = 1.9e−104
Identities = 202/309 (65%), Positives = 244/308 (79%), Frame = +3

| | | | | |
|---|---|---|---|---|
| Query: | 6 | MEARNQTAISKFLLLGLIEDPELQPVLFSLFLSMYLVTILGNLLILMAVISDSHLHTPMY | 185 | (SEQ ID NO:10) |
| | | \|\| \| \| \|\|+\|+\|\|\|\| \|+\|\|\|\|\| \|\| \|\|\|\|\|\|\|\|\|+\|\|\|\|\|\|++\| \|\|\|\|\|\|\|\|\|\|\| | | |
| Sbjct: | 1 | MEPENDTGISEFVLLGLSEEPELQPFLFGLFLSMYLVTVLGNLLIILATISDSHLHTPMY | 60 | (SEQ ID NO:30) |
| Query: | 186 | FFLSNLSFLDICLSTTTIPKMLVNIQAQNRSITYSGCLTQICFVLFFAGLENCLLAAMAY | 365 | |
| | | \|\|\|\|\|\|\|\| \|\|\| +\|\|\|\|\|\|\|+\|\|\| \|+\| \|\|\|+\|\|+\|\|+\|\| + \| \|\|++ \|\|\| \|\|\| | | |
| Sbjct: | 61 | FFLSNLSFADICFISTTIPKMLINIQTQSRVITYAGCITQMCFFVLFGGLDSLLLAVMAY | 120 | |
| Query: | 366 | DRYVAICHPLRYTVIMNPRLCGLLILLSLLTSVVNALLLSLMVLRLSFCTDLEIPLFFCE | 545 | |
| | | \|\|+\|\|\|\|\|\|\| \|\|\|\|\|\|\|\|\|\|\|\|\|+\| \| + + +\|+\| \|\|\|\|\| \|\|\|\|\|\|\|\|\|\| \|\|\|\| | | |
| Sbjct: | 121 | DRFVAICHPLHYTVIMNPRLCGLLVLASWMIAALNSLSQSLMVLWLSFCTDLEIPHFFCE | 180 | |
| Query: | 546 | LAQVIQLTCSDTLINNILIYFAACIFGGVPLSGIILSYTQITSCVLRMPSASGKHKAVST | 725 | |
| | | \| \|\|\| \| \|\|\|\| +\|++ +\|\|\|\| + \| \|\| \|\|+ \|\|++\| \| + + \|\| \|\|+\|\| \|\| | | |
| Sbjct: | 181 | LNQVIHLACSDTFLNDMGMYFAAGLLAGGPLVGILCSYSKIVSSIRAISSAQGKYKAFST | 240 | |
| Query: | 726 | CGSHLSIVLLFYGAGLGVYISSAVTDSPRKTAVASVMYSVVPQMVNPFIYSLRNKDMKGT | 905 | |
| | | \| \|\|\|\|+\| \|\| \|\|\|\|\|++\|\| \| + +\| \|\|\|\|\|+\| \|+\|\|\|\|\|\|\|\|\|\|\|+\| | | |
| Sbjct: | 241 | CASHLSVVSLFCCTGLGVYLTSAATHNSHTSATASVMYTVATPMLNPFIYSLRNKDIKRA | 300 | |
| Query: | 906 | LR-KFIGR | 926 | |
| | | \|+ \| \|+ | | |
| Sbjct: | 301 | LKMSFRGK | 308 | |

PSORT analysis predicts the protein of the invention to be localized to the endoplasmic reticulum with a certainty of 0.6850. Using the SignalP analysis, it is predicted that the protein of the invention may have a signal peptide with most likely cleavage site between positions 51 and 52 (SEQ ID NO:10)

Based on its relatedness to the GPCR superfamily proteins, the GPCR3b protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR4 (ACO11464_D)

The disclosed GPCR4 nucleic acid is 1050 nucleotides as shown in Table 5A. As shown in Table 5A, putative untranslated regions 5′ to the start codon and 3′ to the stop codon are underlined, and the start and stop codons are in bold letters.

TABLE 5A
GPCR4 Nucleotide Sequence (SEQ ID NO:11)

TGCTCCCGCAGTCTAGAAAACACATTTGATTGTCTAATTATCCCATTTTTTGATCATCAAAAACATGGGA
CCCAGAAACCAAACAGCTGTTTCAGAATTTCTTCTCATGAAAGTGACAGAGGACCCAGAACTGAAGTTAA
TCCCTTTCAGCCTGTTCCTGTCCATGTACCTGGTCACCATCCTGGGGAACCTGCTCATTCTCCTGGCTGT
CATCTCTGACTCCCACCTCCACACCCCCATGTACTTCCTTCTCTTTAATCTCTCCTTTACTGACATCTGT
TTAACCACAACCACAGTCCCAAAGATCCTAGTGAACATCCAAGCTCAGAATCAGAGTATCACTTACACAG
GCTGCCTCACCCAGATCTGTCTTGTCTTGGTTTTTGCTGGCTTGGAAAGTTGCTTTCTTGCAGTCATGGC
CTACGACCGCTATGTGGCCATTTGCCACCCACTGAGGTACACAGTCCTCATGAATGTCCATTTCTGGGGC
TTCCTGATTCTTCTCTCCATGTTCATGAGCACTATGGATGCCCTGGTTCAGAGTCTGATGGTATTGCAGC
TGTCCTTCTGCAAAAACGTTGAAATCCCTTTGTTCTTCTGTGAAGTCGTTCAGGTCATCAAGCTCGCCTG
TTCTGACACCCTCATCAACAACATCCTCATATATTTTGCAAGTAGTGTATTTGGTGCAATTCCTCTCTCT
GGAATAATTTTCTCTTATTCTCAAATAGTCACCTCTGTTCTGAGAATGCCATCAGCAAGAGGAAAGTATA
AAGCGTTTTCCACCTGTGGCTGTCACCTCTCTGTTTTTTCCTTGTTCTATGGGACAGCTTTTGGGGTGTA
CATTAGTTCTGCTGTTGCTGAGTCTTCCCGAATTACTGCTGTGGCTTCAGTGATGTACACTGTGGTCCCT
CAAATGATGAACCCCTTCATCTACAGCCTGAGAAATAAGGAGATGAAGAAAGCTTTGAGGAAACTTATTG
GTAGGCTGTTTCCTTTTTAGCGATCTTTGTCCTCTGCTTTGAATTGGAGCTTCTAAAATAAATCATCATA

The disclosed GPCR4 polypeptide (SEQ ID NO:12) encoded by SEQ ID NO:11 is 311 amino acid residues and is presented using the one-letter code in Table 5B. SignalP results predict that GPCR4 is cleaved between position 53 and 54 of SEQ ID NO:12. Psort and Hydropathy profiles also predict that GPCR4 contains a signal peptide and is likely to be localized in the plasma membrane (certainty of 0.6000). The predicted molecular height is 34802.2 Dal.

TABLE 5B
Encoded GPCR4 protein sequence (SEQ ID NO:12).

MGPRNQTAVSEFLLMKVTEDPELKLIPFSLFLSMYLVTILGNLLILLAVISDSHLHTPMYFLLFNLSFTDICLTT
TTVPKILVNIQAQNQSITYTGCLTQICLVLVFAGLESCFLAVMAYDRYVAICHPLRYTVLMNVHFWGLLILLSMF
MSTMDALVQSLMVLQLSFCKNVEIPLFFCEVVQVIKLACSDTLINNILIYFASSVFGAIPLSGIIFSYSQIVTSV
LRMPSARGKYKAFSTCGCHLSVFSLFYGTAFGVYISSAVAESSRITAVASVMYTVVPQMMNPFIYSLRNKEMKKA
LRKLIGRLFPF

A GPCR4 polypeptide has 156 out of 237 (65%) amino acid residues identical to and 197 out of 237 (80%) similar to the 237 amino acid residue marmot olfactory receptor protein AMOR7 —Marmota marmota (patp: AA54332) (Table 5C).

TABLE 5C
BLASTX of GPCR4 against marmot olfactory receptor protein AMOR7-
Marmota marmota (patp: AA54332) (SEQ ID NO:31)

Top Previous Match Next Match
Length = 237
Plus Strand HSPs:
Score = 822 (289.4 bits), Expect = 3.8e−81, P = 3.8e−81
Identities = 156/237 (65%), Positives = 191/237 (80%), Frame = +2

| | | | | |
|---|---|---|---|---|
| Query: | 236 | PMYFLLFNLSFTDICLTTTTVPKILVNIQAQNQSITYTGCLTQICLVLVFAGLESCFLAV | 415 | (SEQ ID NO:11) |
| | | \| \| \| \|\|\| \|\| ++\|\|\|+\|+++\|\|\|\| + ++\|+\| \|\|\|\|\|+\| \|\|+\|\|\| \|+ \|\| | | |
| Sbjct: | 1 | PRYLFLGNLSLADIGISTTTIPQMVVNIQRKRKTISYAGCLTQVCFVLTFAGSENFLLAA | 60 | (SEQ ID NO:31) |
| Query: | 416 | MAYDRYVAICHPLRYTVLMNVHFWGLLILLSMFMSTMDALVQSLMVLQLSFCKNVEIPLF | 595 | |
| | | \|\|\|\|\|\| \|\|\|\|\|\|\|\|\| +\|\| \| \|\|+++\|+ +\|\|+\|\|\|+ \|\|\|+\|+\|\|\|\| ++\|\|\| \| | | |
| Sbjct: | 61 | MAYDRYAAICHPLRYTAIMNPHLCVLLVMISLSISTVDALLHSLMLLRLSFCTDLEIPHF | 120 | |
| Query: | 596 | FCEVVQVIKLACSDTLINNILIYFASSVFGAIPLSGIIFSYSQIVTSVLRMPSARGKYKA | 775 | |
| | | \|\|\|+ \|\|\| \|\|\|\|\|\|\|\|\|\|+\|\|\| + +\| +\|\|\|\|\|\|\|\|\| \|\|+\|\|\|\|\|\|\| \| \|\|\| | | |
| Sbjct: | 121 | FCELDQVITLACSDTLINNLLIYVTAGIFAGVPLSGIIFSYLHIVSSVLRMPSPGGVYKA | 180 | |
| Query: | 776 | FSTCGCHLSVFSLFYGTAFGVYISSAVAESSRITAVASVMYTVVPQMMNPFIYSLRN | 946 | |
| | | \|\|\|\|\| \|\|\|\| \|\|\|\|\| \|\|\|\|\|\|\|\|\| +\| \| \|\|\|\|\|\|\|+\|\|\|\|\|+\|\| \|\|+\|\|\| | | |
| Sbjct: | 181 | FSTCGSHLSVVCLFYGTIFGVYISSAVTDSQRKGAVASVMYSVVPQMLNPIIYTLRN | 237 | |
| Query: | | | | |
| Sbjct: | | | | |

Quantitative expression of GPCR4 was assessed as disclosed in Example 3D.

Based on its relatedness to the GPCR superfamily proteins, the GPCR4 protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR5 (GM82965155_A)

The disclosed novel GPCR5 nucleic acid of 980 nucleotides is shown in Table 6A. A putative untranslated region upstream from the initiation codon and downstream from the termination codon is underlined in Table 6A, and the start and stop codons are in bold letters.

TABLE 6A
GPCR5 Nucleotide Sequence (SEQ ID NO:13)

CTTCTTTTGTAGATACATCAGCTACATGGAAGCAGGAAACCAAACAGGATTTTTAGAGTTTATCCTTCTC
GGACTCTCTGAGGATCCAGAACTACAGCCGTTCATATTTGGGCTGTTCCTGTCCATGTACCTGGTGACGG
TGCTGGGAAACCTGCTCATCATCCTGGCCATCAGCTCTGACTCCCACCTCCACACCCCCATGTACTTCTT
CCTCTCCAACCTGTCCTGGGTTGACATCTGTTTCAGCACTTGCATCGTCCCCAAGATGCTGGTGAACATC
CAGACCGAGAACAAAGCCATCTCCTACATGGACTGCCTCACACAGGTCTATTTCTCCATGTTTTTTCCTA
TTCTGGACACGCTACTCCTGACCGTGATGGCCTATGACCGGTTTGTGGCTGTCTGCCACCCTCTGCACTA
TATGATCATCATGAACCCCCACCTCTGTGGCCTCCTGGTTTTTGTCACCTGGCTCATTGGTGTCATGACA
TCCCTCCTCCATATTTCTCTGATGATGCATCTAATCTTCTGTAAAGATTTTGAAATTCCACATTTTTTCT
GCGAACTGACGTACATCCTCCAGCTGGCCTGCTCTGATACCTTCCTGAACAGCACGTTGATATACTTTAT
GACGGGTGTGCTGGGCGTTTTTCCCCTCCTTGGGATCATTTTCTCTTATTCACGAATTGCTTCATCCATA
AGGAAGATGTCCTCATCTGGGGGAAAACAAAAAGCACTTTCCACCTGTGGGTCTCACCTCTCCGTCGTTT
CTTTATTTTATGGGACAGGCATTGGGGTCCACTTCACTTCTGCGGTGACTCACTCTTCCCAGAAAATCTC
CGTGGCCTCGGTGATGTACACTGTGGTCACCCCCATGTTGAACCCCTTCATCTACAGCCTGAGGAACAAG
GATGTGAAGGGAGCCCTGGGGAGTCTCCTCAGCAGGGCAGCCTCTTGTTTGTGATGGATCCCTTGGCCCC

The GPCR5 protein (SEQ ID NO:14) encoded by SEQ ID NO:13 has 312 amino acid residues and is presented using the one-letter code in Table 6B. The Psort profile for GPCR5 predicts that this sequence has a signal peptide and is likely to be localized in the plasma membrane with a certainty of 0.6000. The most likely cleavage site for a peptide is between amino acids 48 and 49, based on the SignalP result. The predicted molecular weight is 34746.8 Dal.

TABLE 6B
Encoded GPCR5 protein sequence (SEQ ID NO:14)

MEAGNQTGFLEFILLGLSEDPELQPFIFGLFLSMYLVTVLGNLLIILAISSDSHLHTPMYFFLSN
LSWVDICFSTCIVPKMLVNIQTENKAISYMDCLTQVYFSMFFPILDTLLLTVMAYDRFVAVCHPL
HYMIIMNPHLCGLLVFVTWLIGVMTSLLHISLMMHLIFCKDFEIPHFFCELTYILQLACSDTFLN
STLIYFMTGVLGVFPLLGIIFSYSRIASSIRKMSSSGGKQKALSTCGSHLSVVSLFYGTGIGVHF
TSAVTHSSQKISVASVMYTVVTPMLNPFIYSLRNKDVKGALGSLLSRAASC

A GPCR5 polypeptide has been found to have 185 out of 305 (60%) amino acid residues identical to, and 228 out of 305 (74%) similar to the 333 amino acid residue Odorant receptor clone F3—Rattus rattus (patp: AAR27867). (Table 6C)

TABLE 6C
BLASTX of GPCR5 against Odorant receptor clone
F3 - Rattus rattus (patp:AAR27867) (SEQ ID NO:32)

```
Top Previous Match Next Match
                        Length = 333
    Plus Strand HSPs:
  Score = 972 (342.2 bits), Expect = 4.9e−97, P = 4.9e−97
  Identities = 185/305 (60%), Positives = 228/305 (74%), Frame = +30 2
```

| | | |
|---|---|---|
| Query: | 26 MEAGNQTGFLEFILLGLSEDPELQPFIFGLFLSMYLVTVLGNLLIILAISSDSHLHTPMY 205 | (SEQ ID NO:14) |
| | \|++ \|+\| \|\|+\|\|\| \|+ +\|\|\| \|+\|\|\|\|\|\|\|\|\|\|\|+\|\|+ \|\|+\|\| \|\| \|\|\|\|\|\| | |
| Sbjct: | 1 MDSSNRTRVSEFLLLGFVENKDLQPLIYGLFLSMYLVTVIGNISIIVAIISDPCLHTPMY 60 | (SEQ ID NO:32 |
| Query: | 206 FFLSNLSWVDICFSTCIVPKMLVNIQTENKAISYMDCLTQVYFSMFFPILDTLLLTVMAY 385 | |
| | \|\|\|\|\|\|\|+\|\|\|\|\| + \|\|\|\|\|\|\|\|\|\|+\| \|+\| \|+\|\|+\|\| + \| \|\| \|\|\|+\|\|\| | |
| Sbjct: | 61 FFLSNLSFVDICFISTTVPKMLVNIQTQNNVITYAGCITQIYFFLLFVELDNFLLTIMAY 120 | |
| Query: | 386 DRFVAVCHPLHYMIIMNPHLCGLLVFVTWLIGVMTSLLHISLMMHLIFCKDFEIPHFFCE 565 | |
| | \|\|+\|\|+\|\|\|+\|\| +\|\|\| \|\|\| \|\| \|+\|++ \|+ +\| +\|+ \| \|\| \|\|\|\|+\|\|\| | |
| Sbjct: | 121 DRYVAICHPMHYTVIMNYKLCGFLVLVSWIVSVLHALFQSLMMLALPFCTHLEIPHYFCE 180 | |
| Query: | 566 LTYILQLACSDTFLNSTLIYFMTGVLGVFPLLGIIFSYSRIASSIRKMSSSGGKQKALST 745 | |
| | ++\|\| \|\|\| \|\|\| +\|\|\| +\| \|\| \|\| +\|\| +\| \|\|\| +\|\| \|\| \|\| \|\| | |
| Sbjct: | 181 PNQVIQLTCSDAFLNDLVIYFTLVLLATVPLAGIFYSYFKIVSSICAISSVHGKYKAFST 240 | |
| Query: | 746 CGSHLSVVSLFYGTGIGVHFTSAVTHSSQKISVASVMYTVVTPMLNPFIYSLRNKDVKGA 925 | |
| | \| \|\|\|\|\|\|\|\|\|\| \|\|+\|\|+ +\|\| +\|\|\| + \|\|\|\|\|\|\|\|\|\|\|+\|\|\|\|\|\|\|\|\|\|\|\|\| | |
| Sbjct: | 241 CASHLSVVSLFYCTGLGVYLSSAANNSSQASATASVMYTVVTPMVNPFIYSLRNKDVKSV 300 | |
| Query: | 926 LGSLL 940 | |
| | \| \| | |
| Sbjct: | 301 LKKTL 305 | |

Based on its relatedness to the GPCR superfamily proteins, the GPCR5 protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR6

GPCR6 includes a family of two similar nucleic acids and two similar proteins disclosed below. The disclosed nucleic acids encode GPCR, OR-like proteins.

### GPCR6a (AC011464_F)

The disclosed novel GPCR6 nucleic acid of 980 nucleotides is shown in Table 7A. A putative untranslated region upstream from the initiation codon and downstream from the termination codon are underlined in Table 7A, and the start and stop codons are in bold letters.

TABLE 7A
GPCR6a Nucleotide Sequence (SEQ ID NO:15)

TACTTCCAGAGGAATCACACCCATGGAACCAAGAAACCAAACCAGTGCATCTCAATTCATCCTCCTGGGA
CTCTCAGAAAAGCCAGAGCAGGAGACGCTTCTCTTTTCCCTGTTCTTCTGCATGTACCTGGTCATGGTCG
TGGGGAACCTGCTCATCATCCTGGCCATCAGCATAGACTCCCACCTCCACACCCCCATGTACTTCTTCCT
GGCCAACCTGTCCCTGGTTGATTTCTGTCTGGCCACCAACACCATCCCTAAGATGCTGGTGAGCCTTCAA
ACCGGGAGCAAGGCCATCTCTTATCCCTGCTGCCTGATCCAGATGTACTTCTTCCATTTCTTTGGCATCG
TGGACAGCGTCATAATCGCCATGATGGCTTATGACCGGTTCGTGGCCATCTGCCACCCATTGCACTACGC
CAAGATCATGAGCCTACGCCTCTGTCGCCTGCTGGTCGGCGCCCTCTGGGCGTTTTCCTGCTTCATCTCA
CTCACTCACATCCTCCTGATGGCCCGTCTCGTTTTCTGCGGCAGCCATGAGGTGCCTCACTACTTCTGCG
ACCTCACTCCCATCCTCCGACTTTCGTGCACGGACACCTCTGTGAATAGGATCTTCATCCTCATTGTGGC
AGGGATGGTGATAGCCACGCCCTTTGTCTGCATCCTGGCCTCCTATGCTCGCATCCTTGTGGCCATCATG
AAGGTCCCCTCTGCAGGCGGCAGGAAGAAAGCCTTCTCCACCTGCAGCTCCCACCTGTCTGTGGTTGCTC
TCTTCTATGGGACCACCATTGGCGTCTATCTGTGTCCCTCCTCGGTCCTCACCACTGTGAAGGAGAAAGC
TTCTGCGGTGATGTACACAGCAGTCACCCCCATGCTGAATCCCTTCATCTACAGCTTGAGGAACAGAGAC
CTGAAAGGGGCTCTCAGGAAGCTGGTCAACAGAAAGATCACCTCATCTTCCTGACCACCAGGACTCAGGA

The GPCR6a protein encoded by SEQ ID NO:15 has 313 amino acid residues, and is presented using the one-letter code in Table 7B (SEQ ID NO:16). The SignalP, Psort and/or Hydropathy profile for GPCR6a predict that GPCR6 has a signal peptide and is likely to be localized in the plasma membrane with a certainty of 0.6000. The most likely cleavage site is between amino acids 48 and 49. The predicted molecular weight is 34839.3Dal.

TABLE 7B
Encoded GPCR6a protein sequence (SEQ ID NO:16).

MEPRNQTSASQFILLGLSEKPEQETLLFSLFFCMYLVMVVGNLLIILAISIDSHLHTPMYFFLANLSLVDFCLAT
NTIPKMLVSLQTGSKAISYPCCLIQMYFFHFFGIVDSVIIAMMAYDRFVAICHPLHYAKIMSLRLCRLLVGALWA
FSCFISLTHILLMARLVFCGSHEVPHYFCDLTPILRLSCTDTSVNRIFILIVAGMVIATPFVCILASYARILVAI
MKVPSAGGRKKAFSTCSSHLSVVALFYGTTIGVYLCPSSVLTTVKEKASAVMYTAVTPMLNPFIYSLRNRDLKGA
LRKLVNRKITSSS

A GPCR6a polypeptide has 207 out of 305 (68%) amino acid residues identical to and 254 out of 305 similar to the 318 amino acid residue mus musculus odorant receptor protein S46 SPRTEMBL Accession No.: Q9WU93) (Table 7C).

TABLE 7C
BLASTX of GPCR6a against Odorant receptor clone
F3 - Rattus rattus (patp:AAR27867) (SEQ ID NO:33)

```
Top Previous Match Next Match
                        Length = 313
      Plus Strand HSPs:
  Score = 909 (320.0 bits), Expect = 2.3e−90, P = 2.3e−90
  Identities = 175/311 (56%), Positives = 223/311 (71%), Frame = +2
```

| | | |
|---|---|---|
| Query: | 23 MEPRNQTSASQFILLGLSEKPEQETLLFSLFFCMYLVMVVGNLLIILAISIDSHLHTPMY 202 | (SEQ ID NO:16) |
| | \| \|\|+\| ++\|+\|\|\|\|\| +\|+\|+ \|\|\| \|\| \|\|\| \|+\|\|\|\|\|\|\|\|\| \|\| \|\|\|\|\|\| | |
| Sbjct: | 1 MSSTNQSSVTEFLLLGLSRQPQQQQLLFLLFLIMYLATVLGNLLIILAIGTDSRLHTPMY 60 | (SEQ. ID NO:33) |
| Query: | 203 FFLANLSLVDFCLATNTIPKMLVSLQTGSKAISYPCCLIQMYFFHFFGIVDSVIIAMMAY 382 | |
| | \|\|\|+\|\|\| \|\| \| ++ \|+\|\|+\| + \|\|+\|\|\|+ \|\| \|+\|\| \|\| +\|+ ++\|+\|+\| | |
| Sbjct: | 61 FFLSNLSFVDVCFSSTTVPKVLANHILGSQAISFSGCLTQLYFLAVFGNMDNFLLAVMSY 120 | |
| Query: | 383 DRFVAICHPLHYAKIMSLRLCRLLVGALWAFSCFISLTHILLMARLVFCGSHEVPHYFCD 562 | |
| | \|\|\|\|\|\|\|\|\|\|\|\| \|+ +\|\| \|\|\| \| + \| \|\|\|\|\|\|\|\| \|\| + +\|\|+\|\|\| | |
| Sbjct: | 121 DRFVAICHPLHYTTKMTRQLCVLLVVGSWVVANMNCLLHILLMARLSFCADNMIPHFFCD 180 | |
| Query: | 563 LTPILRLSCTDTSVNRIFILIVAGMVIATPFVCILASYARILVAIMKVPSAGGRKKAFST 742 | |
| | \|\|+\|+\|\|\|+\|\| +\| + \|\| +\|+ \|\|\|\|\|\|\| \|\| \| \|+++\| \| \| \|+\|\|\| | |
| Sbjct: | 181 GTPLLKLSCSDTHLNELMILTEGAVVMVTPFVCILISYIHITCAVLRVSSPRGGWKSFST 240 | |
| Query: | 743 CSSHLSVVALFYGTTIGVYLCPSSVLTTVKEKASAVMYTAVTPMLNPFIYSLRNRDLKGA 922 | |
| | \| \|\|\|+\|\| \|\|\|\|\| \| \|\| \|\|\| ++ \|+\|\|\|\| \|\|\|\|\|\|\|\|\|\|\|\|\|\| \|+\| \| | |
| Sbjct: | 241 CGSHLAVVCLFYGTVIAVYFNPSSSHLAGRDMAAAVMYAVVTPMLNPFIYSLRNSDMKAA 300 | |
| Query: | 923 LRKLVNRKITS 955 | |
| | \|\|\|++ + \| | |
| Sbjct: | 301 LRKVLAMRFPS 311 | |

Single Nucleotide Polymorphisms (SNPs) were identified in a GPCR6a nucleic acid. The positions of the SNPs are listed in Table 7D.

TABLE 7D
cSNPs

| Base Position | | | Amino Acid |
|---|---|---|---|
| of cSNP | Wild Type | Variant | Change |
| 509 | A | G | none |

Based on its relatedness to the GPCR superfamily proteins, the GPCR6a protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR6b (dj1160k1_A_{—}1)

GPCR6a nucleic acid was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide GPCR6b. The nucleotide sequence for GPCR6b (SEQ ID NO:17) is presented in Table 6F. The nucleotide sequence differs from GPCR6a by 4 nucleotides changes at positions 103, 338, 508 and 559.

The disclosed novel GPCR6b nucleic acid of 944 nucleotides is shown in Table 7E. A putative untranslated region upstream from the initiation codon and downstream from the termination codon are underlined in Table 7E, and the start and stop codons are in bold letters.

TABLE 7E
GPCR6b Nucleotide Sequence (SEQ ID NO:17)

ACCCATGGAACCAAGAAACCAAACCAGTGCATCTCAATTCATCCTCCTGGGACTCTCAGAAAAGCCAGAG
CAGGAGACGCTTCTCTTTTCCCTGTTCTTCTGCATGTACCTGGTCATGGTCGTGGGGAACCTGCTCATCA
TCCTGGCCATCAGCATAGACTCCCACCTCCACACCCCCATGTACTTCTTCCTGGCCAACCTGTCCCTGGT
TGATTTCTGTCTGGCCACCAACACCATCCCTAAGATGCTGGTGAGCCTTCAAACCGGGAGCAAGGCCATC
TCTTATCCCTGCTGCCTGATCCAGATGTACTTCTTCCATTTCTTTGGCATCGTGGACAGCGTCATAATCG
CCATGATGGCTTATGACCGGTTCGTGGCCATCTGCCACCCGTTGCACTACGCCAAGATCATGAGCCTACG
CCTCTGTCGCCTGCTGGTCGGTGCCCTCTGGGCGTTTTCCTGCTTCATCTCACTCACTCACATCCTCCTG
ATGGCCCGTCTCGTTTTCTGCGGCAGCCATGAGGTGCCTCACTACTTCTGCGACCTCACTCCCATCCTCC
GACTTTCGTGCACGGACACCTCTGTGAATAGGATCTTCATCCTCATTGTGGTAGGGATGGTGATAGCCAC
GCCCTTTGTCTGCATCCTGGCCTCCTATGCTCGCATCCTTGTGGCCATCATGAAGGTCCCCTCTGCAGGC
GGCAGGAAGAAAGCCTTCTCCACCTGCAGCTCCCACCTGTCTGTGGTTGCTCTCTTCTATGGGACCACCA
TTGGCGTCTATCTGTGTCCCTCCTCGGTCCTCACCACTGTGAAGGAGAAAGCTTCTGCGGTGATGTACAC
AGCAGCCACCCCCATGCTGAATCCCTTCATCTACAGCTTGAGGAACAGAGACCTGAAAGGGGCTCTCAGG
AAGCTGGTCAACAGAAAGATCACCTCATCTTCCTGACCA

The GPCR6b protein encoded by SEQ ID NO:17 has 313 amino acid residues, and is presented using the one-letter code in Table 7F (SEQ ID NO:18). The SignalP, Psort and/or Hydropathy profile for GPCR6b predict that GPCR6b has a signal peptide and is likely to be localized at the plasma membrane with a certainty of 0.6000. The predicted cleavage site is between amino acids 48 and 49. The predicted molecular weight is 34839.3 Dal.

TABLE 7F
Encoded GPCR6b protein sequence (SEQ ID NO:18).

MEPRNQTSASQFILLGLSEKPEQETLLFSLFFCMYLVMVVGNLLIILAISIDSHLHTPMYFFLANLSLVD
FCLATNTIPKMLVSLQTGSKAISYPCCLIQMYFFHFFGIVDSVIIAMMAYDRFVAICHPLHYAKIMSLRL
CRLLVGALWAFSCFISLTHILLMARLVFCGSHEVPHYFCDLTPILRLSCTDTSVNRIFILIVVGMVIATP
FVCILASYARILVAIMKVPSAGGRKKAFSTCSSHLSVVALFYGTTIGVYLCPSSVLTTVKEKASAVMYTA
ATPMLNPFIYSLRNRDLKGALRKLVNRKITSSS

The full amino acid sequence of the protein of the invention was found to have 174 of 309 amino acid residues (56%) identical to, and 228 of 309 residues (73%) positive with, the 309 amino acid residue Gustatory Receptor 43—Rattus norvegicus (Rat) (Table 7G).

TABLE 7G
BLASTX of GPCR6b against Gustatory Receptor
43 - Rattusnorvegicus (Rat) (SEQ ID NO:34)

```
>ptnr:TREMBLNEW-ACC:BAA94424 GUSTATORY RECEPTOR
43 - Rattus norvegicus (Rat) , 311 aa.
Top Previous Match Next Match
                        Length = 311
    Plus Strand HSPs:
  Score = 932 (328.1 bits), Expect = 1.0e−92, P = 1.0e−92
  Identities = 174/309 (56%), Positives = 228/309 (73%), Frame = +2
```

| | | |
|---|---|---|
| Query: | 17 NQTSASQFILLGLSEKPEQETLLFSLFFCMYLVMVVGNLLIILAISIDSHLHTPMYFFLA 196 | (SEQ ID NO:18) |
| | \|\|+\| \|+\| \| \|+\| \|\|\|+ \|\|+ \|\| \|\|\|\|\| + \|\|+\|\|\|+\|\| \| \|\|\|\|\|\|\|\|\|\|\| | |
| Sbjct: | 3 NQSSVSEFFLQGISGFPEQQQLLYGLFLCMYLVTLTGNVLIIMAIGSDPHLHTPMYFFLA 62 | (SEQ ID NO:34) |
| Query: | 197 NLSLVDFCLATNTIPKMLVSLQTGSKAISYPCCLIQMYFFHFFGIVDSVIIAMMAYDRFV 376 | |
| | \|\|\| \| \| ++\|+ +\|\| ++\|\| \|\|\| \|\| \|\|\|\|\| \|\| +\|\| +\|+\|\|\|\|\|+\| | |
| Sbjct: | 63 NLSFADMGLISSTVTQMLFNVQTQRHTISYTGCLTQMYFFLMFGDLDSFFLAVMAYDRYV 122 | |
| Query: | 377 AICHPLHYAKIMSLRLCRLLVGALWAFSCFISLTHILLMARLVFCGSHEVPHYFCDLTPI 556 | |
| | \|\|\|\|\|\|\|\|+ \|\| ++\| \|++ \| + ++\|\|\| \|\|\|\|\|\| \|\| \|+ \|+\|\|\|+\|\|+ | |
| Sbjct: | 123 AICHPLHYSTIMRAKVCVLMLALCWVLTNIVALTHTLLMARLSFCVVGEIAHFFCDITPV 182 | |
| Query: | 557 LRLSCTDTSVNRIFILIVVGMVIATPFVCILASYARILVAIMKVPSAGGRKKAFSTCSSH 736 | |
| | \|+\|\|\|+\|\| \|\| + + + \| \|+ \|\|+\|\|+ \|\| \|+ \|\|++\| + \|\| \|\|\|\|\|\|\|\|\| | |
| Sbjct: | 183 LKLSCSDTYVNELMVFALGGTVLMLPFICIVISYIHIVFAILRVRTPGGGTKAFSTCSSH 242 | |
| Query: | 737 LSVVALFYGTTIGVYLCPSSVLTTVKEKASAVMYTAATPMLNPFIYSLRNRDLKGALRKL 916 | |
| | \| \|\| +\|\|\|\| \|\| \| \|\|++\| \|+ \|+\| \|\|\| \|\|\|\|\|\|\|\|\|\|\|\|\|+\|+\|\|\|\|++\| | |
| Sbjct: | 243 LCVVCVFYGTLFSAYLSPPSVVSTEKDIAAAAMYTVVTPMLNPFIYSLRNKDMKGALKRL 302 | |
| Query: | 917 V-NRKITSS 940 | |
| | + +\|+\| \|\| | |
| Sbjct: | 303 LFHRRILSS 311 | |

Based on its relatedness to the GPCR superfamily proteins, the GPCR6b protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR7 (CG53321-03)

The disclosed novel GPCR7 nucleic acid of 989 nucleotides is shown in Table 8A. A putative untranslated region upstream from the initiation codon and downstream from the termination codon are underlined in Table 8A, and the start and stop codons are in bold letters.

TABLE 8A
GPCR7 Nucleotide Sequence (SEQ ID NO:19)

| | |
|---|---|
| TTGACAAGATCAAGACTCATCAACAACATGAAAGCAGGAAACTTCTCAGACACTCCAGAA | 60 |
| TTCTTTCTCTTGGGATTGTCAGGGGATCCGGAGCTGCAGCCCATCCTCTTCATGCTGTTC | 120 |
| CTGTCCATGTACCTGGCCACAATGCTGGGGAACCTGCTCATCATCCTGGCCGTCAACTCT | 180 |
| GACTCCCACCTCCACACCCCCATGTACTTCCTCCTCTCTATCCTGTCCTTGGTCGACATC | 240 |
| TGTTTCACCTCCACCACGATGCCCAAGATGCTGGTGAACATCCAGGCACAGGCTCAATCC | 300 |
| ATCAATTACACAGGCTGCCTCACCCAAATCTGCTTTGTCCTGGTTTTTGTTGGATTGGAA | 360 |
| AATGGAATTCTGGTCATGATGGCCTATGATCGATTTGTGGCCATCTGTCACCCACTGAGG | 420 |
| TACAATGTCATCATGAACCCCAAACTCTGTGGGCTGCTGCTTCTGCTGTCCTTCATCGTT | 480 |
| AGTGTCCTGGATGCTCTGCTGCACACGTTGATGGTGCTACAGCTGACCTTCTGCATAGAC | 540 |
| CTGGAAATTCCCCACTTTTTCTGTGAACTAGCTCATATTCTCAAGCTCGCCTGTTCTGAT | 600 |
| GTCCTCATCAATAACATCCTGGTGTATTTGGTGACCAGCCTGTTAGGTGTTGTTCCTCTC | 660 |
| TCTGGGATCATTTTCTCTTACACACGAATTGTCTCCTCTGTCATGAAAATTCCATCAGCT | 720 |
| GGTGGAAAGTATAAAGCTTTTTCCATCTGCGGGTCACATTTAATCGTTGTTTCCTTGTTT | 780 |
| TATGGAACAGGGTTTGGGGTGTACCTTAGTTCTGGGGCTACCCACTCCTCCAGGAAGGGT | 840 |
| GCAATAGCATCAGTGATGTATACCGTGGTCACCCCCATGCTGAACCCACTCATTTACAGC | 900 |
| CTGAGAAACAAGGACATGTTGAAGGCTTTGAGGAAACTAATATCTAGGATACCATCTTTC | 960 |
| CATTGATGTCTCAGCTTCTTGGGCTTACA | 989 |

The GPCR7 protein encoded by SEQ ID NO:19 has 312 amino acid residues, and is presented using the one-letter code in Table 8B (SEQ ID NO:20). The SignalP, Psort and/or Hydropathy profile for GPCR7 predict that GPCR11 has a signal peptide and is likely to be localized in the plasma membrane with a certainty of 0.6000 The SignalP predicts a cleavage site at amino acid 51.

TABLE 8B
Encoded GPCR7 protein sequence (SEQ ID NO:20)

| | |
|---|---|
| MKAGNFSDTPEFFLLGLSGDPELQPILFMLFLSMYLATMLGNLLIILAVNSDSHLHTPMY | 60 |
| FLLSILSLVDICFTSTTMPKMLVNIQAQAQSINYTGCLTQICFVLVFVGLENGILVMMAY | 120 |
| DRFVAICHPLRYNVIMNPKLCGLLLLLSFIVSVLDALLHTLMVLQLTFCIDLEIPHFFCE | 180 |
| LAHILKLACSDVLINNILVYLVTSLLGVVPLSGIIFSYTRIVSSVMKIPSAGGKYKAFSI | 240 |
| CGSHLIVVSLFYGTGFGVYLSSGATHSSRKGAIASVMYTVVTPMLNPLIYSLRNKDMLKA | 300 |
| LRKLISRIPSFH | 312 |

A GPCR7 polypeptide has 201 of 302 (66%) amino acid residues identical to and 241 of 302 (79%) similar to the 309 amino acid residue STREMBL-ACC:07.6100 protein from Homo sapiens(Human) (Table 8C)

```
TABLE 8C
BLASTX of GPCR7 against STREMBL-ACC:07.6100 protein from
Homo sapiens (Human) (SEQ ID NO:35)
>ptnr:SPTREMBL-ACC:076100 BC85395_3 - Homo sapiens (Human), 309 aa.
                        Length = 309
  Score = 1033 (363.6 bits), Expect = 4.3e−104, P = 4.3e−104
  Identities = 201/302 (66%), Positives = 241/302 (79%)
```

| | | |
|---|---|---|
| Query: | 1 MKAGNFSDTPEFFLLGLSGDPELQPILFMLFLSMYLATMLGNLLIILAVNSDSHLHTPMY 60 | (SEQ ID NO:20) |
| | \|\|+ \| + \|\| \|\|\|+\| +\|\|\|\| \|\| \|\|\|\|\|\|\| \|+\|\|\|\|\|\|\|\|\| \|\|\|\|\|\|\|\|\|\| | |
| Sbjct: | 1 MKSWNNTIILEFLLLGISEEPELQAFLFGLFLSMYLVTVLGNLLIILATISDSHLHTPMY 60 | (SEQ ID NO:35) |
| Query: | 61 FLLSILSLVDICFTSTTMPKMLVNIQAQAQSINYTGCLTQICFVLVFVGLENGILVMMAY 120 | |
| | \| \|\| \|\| \|\|\|\|\| \|\|\|+\|\|\|\|\|\|\|\| + \| \| \|\|+\|\|+\|\| \|+\|\|\|\|+\| +\| +\|\|\| | |
| Sbjct: | 61 FFLSNLSFVDICFVSTTVPKMLVNIQTHNKVITYAGCITQMCFFLLFVGLDNFLLTVMAY 120 | |
| Query: | 121 DRFVAICHPLRYNVIMNPKLCGLLLLLSFIVSVLDALLHTLMVLQLTFCIDLEIPHFFCE 180 | |
| | \|\|\|\|\|\|\|\|\|\| \| \|\|\|\|\|+\|\|\|\|\|+\| \|+\|+\|\|\|+++\| +\|\|\|\| \| \|\| +\|\|\|\|\|\|\|\| | |
| Sbjct: | 121 DRFVAICHPLHYMVIMNPQLCGLLVLASWIMSVLNSMLQSLMVLPLPFCTHMEIPHFFCE 180 | |
| Query: | 181 LAHILKLACSDVLINNILVYLVTSLLGVVPLSGIIFSYTRIVSSVMKIPSAGGKYKAFSI 240 | |
| | + ++ \|\|\|\|\| +\|+\|++\| +\|\|\| \|\|+\|\|++\|\|++\|\|\|\|+ \| \|\| \|\|\|\|\|\|\| | |
| Sbjct: | 181 INQVVHLACSDTFLNDIVMYFAVALLGGGPLTGILYSYSKIVSSIRAISSAQGKYKAFST 240 | |
| Query: | 241 CGSHLIVVSLFYGTGFGVYLSSGATHSSRKGAIASVMYTVVTPMLNPLIYSLRNKDMLKA 300 | |
| | \| \|\|\| \|\|\|\|\|\|\|\| \|\|\|\|\|\| \|\|\|+\| \|\| \|\|\|\|\|\|\|\|\|\|\|\|\|\| \|\|\|\|\|\|\| + \| | |
| Sbjct: | 241 CASHLSVVSLFYGTCLGVYLSSAATHNSHTGAAASVMYTVVTPMLNPFIYSLRNKHIKGA 300 | |
| Query: | 301 LR 302 | |
| | ++ | |
| Sbjct: | 301 MK 302 | |

### SNPs and cSNPS

Single nucleotide polymorphism analysis is detailed in Example 2. As is shown in Table 8D, in the following positions, one or more consensus positions (Cons. Pos.) of the nucleotide sequence have been identified as SNPs. “Depth represents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 8D
SNPs and cSNPS

| | | | |
|---|---|---|---|
| Cons. Pos.: 114 | Depth: 24 | Change: A > G | Putative Allele Freq.: 0.083 |
| Cons. Pos.: 222 | Depth: 24 | Change: T > C | Putative Allele Freq.: 0.083 |
| Cons. Pos.: 267 | Depth: 24 | Change: C > T | Putative Allele Freq.: 0.083 |
| Cons. Pos.: 317 | Depth: 23 | Change: A > G | Putative Allele Freq.: 0.087 |
| Cons. Pos.: 397 | Depth: 28 | Change: C > T | Putative Allele Freq.: 0.107 |
| Cons. Pos.: 414 | Depth: 28 | Change: T > C | Putative Allele Freq.: 0.071 |
| Cons. Pos.: 774 | Depth: 19 | Change: T > C | Putative Allele Freq.: 0.263 |

Based on its relatedness to the GPCR superfamily proteins, the GPCR7 protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR8

GPCR8 includes a family of two similar nucleic acids and two similar proteins disclosed below. The disclosed nucleic acids encode GPCR, OR-like proteins.

### GPCR8a (dj11601k1_A)

The disclosed novel GPCR8a nucleic acid of 1364 nucleotides is shown in Table 9A. A putative untranslated region upstream from the initiation codon and downstream from the termination codon are underlined in Table 9A, and the start and stop codons are in bold letters.

TABLE 9A
GPCR8a Nucleotide Sequence

| | |
|---|---|
| GCCCCATGGAGTCCTCACCCATCCCCCAGTCATCAGGGAACTCTTCCACTTTGGGGAGGGTCCCTCAAAC | (SEQ ID NO:21) |
| CCCAGGTCCCTCTACTGCCAGTGGGGTCCCCGAGGTGGGGCTACGCGATGTTGCTTCGCAATCTGTGGCC | |
| CTCTTCTTCATGCTCCTGCTGGACTTGACTGCTGTGGCTGGCAATGCCGCTGTGATGGCCGTGATCGCCA | |
| AGACGCCTGCCCTCCGAAAATTTGTCTTCGTCTTCCACCTCTGCCTGGTGGACCTGCTGGCTGCCCTGAC | |
| CCTCATGCCCCTGGCCATGCTCTCCAGCTCTGCCCTCTTTGACCACGCCCTCTTTGGGGAGGTGGCCTGC | |
| CGCCTCTACTTGTTTCTGACCGTGTGCTTTGTCAGCCTGGCCATCCTCTCCGTGTCAGCCATCAATGTGG | |
| AGCGCTACTATTACGTAGTCCACCCCATGCGCTACGAGGTGCGCATGACGCTGGGGCTGGTGGCCTCTGT | |
| GCTGGTGCGTGTGTGGGTGAAGGCCTTGGCCATGGCTTCTGTGCCAGTGTTGGGAAGGGTCTCCTGGGAG | |
| GAAGGAGCTCCCAGTGTCCCCCCAGGCTGTTCACTCCACTGGAGCCACAQTGCCTACTGCCAGCTTTTTG | |
| TGGTGGTCTTTGCTGTCCTTTACTTTCTGTTGCCCCTGCTCCTCATACTTGTGGTCTACTGCAGCATGTT | |
| CCGAGTGGCCCGCGTGGCTGCCATGCAGCACGGGCCGCTGCCCACGTGGATGGAGACACCCCGGCAACGC | |
| TCCGAATCTCTCAGCAGCCGCTCCACGATGGTCACCAGCTCGGGGGCCCCCCAGACCACCCCACACCGGA | |
| CGTTTGGGGGAGGGAAAGCAGCAGTGGTTCTCCTGGCTGTGGGGGGACAGTTCCTGCTCTGTTGGTTGCC | |
| GTCACCTGGATTGGCTACTTTTGCTTCACTTCCAACCCTTTCTTCTATGGATGTCTCAACCGGCAGATCC | |
| CGGGGGAGCTCAGCAAGCAGTTTGTCTGCTTCTTCAAGCCAGCTCCAGAGGAGGAGCTGAGGCTGCCTAG | |
| CCGGGAGGGCTCCATTGAGGAGAACTTCCTGCAGTTCCTTCAGGGGACTGGCTGTCCTTCTGAGTCCTGG | |
| GTTTCCCGACCCCTACCCAGCCCCAAGCAGGAGCCACCTGCTGTTGACTTTCGAATCCCAGGCCAGATAG | |
| CTGAGGAGACCTCTGAGTTCCTGGAGCAGCAACTCACCAGCGACATCATCATGTCAGACAGCTACCTCCG | |
| TCCTGCCGCCTCACCCCGGCTGGAGTCATGATGG | |

The GPCR8 protein encoded by SEQ ID NO:21 has 451 amino acid residues, and is presented using the one-letter code in Table 9B (SEQ ID NO:22). The SignalP, Psort and/or Hydropathy profile for GPCR8 predict that GPCR8 has a signal peptide and is likely to be localized at the plasma membrane with a certainty of 0.6000. The SignalP shows that the protein has a signal peptide with most likely cleavage site between positions 61 and 62. The predicted molecular weight is 49291.8 Dal.

TABLE 9B
Encoded GPCR8a protein sequence.

| | |
|---|---|
| MESSPIPQSSGNSSTLGRVPQTPGPSTASGVPEVGLRDVASESVALFFMLLLDLTAVAGNAAVMAVIAKT | (SEQ ID NO:22) |
| PALRKFVFVFHLCLVDLLAALTLMPLAMLSSSALFDHALFGEVACRLYLFLSVCFVSLAILSVSAINVER | |
| YYYVVHPMRYEVRMTLGLVASVLVGVWVKALAMASVPVLGRVSWEEGAPSVPPGCSLQWSHSAYCQLFVV | |
| VFAVLYFLLPLLLILVVYCSMFRVARVAAMQHGPLPTWMETPRQRSESLSSRSTMVTSSGAPQTTPHRTF | |
| GGGKAAVVLLAVGGQFLLCWLPYFSFHLYVALSAQPISTGQVESVVTWIGYFCFTSNPFFYGCLNRQIRG | |
| ELSKQFVCFFKPAPEEELRLPSREGSIEENFLQFLQGTGCPSESWVSRPLPSPKQEPPAVDFRIPGQIAE | |
| ETSEFLEQQLTSDIIMSDSYLRPAASPRLES | |

The full amino acid sequence of the protein of the invention was found to have 247 out of 252 amino acid residues (98%) identical to, and 249 of 252 residues (98%) positive with the 252 amino acid residue Rabbit G-protein coupled receptor protein portion—Orycctolagus cuniculus (patp: AAR91232) (Table 9C).

TABLE 9C
BLASTX of GPCR8a against Rabbit G-protein coupled receptor
protein portion - Orycctolagus cuniculus (patp: AAR91232) (SEQ ID NO:36)

```
Top Previous Match Next Match
                          Length = 252
    Plus Strand HSPs:
  Score = 1276 (449.2 bits), Expect = 3.0e−129, P = 3.Oe−129
  Identities = 247/252 (98%), Positives = 249/252 (98%), Frame = +3
```

| | | | | |
|---|---|---|---|---|
| Query: | 258 | VDLLAALTLMPLAMLSSSALFDHALFGEVACRLYLFLSVCFVSLAILSVSAINVERYYYV | 43 | (SEQ ID NO:22) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbj ct: | 1 | VDLLAALTLMPLAMLSSSALFDHALFGEVACRLYLFLSVCFVSLAILSVSAINVERYYYV | 60 | (SEQ ID NO:36) |
| Query: | 438 | VHPMRYEVRMTLGLVASVLVGVWVKALAMASVPVLGRVSWEEGAPSVPPGCSLQWSHSAY | 61 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbj ct: | 61 | VHPMRYEVRMKLGLVASVLVGVWVKALAMASVPVLGRVSWEEGPPSVPPGCSLQWSHSAY | 12 | |
| Query: | 618 | CQLFVVVFAVLYFLLPLLLILVVYCSMFRVARVAAMQHGPLPTWMETPRQRSESLSSRST | 79 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbj ct: | 121 | CQLFVVVFAVLYFLLPLLLILVVYCSMFRVARVAAMQHGPLPTWMETPRQRSESLSSRST | 18 | |
| Query: | 798 | MVTSSGAPQTTPHRTFGGGKAAVVLLAVGGQFLLCWLPYFSFHLYVALSAQPISTGQVES | 97 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbj ct: | 181 | MVTSSGAPQTTPHRTFGGGKAAVVLLAVGGQFLLCWLPYFSFHLYVALSAQPIAAGQVEN | 24 | |

| | | | |
|---|---|---|---|
| Query: | 978 | VVTWIGYFCFTS | 1013 |
| | | \|\|\|\|\|\|\|\|\|\|\|\| | |
| Sbjct: | 241 | VVTWIGYFCFTS | 252 |

Based on its relatedness to the GPCR superfamily proteins, the GPCR8a protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR8b (CG5743-02)

GPCR8a nucleic acid was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide GPCR8b. The nucleotide sequence for GPCR8b (SEQ ID NO:23) is presented in Table 9D. The nucleotide sequence differs from GPCR8a by 2 nucleotide changes at positions 573 and 1245.

The disclosed novel GPCR8b nucleic acid of 1364 nucleotides is shown in Table 9D. A putative untranslated region upstream from the initiation codon and downstream from the termination codon are underlined in Table 9D, and the start and stop codons are in bold letters.

TABLE 9D
GPCR8b Nucleotide Sequence

| | | |
|---|---|---|
| GCCCCATGCACTCCTCACCCATCCCCCAGTCATCAGGGAACTCTTCCACTTTGCGGAGGG | 60 | (SEQ ID NO:23) |
| TCCCTCAAACCCCAGGTCCCTCTACTGCCAGTGGGGTCCCGGAGGTGGGGCTACGGGATG | 120 | |
| TTGCTTCGGAATCTGTCGCCCTCTTCTTCATGCTCCTGCTGGACTTGACTGCTCTGGCTG | 180 | |
| GCAATGCCGCTGTGATGGCCGTGATCGCCAAGACGCCTGCCCTCCGAAAATTTGTCTTCG | 240 | |
| TCTTCCACCTCTGCCTGGTCGACCTGCTGGCTGCCCTGACCCTCATGCCCCTGGCCATGC | 300 | |
| TCTCCAGCTCTGCCCTCTTTGACCACGCCCTCTTTGGGGAGGTGGCCTGCCGCCTCTACT | 360 | |
| TGTTTCTGAGCGTGTGCTTTGTCACCCTGGCCATCCTCTCGGTGTCAGCCATCAATGTGG | 420 | |
| AGCGCTACTATTACGTAGTCCACCCCATGCGCTACGAGGTGCGCATGACGCTGGGGCTGC | 480 | |
| TGGCCTCTGTGCTGGTGGGTGTGTCGCTGAAGGCCTTGGCCATGGCTTCTGTGCCAGTGT | 540 | |
| TGGGAAGGGTCTCCTGGGAGGAAGGAGCTCCTAGTGTCCCCCCAGGCTGTTCACTCCAGT | 600 | |
| GGAGCCACAGTGCCTACTGCCAGCTTTTTGTGGTGGTCTTTGCTGTCCTTTACTTTCTGT | 660 | |
| TGCCCCTGCTCCTCATACTTGTGGTCTACTGCAGCATGTTCCGAGTGGCCCGCGTGGCTG | 720 | |
| CCATGCAGCACGGGCCGCTGCCCACGTGGATGGAGACACCCCGGCAACGCTCCGAATCTC | 780 | |
| TCAGCAGCCGCTCCACGATGGTCACCAGCTCGGGGGCCCCCCAGACCACCCCACACCGGA | 840 | |
| CGTTTGGGGGAGGGAAAGCAGCAGTGGTTCTCCTGGCTGTGGGGGGACAGTTCCTGCTCT | 900 | |
| GTTGGTTGCCCTACTTCTCTTTCCACCTCTATGTTGCCCTGAGTGCTCAGCCCATTTCAA | 860 | |
| CTGGGCACGTGGACAGTGTCCTCACCTGGATTGGCTACTTTTGCTTCACTTCCAACCCTT | 1020 | |
| TCTTCTATGGATGTCTCAACCGGCAGATCCGGCGCCAGCTCAGCAAGCACTTTGTCTGCT | 1080 | |
| TCTTCAAGCCAGCTCCAGAGGAGCAGCTGAGGCTGCCTAGCCGGGAGGGCTCCATTGAGG | 1140 | |
| AGAACTTCCTGCAGTTCCTTCAGGGGACTGGCTCTCCTTCTGAGTCCTGGGTTTCCCGAC | 1200 | |
| CCCTAGCCAGCCCCAAGCAGGAGCCACCTGCTGTTGACTTTCGAGTCCCAGCCCAGATAG | 1280 | |
| CTGAGGAGACCTCTGAGTTCCTGGAGCAGCAACTCACCAGCGACATCATCATGTCAGACA | 1320 | |

| | |
|---|---|
| GCTACCTCCGTCCTGCCGCCTCACCCCGGCTGGAGTCATGATGG | 1364 |

The GPCR8b protein encoded by SEQ ID NO:23 has 309 amino acid residues, and is presented using the one-letter code in Table 9E (SEQ ID NO:24). The SignalP, Psort and/or Hydropathy profile for GPCR8b predict that GPCR8b has a signal peptide and is likely to be localized at the plasma membranse with a certainty of 0.6000. The SignalP predicts a cleavage site at the sequence between amino acids 61 and 62.

TABLE 9E
Encoded GPCR8b protein segnence

| | | |
|---|---|---|
| MESSPIPQSSGNSSTLGRVPQTPGPSTASGVPEVGLRDVASESVALFFMLLLDLTAVAGN | 60 | (SEQ ID NO:24) |
| AAVMAVIAKTPALRKFVFVFHLCLVDLLAALTLMPLAMLSSSALFDHALFGEVACRLYLF | 120 | |
| LSVCFVSLAILSVSAINVERYYYVVHPMRYEVRMTLGLVASVLVGVWVKALAMASVPVLG | 130 | |
| RVSWEEGAPSVPPGCSLQWSHSAYCQLFVVVFAVLYFLLPLLLILVVYCSMFRVARVAAM | 240 | |
| QHGPLPTWMETPRQRSESLSSRSTMVTSSGAPQTTPHRTFGGGKAAVVLLAVGGQFLLCW | 300 | |
| LPYFSFHLYVALSAQPISTGQVESVVTWIGYFCFTSNPFFYGCLNRQIRGELSKQFVCFF | 360 | |
| KPAPEEELRLPSREGSIEENFLQFLQGTGCPSESWVSRPLPSPKQEPPAVDFRVPGQIAE | 420 | |

| | |
|---|---|
| ETSEFLEQQLTSDIIMSDSYLRPAASPRLES | 451 |

A GPCR8b polypeptide has 181 of 422 (42%) amino acid residues identical to and 266 of 422 (63%) similar to the 428 amino acid residue SWISSPROT-ACC:Q91178 protein from Oryzias latipes (Medaka fish) (Table 9F).

TABLE 9F
BLASTP of GPCR8b against GPCR protein from Oryzias latipes
(Medaka fish) (ptnr:SPTREMBL-ACC: Q91178) (SEQ ID NO:37)

```
>ptnr:SWISSPROT-ACC:Q91178 PROBABLE G PROTEIN-COUPLED RECEPTOR - Oryzias
                        latipes (Medaka fish), 428 as (fragment).
                        Length = 428
  Score = 822 (289.4 bits), Expect = 9.6e−82, P = 9.8e−82
  Identities = 181/422 (42%), Positives = 266/422 (63%)
```

| | | | | |
|---|---|---|---|---|
| Query: | 2 | ESSPIPQSSGNSSTLGRVPQTPGPSTASGVPEVGL----RDVASESVALFFMLLLDLTAV | 57 | (SEQ ID NO:24) |
| | | ++\|\|+ \| + \| \| \|+ \|+\|\|+ + + \|\| \|+ \|+\| \|+ | | |
| Sbjct: | 5 | KTSPMITSDHSISNFSTGLFGPHPTVP---PDVGVVTSSQSQMKDLFGLFCMVTLNLIAL | 61 | (SEQ ID NO:37) |
| Query: | 58 | AGNAAVMAVIAKTPALRKFVFVFHLCLVDLLAALTLMPLAMLSSSALFDHALFGEVACRL | 117 | |
| | | \| \|\| \|\|+ \| \|+\|\| \|\| \|\|\| \|\|+\| \|+ \|\|\|\| ++\|\|\| \| +\| + \|++ | | |
| Sbjct: | 62 | LANTCVMVAIARAPHLRKFAFVCHLCAVDVLCAILLMPLGIISSSPFFGTVVFTILECQV | 121 | |
| Query: | 118 | YLFLSVCFVSLAILSVSAINVERYYYVVHPNRYEVRMTLGLVASVLVGVWVKALAMASVP | 177 | |
| | | \|+\|\|+\| + \|+\|\|+++\|\|+\|\|\|\|+\|+\|\|\|\|\|\|\|\|+\|\|+ \|\| \|++ +\| \|+\| +\| \| | | |
| Sbjct: | 122 | YIFLNVFLIWLSILTITAISVERYFYIVHPMRYEVKMTINLVIGVMLLIWFRSLLLALVT | 181 | |
| Query: | 178 | VLGRVSWEEGAPSVPPCCSLQWSHSAYCQLFVVVFAVLYFLLPLLLILVVYCSMFRVARV | 237 | |
| | | + \| + + \|\|\| \|\|\| +\| \|+\| \|+ \|\| \|+++\| \|\| ++++\|\|\| | | |
| Sbjct: | 182 | LFGWPPYCHQSSIAASHCSLHASHSRLRGVFAVLFCVICFLAPVVVTFSVYSAVYRVARS | 241 | |
| Query: | 238 | AAMQHGP-LPTWME-TP-RQRSESLSSRSTMVTSSGAPQT-TPHRTFGGGKAAVVLLAVG | 293 | |
| | | \|\|+\| \| +\|\|\| + +\| + \|\|+\|++\|++\|++\|+ \|\| +\| \| \| \|\|\|\|\|+ \| + | | |
| Sbjct: | 242 | AALQQVPAVPTWAOASPARDRSOSINEQTTIITTRTLPQRLSPERAFSGGRAALTLAFIV | 301 | |
| Query: | 294 | GQFLLCWLPYFSFHLYVALSAQPISTGQVESVVTWIGYFCFTSNPFFYGCLNRQIRGELS | 353 | |
| | | \|\|\|\|+\|\|\|\|+\| \|\|\| ++\|+ \| \| +\| \| \|+ \| \| \|\| \|\|\| \|\|\|\|\|\| \|\| | | |
| Sbjct: | 302 | GQFLVCWLPFFIFHLQNSLTOSMKSPGOLEEAVNWLAYSSFAVNPSFYGLLNRQIRDELV | 361 | |
| Query: | 354 | K-QFVCFFKPAPEEELRLPSREGSIEENFLQFLQGTGCPSESWVSRPLPSPKQ-EPPAVD | 411 | |
| | | \| + \| +\| \|+ \| \|\|\| +\|\|\|\|\|\|+\| \| \|\|+ \| +\|+ \| \| | | |
| Sbjct: | 362 | KFRRCCVTQPV---EIGPSSLEGSPQENFLQFIQRTSSSSETHPSFANSNPRMMENQA-- | 416 | |

| | | | |
|---|---|---|---|
| Query: | 412 | FRVPGQIAEE | 421 |
| | | ++\|\|\|\| \|\| | |
| Sbjct: | 417 | BRIPOQIPEE | 426 |

Quantitative expression of GPCR8b was assessed as disclosed in Example 3E.

Based on its relatedness to the GPCR superfamily proteins, the GPCR8b protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCR9 (SC80023385)

The disclosed novel GPCR9 that is comprised of a nucleic acid of 1560 nucleotides is shown in Table 10A and was identified on chrmosome 12p13.3. A putative untranslated region upstream from the initiation codon and downstream from the termination codon are underlined in Table 10A, and the start and stop codons are in bold letters.

TABLE 10A
GPCR9 Nucleotide Sequence

| | |
|---|---|
| GAGAAAAGATTCAGAAGGCCTGCCAGTGGAGCTAAACATTTGTGTGTGCAGCCCTGTCTCTGTATAACTT | (SEQ ID NO:25) |
| CCGGCTTGCCTTCCTATTCCAGGTCTCTGCTGCTGATGAAGCTGTGACCAAACGCACCCAACCCTTGGCA | |
| GCCATCTGTCCCTGCAGCCATAGCCCACATTCCCATGACCTCCCTCTGCTTGTTTTGGGACCATGTCTGT | |
| ACAGCCTCTAGGCCCCAGCCCCGGAGGTGAATGCCATGCCATGATTCTGGTGTGCTCCATGGCATCCCCA | |
| GCCTAGCTCCCAATCCCACTTTGGCACGATGTTAGCCAACAGCTCCTCAACCAACAGTTCTGTTCTCCCG | |
| TGTCCTGACTACCGACCTACCCACCGCCTGCACTTGGTGGTCTACAGCTTGGTGCTGGCTGCCGGGCTCC | |
| CCCTCAACGCGCTAGCCCTCTGGGTCTTCCTGCGCGCGCTGCGCGTGCACTCGGTGGTGAGCGTGTACAT | |
| GTGTAACCTGGCGGCCAGCGACCTGCTCTTCACCCTCTCGCTGCCCGTTCGTCTCTCCTACTACGCACTG | |
| CACCACTGGCCCTTCCCCGACCTCCTGTGCCAGACGACGGGCGCCATCTTCCAGATGAACATGTACGGCA | |
| GCTGCATCTTCCTGATGCTCATCAACGTGGACCGCTACGCCGCCATCGTGCACCCGCTGCGACTGCGCCA | |
| CCTGCGGCGGCCCCGCGTGGCGCGGCTGCTCTGCCTGGGCGTGTGGGCGCTCATCCTGGTGTTTGCCGTG | |
| CCCGCCGCCCGCGTGCACAGGCCCTCGCGTTGCCGCTACCGGGACCTCGAGGTGCGCCTATGCTTCGAGA | |
| GCTTCAGCGACGAGCTGTGGAAAGGCAGGCTGCTGCCCCTCGTGCTGCTGGCCGAGGCGCTGGGCTTCCT | |
| GCTGCCCCTGGCGGCGGTGGTCTACTCGTCGGGCCGAGTCTTCTGGACGCTGGCGCGCCCCGACGCCACG | |
| CAGAGCCAGCGGCGGCGGAAGACCGTGCGCCTCCTGCTGGCTAACCTCGTCATCTTCCTGCTGTGCTTCG | |
| TGCCCTACAACAGCACGCTGGCGGTCTACGGGCTGCTGCGGAGCAAGCTGGTGGCGGCCAGCGTGCCTGC | |
| CCGCGATCGCGTGCGCGGGGTGCTGATGGTGATGGTGCTGCTGGCCGGCGCCAACTGCGTGCTGGACCCG | |
| CTGGTGTACTACTTTAGCGCCGAGGGCTTCCGCAACACCCTGCGCGGCCTGGGCACTCCGCACCGGGCCA | |
| GGACCTCGGCCACCAACGGGACGCGGGCGGCGCTCGCGCAATCCGAAAGGTCCGCCGTCACCACCGACGC | |
| CACCAGGCCGGATGCCGCCAGTCAGGGGCTGCTCCGACCCTCCGACTCCCACTCTCTGTCTTCCTTCACA | |
| CAGTGTCCCCAGGATTCCGCCCTCTGAACACACATGCCATTGCGCTGTCCGTGCCCGACTCCCAACGCCT | |
| CTCGTTCTGGGAGGCTTACAGGGTGTACACACAAGAAGGTGGGCTGGGCACTTGGACCTTTGGGTGGCAA | |
| TTCCAGCTTAGCAACGCAGA | |

The GPCR9 protein encoded by SEQ ID NO:25 has 372 amino acid residues, and is presented using the one-letter code in Table 10B (SEQ ID NO:26). The SignalP, Psort and/or Hydropathy profile for GPCR10 predict that GPCR10 has a signal peptide and is likely to be localized in the plasma membrane with a certainty of 0.6000. The SignalP predicts a cleavage site between amino acids 58 and 59.

TABLE 10B
Encoded GPCR9protein sequence

| | |
|---|---|
| MLANSSSTNSSVLPCPDYRPTHRLHLVVYSLVLAAGLPLNALALWVFLRALRVHSVVSVYMCNLAASDLLF | (SEQ ID NO:26) |
| TLSLPVRLSYYALHHWPFPDLLCQTTGAIFQMNMYGSCIFLMLINVDRYAAIVHPLRLRHLRRPRVARLLC | |
| LGVWALILVFAVPAARVHRPSRCRYRDLEVRLCFESFSDELWKGRLLPLVLLAEALGFLLPLAAVVYSSGR | |
| VFWTLARPDATQSQRRRKTVRLLLANLVIFLLCFVPYNSTLAVYGLLRSKLVAASVPARDRVRGVLMVMVL | |
| LAGANCVLDPLVYYFSAEGFRNTLRGLGTPHRARTSATNGTRAALAQSERSAVTTDATRPDAASQGLLRPS | |
| DSHSLSSFTQCPQDSAL | |

A GPCR9 polypeptide has 372 of 372 amino acid residues (100%) identical to, and 372of 372 residues (100%) positive with, the 372 amino acid residue human P2Y-like transmembrane receptor AXOR17—Homo sapiens (patp: AAB08621) (Table 10C).

TABLE 10C
BLASTX of GPRC9 against human P2Y-like trausmembrane
receptor AXOR17 - Homo sapiens (patp: AAB08621) (SEQ ID NO:38)

```
Top Previous Match Next Match
                      Length = 372
    Plus Strand HSPs:
  Score 1902 (669.5 hits), Expect = l.4e−195, P = l.4e−195
  Identities = 372/372 (100%), Positives = 372/372 (100%), Frane = +3
```

| | | | | |
|---|---|---|---|---|
| Query: | 309 | MLANSSSTNSSVLPCPDYRPTHRLHLVVYSLVLAAGLPLNALALWVFLRALRVHSVVSVY | 48 | (SEQ ID NO:26) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 3 | MLANSSSTNSSVLPCPDYRPTHRLHLVVYSLVLAAGLPLNALALWVFLRALRVHSVVSVY | 60 | (SEQ ID NO:38) |
| Query: | 489 | MCNLAASDLLFTLSLPVRLSYYALHHWPFPDLLCQTTGAIFQMNMYGSCIFLMLINVDRY | 66 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | MCNLAASDLLFTLSLPVRLSYYALHHWPFPDLLCQTTGAIFQMNMYGSCIFLMLINVDRY | 12 | |
| Query: | 669 | AAIVHPLRLRHLRRPRVARLLCLGVWALILVFAVPAARVHRPSRCRYRDLEVRLCFESFS | 84 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | AAIVHPLRLRHLRRPRVARLLCLGVWALILVFAVPAARVHRPSRCRYRDLEVRLCFESFS | 18 | |
| Query: | 849 | DELWKGRLLPLVLLAEALGFLLPLAAVVYSSGRVFWTLARPDATQSQRRRKTVRLLLANL | 10 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | DELWKGRLLPLVLLAEALGFLLPLAAVVYSSGRVFWTLARPDATQSQRRRKTVRLLLANL | 24 | |
| Query: | 1029 | VIFLLCFVPYNSTLAVYGLLRSKLVAASVPARDRVRGVLMVMVLLAGANCVLDPLVYYFS | 12 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 241 | VIFLLCFVPYNSTLAVYGLLRSKLVAASVPARDRVRGVLMVMVLLAGANCVLDPLVYYFS | 30 | |
| Query: | 1209 | AEGFRNTLRGLGTPHRARTSATNGTRAALAQSERSAVTTDATRPDAASQGLLRPSDSHSL | 13 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | AEGFRNTLRGLGTPHRARTSATNGTRAALAQSERSAVTTDATRPDAASQGLLRPSDSHSL | 36 | |

| | | | |
|---|---|---|---|
| Query: | 1389 | SSFTQCPQDSAL | 1424 |
| | | \|\|\|\|\|\|\|\|\|\|\|\| | |
| Sbjct: | 361 | SSFTQCPQDSAL | 372 |

GPCR9 homology with other sequences (Patp results) is shown in Table 10D.

TABLE 10D
Patp alignments of GPCR9

| | | | | Smallest | |
|---|---|---|---|---|---|
| | | | | Sum | |
| Sequences producing | | Reading | High | Probability | |
| High-scoring Segment Pairs: | | Frame | Score | P(N) | N |
| patp:B0861 | Human P2Y-like 7 | +3 | 1902 | 1.4e−195 | 1 |
| | transmembrane receptor | | | | |
| patp:Y71292 | Human orphan G-protein | +3 | 1902 | 1.4e−195 | 1 |
| | coupled receptor | | | | |
| patp:B0289 | Human P2Y-like 7 | +3 | 1902 | 1.4e−195 | 1 |
| | transmembrane receptor | | | | |
| patp:546598 | Human P2YLi Protein | +3 | 1902 | 1.4e−195 | 1 |
| patp:B61612 | Human protein HPO3378 | +3 | 1902 | 1.4e−195 | 1 |
| patp:Y72590 | Human G-protein | +3 | 1902 | 1.4e−195 | 1 |
| | coupled receptor.ICSR-1 | | | | |
| patp:Y79564 | Human G-protein | +3 | 1890 | 2.6e−194 | 1 |
| | coupled receptor | | | | |

Quantitative expression of GPCR9 was assessed as disclosed in Example 3F.

Based on its relatedness to the GPCR superfamily proteins, the GPCR9 protein is a novel member of the GPCR protein family. The discovery of molecules related to GPCR satisfies a need in the art by providing new diagnostic or therapeutic compositions useful in the treatment of disorders associated with alterations in the expression of members of GPCR-like proteins.

### GPCRX Nucleic Acids and Polypeptides

One aspect of the invention pertains to isolated nucleic acid molecules that encode GPCRX polypeptides or biologically active portions thereof. Also included in the invention are nucleic acid fragments sufficient for use as hybridization probes to identify GPCRX-encoding nucleic acids (e.g., GPCRX mRNAs) and fragments for use as PCR primers for the amplification and/or mutation of GPCRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An GPCRX nucleic acid can encode a mature GPCRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes” , as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated GPCRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:2n-1, wherein n is an integer between 1-13 or a complement of this aforementioned nucleotide sequence, can be isolated using standard molecular biology techniques and the sequence information provided herein. Using all or a portion of the nucleic acid sequence of SEQ ID NOS:2n-1, wherein n is an integer between 1-13 as a hybridization probe, GPCRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:2n-1, wherein n is an integer between 1-13, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:2n-1, wherein n is an integer between 1-13, or a portion of this nucleotide sequence (e.g., a fragment that can be used as a probe or primer or a fragment encoding a biologically-active portion of an GPCRX polypeptide). A nucleic acid molecule that is complementary to the nucleotide sequence shown in SEQ ID NOS:2n-1, wherein n is an integer between 1-13 is one that is sufficiently complementary to the nucleotide sequence shown in SEQ ID NOS:2n-1, wherein n is an integer between 1-13 that it can hydrogen bond with little or no mismatches to the nucleotide sequence shown SEQ ID NOS:2n-1, wherein n is an integer between 1-13, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be fall length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of GPCRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an GPCRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human GPCRX protein. Homologous nucleic acid sequences include those nucleic acid sequences that encode conservative amino acid substitutions (see below) in SEQ ID NOS:2n-1, wherein n is an integer between 1-13, as well as a polypeptide possessing GPCRX biological activity. Various biological activities of the GPCRX proteins are described below.

An GPCRX polypeptide is encoded by the open reading frame (“ORF”)) of an GPCRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human GPCRX genes allows for the generation of probes and primers designed for use in identifying and/or cloning GPCRX homologues in other cell types, e.g. from other tissues, as well as GPCRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:2n-1, wherein n is an integer between 1-13; or an anti-sense strand nucleotide sequence of SEQ ID NOS:2n-1, wherein n is an integer between 1-13; or of a naturally occurring mutant of SEQ ID NOS:2n-1, wherein n is an integer between 1-13.

Probes based on the human GPCRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an GPCRX protein, such as by measuring a level of an GPCRX-encoding nucleic acid in a sample of cells from a subject e.g., detecting GPCRX mRNA levels or determining whether a genomic GPCRX gene has been mutated or deleted.

“A polypeptide having a biologically-active portion of an GPCRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of GPCRX” can be prepared by isolating a portion SEQ ID NOS:2n-1, wherein n is an integer between 1-13 that encodes a polypeptide having an GPCRX biological activity (the biological activities of the GPCRX proteins are described below), expressing the encoded portion of GPCRX protein (e.g., by recombinant expression in vitro) and assessing the activity of the encoded portion of GPCRX.

### GPCRX Nucleic Acid and Polypeptide Variants

The invention further encompasses nucleic acid molecules that differ from the nucleotide sequences shown SEQ ID NOS:2n-1, wherein n is an integer between 1-13 due to degeneracy of the genetic code and thus encode the same GPCRX proteins as that encoded by the nucleotide sequences shown in SEQ ID NOS:2n-1, wherein n is an integer between 1-13. In another embodiment, an isolated nucleic acid molecule of the invention has a nucleotide sequence encoding a protein having an amino acid sequence shown in SEQ ID NOS:2n, wherein n is an integer between 1-13.

In addition to the human GPCRX nucleotide sequences shown in SEQ ID NOS:2n-1, wherein n is an integer between 1-13 it will be appreciated by those skilled in the art that DNA sequence polymorphisms that lead to changes in the amino acid sequences of the GPCRX polypeptides may exist within a population (e.g., the human population). Such genetic polymorphism in the GPCRX genes may exist among individuals within a population due to natural allelic variation. As used herein, the terms “gene” and “recombinant gene” refer to nucleic acid molecules comprising an open reading frame (ORF) encoding an GPCRX protein, preferably a vertebrate GPCRX protein. Such natural allelic variations can typically result in 1-5% variance in the nucleotide sequence of the GPCRX genes. Any and all such nucleotide variations and resulting amino acid polymorphisms in the GPCRX polypeptides, which are the result of natural allelic variation and that do not alter the functional activity of the GPCRX polypeptides, are intended to be within the scope of the invention.

Moreover, nucleic acid molecules encoding GPCRX proteins from other species, and thus that have a nucleotide sequence that differs from the human sequence SEQ ID NOS:2n-1, wherein n is an integer between 1-13 are intended to be within the scope of the invention. Nucleic acid molecules corresponding to natural allelic variants and homologues of the GPCRX cDNAs of the invention can be isolated based on their homology to the human GPCRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:2n-1, wherein n is an integer between 1-13. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GPCRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS:2n-1, wherein n is an integer between 1-13 corresponds to a naturally-occurring nucleic acid molecule. As used herein, a naturally-occurring“nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:2n-1, wherein n is an integer between 1-13 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:2n-1, wherein n is an integer between 1-13 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

### Conservative Mutations

In addition to naturally-occurring allelic variants of GPCRX sequences that may exist in the population, the skilled artisan will further appreciate that changes can be introduced by mutation into the nucleotide sequences of SEQ ID NOS:2n-1, wherein n is an integer between 1-1 3 thereby leading to changes in the amino acid sequences of the encoded GPCRX proteins, without altering the functional ability of said GPCRX proteins. For example, nucleotide substitutions leading to amino acid substitutions at “non-essential” amino acid residues can be made in the sequence of SEQ ID NOS:2n, wherein n is an integer between 1-13. A “non-essential” amino acid residue is a residue that can be altered from the wild-type sequences of the GPCRX proteins without altering their biological activity, whereas an “essential” amino acid residue is required for such biological activity. For example, amino acid residues that are conserved among the GPCRX proteins of the invention are predicted to be particularly non-amenable to alteration. Amino acids for which conservative substitutions can be made are well-known within the art.

Another aspect of the invention pertains to nucleic acid molecules encoding GPCRX proteins that contain changes in amino acid residues that are not essential for activity. Such GPCRX proteins differ in amino acid sequence from SEQ ID NOS:2n, wherein n is an integer between 1-13 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2n, wherein n is an integer between 1-13. Preferably, the protein encoded by the nucleic acid molecule is at least about 60% homologous to SEQ ID NOS:2n, wherein n is an integer between 1-13; more preferably at least about 70% homologous to SEQ ID NOS:2n, wherein n is an integer between 1-13; still more preferably at least about 80% homologous to SEQ ID NOS:2n, wherein n is an integer between 1-13; even more preferably at least about 90% homologous to SEQ ID NOS:2n, wherein n is an integer between 1-13; and most preferably at least about 95% homologous to SEQ ID NOS:2n, wherein n is an integer between 1-13.

An isolated nucleic acid molecule encoding an GPCRX protein homologous to the protein of SEQ ID NOS:2n, wherein n is an integer between 1-13 can be created by introducing one or more nucleotide substitutions, additions or deletions into the nucleotide sequence of SEQ ID NOS: 2n-1, wherein n is an integer between 1-13 such that one or more amino acid substitutions, additions or deletions are introduced into the encoded protein.

Mutations can be introduced into SEQ ID NOS:2n-1, wherein n is an integer between 1-13 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GPCRX protein is replaced with another amino acid residue from the same side chain family. Alternatively, in another embodiment, mutations can be introduced randomly along all or part of an GPCRX coding sequence, such as by saturation mutagenesis, and the resultant mutants can be screened for GPCRX biological activity to identify mutants that retain activity. Following mutagenesis of SEQ ID NOS:2n-1, wherein n is an integer between 1-13, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant GPCRX protein can be assayed for (i) the ability to form protein:protein interactions with other GPCRX proteins, other cell-surface proteins, or biologically-active portions thereof, (ii) complex formation between a mutant GPCRX protein and an GPCRX ligand; or (iii) the ability of a mutant GPCRX protein to bind to an intracellular target protein or biologically-active portion thereof; (e.g. avidin proteins).

In yet another embodiment, a mutant GPCRX protein can be assayed for the ability to regulate a specific biological function (e.g., regulation of insulin release).

### Antisense Nucleic Acids

Another aspect of the invention pertains to isolated antisense nucleic acid molecules that are hybridizable to or complementary to the nucleic acid molecule comprising the nucleotide sequence of SEQ ID NOS:2n-1, wherein n is an integer between 1-13, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire GPCRX coding strand, or to only a portion thereof. Nucleic acid molecules encoding fragments, homologs, derivatives and analogs of an GPCRX protein of SEQ ID NOS:2n, wherein n is an integer between 1-13, or antisense nucleic acids complementary to an GPCRX nucleic acid sequence of SEQ ID NOS:2n-1, wherein n is an integer between 1-13, are additionally provided.

In one embodiment, an antisense nucleic acid molecule is antisense to a “coding region” of the coding strand of a nucleotide sequence encoding an GPCRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the GPCRX protein. The term “noncoding region” refers to 5′ and 3′ sequences which flank the coding region that are not translated into amino acids (i.e., also referred to as 5′ and 3′ untranslated regions).

Given the coding strand sequences encoding the GPCRX protein disclosed herein, antisense nucleic acids of the invention can be designed according to the rules of Watson and Crick or Hoogsteen base pairing. The antisense nucleic acid molecule can be complementary to the entire coding region of GPCRX mRNA, but more preferably is an oligonucleotide that is antisense to only a portion of the coding or noncoding region of GPCRX mRNA. For example, the antisense ligonucleotide can be complementary to the region surrounding the translation start site of GPCRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an GPCRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an (α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641.

The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

### Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave GPCRX mRNA transcripts to thereby inhibit translation of GPCRX mRNA. A ribozyme having specificity for an GPCRX-encoding nucleic acid can be designed based upon the nucleotide sequence of an GPCRX cDNA disclosed herein (i.e., SEQ ID NOS:2n-1, wherein n is an integer between 1-13). For example, a derivative of a Tetrahymena L-19 IVS RNA can be constructed in which the nucleotide sequence of the active site is complementary to the nucleotide sequence to be cleaved in an GPCRX-encoding mRNA. See, e.g., U.S. Pat. No. 4,987,071 to Cech, et al. and U.S. Pat. No. 5,116,742 to Cech, et al. GPCRX mRNA can also be used to select a catalytic RNA having a specific ribonuclease activity from a pool of RNA molecules. See, e.g., Bartel et al., (1993) Science 261:1411-1418.

Alternatively, GPCRX gene expression can be inhibited by targeting nucleotide sequences complementary to the regulatory region of the GPCRX nucleic acid (e.g., the GPCRX promoter and/or enhancers) to form triple helical structures that prevent transcription of the GPCRX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the GPCRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of GPCRX can be used in therapeutic and diagnostic applications. For example, PNAs can be used as antisense or antigene agents for sequence-specific modulation of gene expression by, e.g., inducing transcription or translation arrest or inhibiting replication. PNAs of GPCRX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of GPCRX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of GPCRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### GPCRX Polypeptides

A polypeptide according to the invention includes a polypeptide including the amino acid sequence of GPCRX polypeptides whose sequences are provided in SEQ ID NOS:2n, wherein n is an integer between 1-13. The invention also includes a mutant or variant protein any of whose residues may be changed from the corresponding residues shown in SEQ ID NOS:2n, wherein n is an integer between 1-13 while still encoding a protein that maintains its GPCRX activities and physiological functions, or a functional fragment thereof.

In general, an GPCRX variant that preserves GPCRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated GPCRX proteins, and biologically-active portions thereof, or derivatives, fragments, analogs or homologs thereof. Also provided are polypeptide fragments suitable for use as immunogens to raise anti-GPCRX antibodies. In one embodiment, native GPCRX proteins can be isolated from cells or tissue sources by an appropriate purification scheme using standard protein purification techniques. In another embodiment, GPCRX proteins are produced by recombinant DNA techniques. Alternative to recombinant expression, an GPCRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the GPCRX protein is derived, or substantially free from chemical precursors or other chemicals when chemically synthesized. The language “substantially free of cellular material” includes preparations of GPCRX proteins in which the protein is separated from cellular components of the cells from which it is isolated or recombinantly-produced. In one embodiment, the language “substantially free of cellular material” includes preparations of GPCRX proteins having less than about 30% (by dry weight) of non-GPCRX proteins (also referred to herein as a “contaminating protein”), more preferably less than about 20% of non-GPCRX proteins, still more preferably less than about 10% of non-GPCRX proteins, and most preferably less than about 5% of non-GPCRX proteins. When the GPCRX protein or biologically-active portion thereof is recombinantly-produced, it is also preferably substantially free of culture medium, i.e., culture medium represents less than about 20%, more preferably less than about 10%, and most preferably less than about 5% of the volume of the GPCRX protein preparation.

The language “substantially free of chemical precursors or other chemicals” includes preparations of GPCRX proteins in which the protein is separated from chemical precursors or other chemicals that are involved in the synthesis of the protein. In one embodiment, the language “substantially free of chemical precursors or other chemicals” includes preparations of GPCRX proteins having less than about 30% (by dry weight) of chemical precursors or non-GPCRX chemicals, more preferably less than about 20% chemical precursors or non-GPCRX chemicals, still more preferably less than about 10% chemical precursors or non-GPCRX chemicals, and most preferably less than about 5% chemical precursors or non-GPCRX chemicals.

Biologically-active portions of GPCRX proteins include peptides comprising amino acid sequences sufficiently homologous to or derived from the amino acid sequences of the GPCRX proteins (e.g., the amino acid sequence shown in SEQ ID NOS:2n, wherein n is an integer between 1-13) that include fewer amino acids than the full-length GPCRX proteins, and exhibit at least one activity of an GPCRX protein. Typically, biologically-active portions comprise a domain or motif with at least one activity of the GPCRX protein. A biologically-active portion of an GPCRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native GPCRX protein.

In an embodiment, the GPCRX protein has an amino acid sequence shown in SEQ ID NOS:2n, wherein n is an integer between 1-13. In other embodiments, the GPCRX protein is substantially homologous to SEQ ID NOS:2n, wherein n is an integer between 1-13, and retains the functional activity of the protein of SEQ ID NOS:2n, wherein n is an integer between 1-13, yet differs in amino acid sequence due to natural allelic variation or mutagenesis, as described in detail, below. Accordingly, in another embodiment, the GPCRX protein is a protein that comprises an amino acid sequence at least about 45% homologous to the amino acid sequence SEQ ID NOS: 2n, wherein n is an integer between 1-13, and retains the functional activity of the GPCRX proteins of SEQ ID NOS:2n, wherein n is an integer between 1-13.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:2n-1, wherein n is an integer between 1-13.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides GPCRX chimeric or fusion proteins. As used herein, an GPCRX “chimeric protein” or “fusion protein” comprises an GPCRX polypeptide operatively-linked to a non-GPCRX polypeptide. An “GPCRX polypeptide” refers to a polypeptide having an amino acid sequence corresponding to an GPCRX protein (SEQ ID NOS:2n, wherein n is an integer between 1-13), whereas a “non-GPCRX polypeptide” refers to a polypeptide having an amino acid sequence corresponding to a protein that is not substantially homologous to the GPCRX protein, e.g., a protein that is different from the GPCRX protein and that is derived from the same or a different organism. Within an GPCRX fusion protein the GPCRX polypeptide can correspond to all or a portion of an GPCRX protein. In one embodiment, an GPCRX fusion protein comprises at least one biologically-active portion of an GPCRX protein. In another embodiment, an GPCRX fusion protein comprises at least two biologically-active portions of an GPCRX protein. In yet another embodiment, an GPCRX fusion protein comprises at least three biologically-active portions of an GPCRX protein. Within the fusion protein, the term “operatively-linked” is intended to indicate that the GPCRX polypeptide and the non-GPCRX polypeptide are fused in-frame with one another. The non-GPCRX polypeptide can be fused to the N-terminus or C-terminus of the GPCRX polypeptide.

In one embodiment, the fusion protein is a GST-GPCRX fusion protein in which the GPCRX sequences are fused to the C-terminus of the GST (glutathione S-transferase) sequences. Such fusion proteins can facilitate the purification of recombinant GPCRX polypeptides.

In another embodiment, the fusion protein is an GPCRX protein containing a heterologous signal sequence at its N-terminus. In certain host cells (e.g., mammalian host cells), expression and/or secretion of GPCRX can be increased through use of a heterologous signal sequence.

In yet another embodiment, the fusion protein is an GPCRX-immunoglobulin fusion protein in which the GPCRX sequences are fused to sequences derived from a member of the immunoglobulin protein family. The GPCRX-immunoglobulin fusion proteins of the invention can be incorporated into pharmaceutical compositions and administered to a subject to inhibit an interaction between an GPCRX ligand and an GPCRX protein on the surface of a cell, to thereby suppress GPCRX-mediated signal transduction in vivo. The GPCRX-immunoglobulin fusion proteins can be used to affect the bioavailability of an GPCRX cognate ligand. Inhibition of the GPCRX ligand/GPCRX interaction may be useful therapeutically for both the treatment of proliferative and differentiative disorders, as well as modulating (e.g. promoting or inhibiting) cell survival. Moreover, the GPCRX-immunoglobulin fusion proteins of the invention can be used as immunogens to produce anti-GPCRX antibodies in a subject, to purify GPCRX ligands, and in screening assays to identify molecules that inhibit the interaction of GPCRX with an GPCRX ligand.

An GPCRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An GPCRX-encoding nucleic acid can be cloned into such an expression vector such that the fusion moiety is linked in-frame to the GPCRX protein.

### GPCRX Agonists and Antagonists

The invention also pertains to variants of the GPCRX proteins that function as either GPCRX agonists (i.e., mimetics) or as GPCRX antagonists. Variants of the GPCRX protein can be generated by mutagenesis (e.g., discrete point mutation or truncation of the GPCRX protein). An agonist of the GPCRX protein can retain substantially the same, or a subset of, the biological activities of the naturally occurring form of the GPCRX protein. An antagonist of the GPCRX protein can inhibit one or more of the activities of the naturally occurring form of the GPCRX protein by, for example, competitively binding to a downstream or upstream member of a cellular signaling cascade which includes the GPCRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCRX proteins.

Variants of the GPCRX proteins that function as either GPCRX agonists (i.e., mimetics) or as GPCRX antagonists can be identified by screening combinatorial libraries of mutants (e.g., truncation mutants) of the GPCRX proteins for GPCRX protein agonist or antagonist activity. In one embodiment, a variegated library of GPCRX variants is generated by combinatorial mutagenesis at the nucleic acid level and is encoded by a variegated gene library. A variegated library of GPCRX variants can be produced by, for example, enzymatically ligating a mixture of synthetic oligonucleotides into gene sequences such that a degenerate set of potential GPCRX sequences is expressible as individual polypeptides, or alternatively, as a set of larger fusion proteins (e.g., for phage display) containing the set of GPCRX sequences therein. There are a variety of methods which can be used to produce libraries of potential GPCRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the GPCRX protein coding sequences can be used to generate a variegated population of GPCRX fragments for screening and subsequent selection of variants of an GPCRX protein. In one embodiment, a library of coding sequence fragments can be generated by treating a double stranded PCR fragment of an GPCRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the GPCRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify GPCRX variants. See, e.g., Arkin and Yourvan, 1992. Proc. Natl. Acad. Sci. USA 89: 7811-7815; Delgrave, et al., 1993. Protein Engineering 6:327-331.

### Anti-GPCRX Antibodies

The invention encompasses antibodies and antibody fragments, such as Fab or (Fab)2, that bind immunospecifically to any of the GPCRX polypeptides of said invention.

An isolated GPCRX protein, or a portion or fragment thereof, can be used as an immunogen to generate antibodies that bind to GPCRX polypeptides using standard techniques for polyclonal and monoclonal antibody preparation. The full-length GPCRX proteins can be used or, alternatively, the invention provides antigenic peptide fragments of GPCRX proteins for use as immunogens. The antigenic GPCRX peptides comprises at least 4 amino acid residues of the amino acid sequence shown in SEQ ID NOS:2n, wherein n is an integer between 1-13 and encompasses an epitope of GPCRX such that an antibody raised against the peptide forms a specific immune complex with GPCRX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of GPCRX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, GPCRX protein sequences of SEQ ID NOS:2n, wherein n is an integer between 1-13, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as GPCRX. Such antibodies include, but are not limited to, polyclonal, monoclonal, chimeric, single chain, F_{ab} and F(_{ab′)2} fragments, and an F_{ab} expression library. In a specific embodiment, antibodies to human GPCRX proteins are disclosed. Various procedures known within the art may be used for the production of polyclonal or monoclonal antibodies to an GPCRX protein sequence of SEQ ID NOS:2n, wherein n is an integer between 1-13, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed GPCRX protein or a chemically-synthesized GPCRX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against GPCRX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of GPCRX. A monoclonal antibody composition thus typically displays a single binding affinity for a particular GPCRX protein with which it immunoreacts. For preparation of monoclonal antibodies directed towards a particular GPCRX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an GPCRX protein (see, e.g., U.S. Pat. No. 4,946,778). In addition, methods can be adapted for the construction of F_{ab} expression libraries (see, e.g., Huse, et al., 1989.

Science 246: 1275-1281) to allow rapid and effective identification of monoclonal F_{ab} fragments with the desired specificity for an GPCRX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an GPCRX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-GPCRX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an GPCRX protein is facilitated by generation of hybridomas that bind to the fragment of an GPCRX protein possessing such a domain. Thus, antibodies that are specific for a desired domain within an GPCRX protein, or derivatives, fragments, analogs or homologs thereof, are also provided herein.

Anti-GPCRX antibodies may be used in methods known within the art relating to the localization and/or quantitation of an GPCRX protein (e.g., for use in measuring levels of the GPCRX protein within appropriate physiological samples, for use in diagnostic methods, for use in imaging the protein, and the like). In a given embodiment, antibodies for GPCRX proteins, or derivatives, fragments, analogs or homologs thereof, that contain the antibody derived binding domain, are utilized as pharmacologically-active compounds (hereinafter “Therapeutics”).

An anti-GPCRX antibody (e.g., monoclonal antibody) can be used to isolate an GPCRX polypeptide by standard techniques, such as affinity chromatography or immunoprecipitation. An anti-GPCRX antibody can facilitate the purification of natural GPCRX polypeptide from cells and of recombinantly-produced GPCRX polypeptide expressed in host cells. Moreover, an anti-GPCRX antibody can be used to detect GPCRX protein (e.g., in a cellular lysate or cell supernatant) in order to evaluate the abundance and pattern of expression of the GPCRX protein. Anti-GPCRX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### GPCRX Recombinant Expression Vectors and Host Cells

Another aspect of the invention pertains to vectors, preferably expression vectors, containing a nucleic acid encoding an GPCRX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleofide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCRX proteins, mutant forms of GPCRX proteins, fusion proteins, etc.).

The recombinant expression vectors of the invention can be designed for expression of GPCRX proteins in prokaryotic or eukaryotic cells. For example, GPCRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the GPCRX expression vector is a yeast expression vector. Examples of vectors for expression in yeast Saccharomyces cerivisae include pYepSecl (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, GPCRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufmnan, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to GPCRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, GPCRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding GPCRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCRX protein. Accordingly, the invention further provides methods for producing GPCRX protein using the host cells of the invention. In one embodiment, the method comprises culturing the host cell of invention (into which a recombinant expression vector encoding GPCRX protein has been introduced) in a suitable medium such that GPCRX protein is produced. In another embodiment, the method further comprises isolating GPCRX protein from the medium or the host cell.

### Transgenic GPCRX Animals

The host cells of the invention can also be used to produce non-human transgenic animals. For example, in one embodiment, a host cell of the invention is a fertilized oocyte or an embryonic stem cell into which GPCRX protein-coding sequences have been introduced. Such host cells can then be used to create non-human transgenic animals in which exogenous GPCRX sequences have been introduced into their genome or homologous recombinant animals in which endogenous GPCRX sequences have been altered. Such animals are useful for studying the function and/or activity of GPCRX protein and for identifying and/or evaluating modulators of GPCRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCRX-encoding nucleic acid into the male pronuclei of a fertilized oocyte (e.g., by microinjection, retroviral infection) and allowing the oocyte to develop in a pseudopregnant female foster animal. The human GPCRX cDNA sequences of SEQ ID NOS:1, 3, 5, 7, 9, 11, 13, 16, 18, 20, 22, 24, 28, 30, 32, 34, 36, 38, and 84 can be introduced as a transgene into the genome of a non-human animal. Alternatively, a non-human homologue of the human GPCRX gene, such as a mouse GPCRX gene, can be isolated based on hybridization to the human GPCRX cDNA (described further supra) and used as a transgene. Intronic sequences and polyadenylation signals can also be included in the transgene to increase the efficiency of expression of the transgene. A tissue-specific regulatory sequence(s) can be operably-linked to the GPCRX transgene to direct expression of GPCRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the GPCRX transgene in its genome and/or expression of GPCRX mRNA in tissues or cells of the animals. A transgenic founder animal can then be used to breed additional animals carrying the transgene. Moreover, transgenic animals carrying a transgene-encoding GPCRX protein can further be bred to other transgenic animals carrying other transgenes.

To create a homologous recombinant animal, a vector is prepared which contains at least a portion of an GPCRX gene into which a deletion, addition or substitution has been introduced to thereby alter, e.g., functionally disrupt, the GPCRX gene. The GPCRX gene can be a human gene (e.g., the cDNA of SEQ ID NOS:1, 3, 5, 7, 9, 11, 13, 16, 18, 20, 22, 24, 28, 30, 32, 34, 36, 38, and 84), but more preferably, is a non-human homologue of a human GPCRX gene. For example, a mouse homologue of human GPCRX gene of SEQ ID NOS:1, 3, 5, 7, 9, 11, 13, 16, 18, 20, 22, 24, 28, 30, 32, 34, 36, 38, and 84 can be used to construct a homologous recombination vector suitable for altering an endogenous GPCRX gene in the mouse genome. In one embodiment, the vector is designed such that, upon homologous recombination, the endogenous GPCRX gene is functionally disrupted (i.e., no longer encodes a functional protein; also referred to as a “knock out” vector).

Alternatively, the vector can be designed such that, upon homologous recombination, the endogenous GPCRX gene is mutated or otherwise altered but still encodes functional protein (e.g., the upstream regulatory region can be altered to thereby alter the expression of the endogenous GPCRX protein). In the homologous recombination vector, the altered portion of the GPCRX gene is flanked at its 5′- and 3′-termini by additional nucleic acid of the GPCRX gene to allow for homologous recombination to occur between the exogenous GPCRX gene carried by the vector and an endogenous GPCRX gene in an embryonic stem cell. The additional flanking GPCRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced GPCRX gene has homologously-recombined with the endogenous GPCRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The GPCRX nucleic acid molecules, GPCRX proteins, and anti-GPCRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an GPCRX protein or anti-GPCRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The isolated nucleic acid molecules of the invention can be used to express GPCRX protein (e.g., via a recombinant expression vector in a host cell in gene therapy applications), to detect GPCRX mRNA (e.g., in a biological sample) or a genetic lesion in an GPCRX gene, and to modulate GPCRX activity, as described further, below. In addition, the GPCRX proteins can be used to screen drugs or compounds that modulate the GPCRX protein activity or expression as well as to treat disorders characterized by insufficient or excessive production of GPCRX protein or production of GPCRX protein forms that have decreased or aberrant activity compared to GPCRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-GPCRX antibodies of the invention can be used to detect and isolate GPCRX proteins and modulate GPCRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

### Screening Assays

The invention provides a method (also referred to herein as a “screening assay”) for identifying modulators, i.e., candidate or test compounds or agents (e.g., peptides, peptidomimetics, small molecules or other drugs) that bind to GPCRX proteins or have a stimulatory or inhibitory effect on, e.g., GPCRX protein expression or GPCRX protein activity. The invention also includes compounds identified in the screening assays described herein.

In one embodiment, the invention provides assays for screening candidate or test compounds which bind to or modulate the activity of the membrane-bound form of an GPCRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of GPCRX protein, or a biologically-active portion thereof, on the cell surface is contacted with a test compound and the ability of the test compound to bind to an GPCRX protein determined. The cell, for example, can of mammalian origin or a yeast cell. Determining the ability of the test compound to bind to the GPCRX protein can be accomplished, for example, by coupling the test compound with a radioisotope or enzymatic label such that binding of the test compound to the GPCRX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of GPCRX protein, or a biologically-active portion thereof, on the cell surface with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX protein or a biologically-active portion thereof as compared to the known compound.

In another embodiment, an assay is a cell-based assay comprising contacting a cell expressing a membrane-bound form of GPCRX protein, or a biologically-active portion thereof, on the cell surface with a test compound and determining the ability of the test compound to modulate (e.g., stimulate or inhibit) the activity of the GPCRX protein or biologically-active portion thereof. Determining the ability of the test compound to modulate the activity of GPCRX or a biologically-active portion thereof can be accomplished, for example, by determining the ability of the GPCRX protein to bind to or interact with an GPCRX target molecule. As used herein, a “target molecule” is a molecule with which an GPCRX protein binds or interacts in nature, for example, a molecule on the surface of a cell which expresses an GPCRX interacting protein, a molecule on the surface of a second cell, a molecule in the extracellular milieu, a molecule associated with the internal surface of a cell membrane or a cytoplasmic molecule. An GPCRX target molecule can be a non-GPCRX molecule or an GPCRX protein or polypeptide of the invention. In one embodiment, an GPCRX target molecule is a component of a signal transduction pathway that facilitates transduction of an extracellular signal (e.g. a signal generated by binding of a compound to a membrane-bound GPCRX molecule) through the cell membrane and into the cell. The target, for example, can be a second intercellular protein that has catalytic activity or a protein that facilitates the association of downstream signaling molecules with GPCRX.

Determining the ability of the GPCRX protein to bind to or interact with an GPCRX target molecule can be accomplished by one of the methods described above for determining direct binding. In one embodiment, determining the ability of the GPCRX protein to bind to or interact with an GPCRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an GPCRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an GPCRX protein or biologically-active portion thereof with a test compound and determining the ability of the test compound to bind to the GPCRX protein or biologically-active portion thereof. Binding of the test compound to the GPCRX protein can be determined either directly or indirectly as described above. In one such embodiment, the assay comprises contacting the GPCRX protein or biologically-active portion thereof with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX or biologically-active portion thereof as compared to the known compound.

In still another embodiment, an assay is a cell-free assay comprising contacting GPCRX protein or biologically-active portion thereof with a test compound and determining the ability of the test compound to modulate (e.g. stimulate or inhibit) the activity of the GPCRX protein or biologically-active portion thereof. Determining the ability of the test compound to modulate the activity of GPCRX can be accomplished, for example, by determining the ability of the GPCRX protein to bind to an GPCRX target molecule by one of the methods described above for determining direct binding. In an alternative embodiment, determining the ability of the test compound to modulate the activity of GPCRX protein can be accomplished by determining the ability of the GPCRX protein further modulate an GPCRX target molecule. For example, the catalytic/enzymatic activity of the target molecule on an appropriate substrate can be determined as described, supra.

In yet another embodiment, the cell-free assay comprises contacting the GPCRX protein or biologically-active portion thereof with a known compound which binds GPCRX protein to form an assay mixture, contacting the assay mixture with a test compound, and determining the ability of the test compound to interact with an GPCRX protein, wherein determining the ability of the test compound to interact with an GPCRX protein comprises determining the ability of the GPCRX protein to preferentially bind to or modulate the activity of an GPCRX target molecule.

The cell-free assays of the invention are amenable to use of both the soluble form or the membrane-bound form of GPCRX protein. In the case of cell-free assays comprising the membrane-bound form of GPCRX protein, it may be desirable to utilize a solubilizing agent such that the membrane-bound form of GPCRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N, N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either GPCRX protein or its target molecule to facilitate separation of complexed from uncomplexed forms of one or both of the proteins, as well as to accommodate automation of the assay. Binding of a test compound to GPCRX protein, or interaction of GPCRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-GPCRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or GPCRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of GPCRX protein binding or activity determined using standard techniques.

Other techniques for immobilizing proteins on matrices can also be used in the screening assays of the invention. For example, either the GPCRX protein or its target molecule can be immobilized utilizing conjugation of biotin and streptavidin. Biotinylated GPCRX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with GPCRX protein or target molecules, but which do not interfere with binding of the GPCRX protein to its target molecule, can be derivatized to the wells of the plate, and unbound target or GPCRX protein trapped in the wells by antibody conjugation. Methods for detecting such complexes, in addition to those described above for the GST-immobilized complexes, include immunodetection of complexes using antibodies reactive with the GPCRX protein or target molecule, as well as enzyme-linked assays that rely on detecting an enzymatic activity associated with the GPCRX protein or target molecule.

In another embodiment, modulators of GPCRX protein expression are identified in a method wherein a cell is contacted with a candidate compound and the expression of GPCRX mRNA or protein in the cell is determined. The level of expression of GPCRX mRNA or protein in the presence of the candidate compound is compared to the level of expression of GPCRX mRNA or protein in the absence of the candidate compound. The candidate compound can then be identified as a modulator of GPCRX mRNA or protein expression based upon this comparison. For example, when expression of GPCRX mRNA or protein is greater (i.e., statistically significantly greater) in the presence of the candidate compound than in its absence, the candidate compound is identified as a stimulator of GPCRX mRNA or protein expression. Alternatively, when expression of GPCRX mRNA or protein is less (statistically significantly less) in the presence of the candidate compound than in its absence, the candidate compound is identified as an inhibitor of GPCRX mRNA or protein expression. The level of GPCRX mRNA or protein expression in the cells can be determined by methods described herein for detecting GPCRX mRNA or protein.

In yet another aspect of the invention, the GPCRX proteins can be used as ” bait proteins” in a two-hybrid assay or three hybrid assay (see, e.g., U.S. Pat. No. 5,283,317; Zervos, et al., 1993. i Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with GPCRX (“GPCRX-binding proteins” or “GPCRX-bp”) and modulate GPCRX activity. Such GPCRX-binding proteins are also likely to be involved in the propagation of signals by the GPCRX proteins as, for example, upstream or downstream elements of the GPCRX pathway.

The two-hybrid system is based on the modular nature of most transcription factors, which consist of separable DNA-binding and activation domains. Briefly, the assay utilizes two different DNA constructs. In one construct, the gene that codes for GPCRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an GPCRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with GPCRX.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments of the GPCRX sequences, SEQ ID NOS:2n 1, wherein n is an integer between 1-13, or fragments or derivatives thereof, can be used to map the location of the GPCRX genes, respectively, on a chromosome. The mapping of the GPCRX sequences to chromosomes is an important first step in correlating these sequences with genes associated with disease.

Briefly, GPCRX genes can be mapped to chromosomes by preparing PCR primers (preferably 15-25 bp in length) from the GPCRX sequences. Computer analysis of the GPCRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the GPCRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCRX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the GPCRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The GPCRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the GPCRX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The GPCRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 16, 18, 20, 22, 24, 28, 30, 32, 34, 36, 38, and 84 are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining GPCRX protein and/or nucleic acid expression as well as GPCRX activity, in the context of a biological sample (e.g., blood, serum, cells, tissue) to thereby determine whether an individual is afflicted with a disease or disorder, or is at risk of developing a disorder, associated with aberrant GPCRX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with GPCRX protein, nucleic acid expression or activity. For example, mutations in an GPCRX gene can be assayed in a biological sample. Such assays can be used for prognostic or predictive purpose to thereby prophylactically treat an individual prior to the onset of a disorder characterized by or associated with GPCRX protein, nucleic acid expression, or biological activity.

Another aspect of the invention provides methods for determining GPCRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of GPCRX in clinical trials.

These and other agents are described in further detail in the following sections.

### Diagnostic Assays

An exemplary method for detecting the presence or absence of GPCRX in a biological sample involves obtaining a biological sample from a test subject and contacting the biological sample with a compound or an agent capable of detecting GPCRX protein or nucleic acid (e.g., mRNA, genomic DNA) that encodes GPCRX protein such that the presence of GPCRX is detected in the biological sample. An agent for detecting GPCRX mRNA or genomic DNA is a labeled nucleic acid probe capable of hybridizing to GPCRX mRNA or genomic DNA. The nucleic acid probe can be, for example, a full-length GPCRX nucleic acid, such as the nucleic acid of SEQ ID NOS:1, 3, 5, 7, 9, 11, 13, 16, 18, 20, 22, 24, 28, 30, 32, 34, 36, 38, and 84, or a portion thereof, such as an oligonucleotide of at least 15, 30, 50, 100, 250 or 500 nucleotides in length and sufficient to specifically hybridize under stringent conditions to GPCRX mRNA or genomic DNA. Other suitable probes for use in the diagnostic assays of the invention are described herein.

An agent for detecting GPCRX protein is an antibody capable of binding to GPCRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect GPCRX mRNA, protein, or genomic DNA in a biological sample in vitro as well as in vivo. For example, in vitro techniques for detection of GPCRX mRNA include Northern hybridizations and in situ hybridizations. In vitro techniques for detection of GPCRX protein include enzyme linked immunosorbent assays (ELISAs), Western blots, immunoprecipitations, and immunofluorescence. In vitro techniques for detection of GPCRX genomic DNA include Southern hybridizations. Furthermore, in vivo techniques for detection of GPCRX protein include introducing into a subject a labeled anti-GPCRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GPCRX protein, mRNA, or genomic DNA, such that the presence of GPCRX protein, mRNA or genomic DNA is detected in the biological sample, and comparing the presence of GPCRX protein, mRNA or genomic DNA in the control sample with the presence of GPCRX protein, mRNA or genomic DNA in the test sample.

The invention also encompasses kits for detecting the presence of GPCRX in a biological sample. For example, the kit can comprise: a labeled compound or agent capable of detecting GPCRX protein or mRNA in a biological sample; means for determining the amount of GPCRX in the sample; and means for comparing the amount of GPCRX in the sample with a standard. The compound or agent can be packaged in a suitable container. The kit can further comprise instructions for using the kit to detect GPCRX protein or nucleic acid.

### Prognostic Assays

The diagnostic methods described herein can furthermore be utilized to identify subjects having or at risk of developing a disease or disorder associated with aberrant GPCRX expression or activity. For example, the assays described herein, such as the preceding diagnostic assays or the following assays, can be utilized to identify a subject having or at risk of developing a disorder associated with GPCRX protein, nucleic acid expression or activity. Alternatively, the prognostic assays can be utilized to identify a subject having or at risk for developing a disease or disorder. Thus, the invention provides a method for identifying a disease or disorder associated with aberrant GPCRX expression or activity in which a test sample is obtained from a subject and GPCRX protein or nucleic acid (e.g., mRNA, genomic DNA) is detected, wherein the presence of GPCRX protein or nucleic acid is diagnostic for a subject having or at risk of developing a disease or disorder associated with aberrant GPCRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GPCRX expression or activity. For example, such methods can be used to determine whether a subject can be effectively treated with an agent for a disorder. Thus, the invention provides methods for determining whether a subject can be effectively treated with an agent for a disorder associated with aberrant GPCRX expression or activity in which a test sample is obtained and GPCRX protein or nucleic acid is detected (e.g., wherein the presence of GPCRX protein or nucleic acid is diagnostic for a subject that can be administered the agent to treat a disorder associated with aberrant GPCRX expression or activity).

The methods of the invention can also be used to detect genetic lesions in an GPCRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an GPCRX-protein, or the misexpression of the GPCRX gene. For example, such genetic lesions can be detected by ascertaining the existence of at least one of: (i) a deletion of one or more nucleotides from an GPCRX gene; (ii) an addition of one or more nucleotides to an GPCRX gene; (iii) a substitution of one or more nucleotides of an GPCRX gene, (iv) a chromosomal rearrangement of an GPCRX gene; (v) an alteration in the level of a messenger RNA transcript of an GPCRX gene, (vi) aberrant modification of an GPCRX gene, such as of the methylation pattern of the genomic DNA, (vii) the presence of a non-wild-type splicing pattern of a messenger RNA transcript of an GPCRX gene, (viii) a non-wild-type level of an GPCRX protein, (ix) allelic loss of an GPCRX gene, and (x) inappropriate post-translational modification of an GPCRX protein. As described herein, there are a large number of assay techniques known in the art which can be used for detecting lesions in an GPCRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GPCRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an GPCRX gene under conditions such that hybridization and amplification of the GPCRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an GPCRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GPCRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GPCRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCRX gene and detect mutations by comparing the sequence of the sample GPCRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GPCRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type GPCRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCRX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an GPCRX sequence, e.g., a wild-type GPCRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GPCRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control GPCRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an GPCRX gene.

Furthermore, any cell type or tissue, preferably peripheral blood leukocytes, in which GPCRX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on GPCRX activity (e.g., GPCRX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of GPCRX protein, expression of GPCRX nucleic acid, or mutation content of GPCRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of GPCRX protein, expression of GPCRX nucleic acid, or mutation content of GPCRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an GPCRX modulator, such as a modulator identified by one of the exemplary screening assays described herein.

### Monitoring of Effects During Clinical Trials

Monitoring the influence of agents (e.g., drugs, compounds) on the expression or activity of GPCRX (e.g., the ability to modulate aberrant cell proliferation and/or differentiation) can be applied not only in basic drug screening, but also in clinical trials. For example, the effectiveness of an agent determined by a screening assay as described herein to increase GPCRX gene expression, protein levels, or upregulate GPCRX activity, can be monitored in clinical trails of subjects exhibiting decreased GPCRX gene expression, protein levels, or downregulated GPCRX activity. Alternatively, the effectiveness of an agent determined by a screening assay to decrease GPCRX gene expression, protein levels, or downregulate GPCRX activity, can be monitored in clinical trails of subjects exhibiting increased GPCRX gene expression, protein levels, or upregulated GPCRX activity. In such clinical trials, the expression or activity of GPCRX and, preferably, other genes that have been implicated in, for example, a cellular proliferation or immune disorder can be used as a “read out” or markers of the immune responsiveness of a particular cell.

By way of example, and not of limitation, genes, including GPCRX, that are modulated in cells by treatment with an agent (e.g., compound, drug or small molecule) that modulates GPCRX activity (e.g., identified in a screening assay as described herein) can be identified. Thus, to study the effect of agents on cellular proliferation disorders, for example, in a clinical trial, cells can be isolated and RNA prepared and analyzed for the levels of expression of GPCRX and other genes implicated in the disorder. The levels of gene expression (i. e., a gene expression pattern) can be quantified by Northern blot analysis or RT-PCR, as described herein, or alternatively by measuring the amount of protein produced, by one of the methods as described herein, or by measuring the levels of activity of GPCRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an GPCRX protein, mRNA, or genomic DNA in the preadministration sample; (iii) obtaining one or more post-administration samples from the subject; (iv) detecting the level of expression or activity of the GPCRX protein, mRNA, or genomic DNA in the post-administration samples; (v) comparing the level of expression or activity of the GPCRX protein, mRNA, or genomic DNA in the pre-administration sample with the GPCRX protein, mRNA, or genomic DNA in the post administration sample or samples; and (vi) altering the administration of the agent to the subject accordingly. For example, increased administration of the agent may be desirable to increase the expression or activity of GPCRX to higher levels than detected, i.e., to increase the effectiveness of the agent. Alternatively, decreased administration of the agent may be desirable to decrease expression or activity of GPCRX to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant GPCRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

### Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant GPCRX expression or activity, by administering to the subject an agent that modulates GPCRX expression or at least one GPCRX activity. Subjects at risk for a disease that is caused or contributed to by aberrant GPCRX expression or activity can be identified by, for example, any or a combination of diagnostic or prognostic assays as described herein. Administration of a prophylactic agent can occur prior to the manifestation of symptoms characteristic of the GPCRX aberrancy, such that a disease or disorder is prevented or, alternatively, delayed in its progression. Depending upon the type of GPCRX aberrancy, for example, an GPCRX agonist or GPCRX antagonist agent can be used for treating the subject. The appropriate agent can be determined based on screening assays described herein. The prophylactic methods of the invention are further discussed in the following subsections.

### Therapeutic Methods

Another aspect of the invention pertains to methods of modulating GPCRX expression or activity for therapeutic purposes. The modulatory method of the invention involves contacting a cell with an agent that modulates one or more of the activities of GPCRX protein activity associated with the cell. An agent that modulates GPCRX protein activity can be an agent as described herein, such as a nucleic acid or a protein, a naturally-occurring cognate ligand of an GPCRX protein, a peptide, an GPCRX peptidomimetic, or other small molecule. In one embodiment, the agent stimulates one or more GPCRX protein activity. Examples of such stimulatory agents include active GPCRX protein and a nucleic acid molecule encoding GPCRX that has been introduced into the cell. In another embodiment, the agent inhibits one or more GPCRX protein activity. Examples of such inhibitory agents include antisense GPCRX nucleic acid molecules and anti-GPCRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an GPCRX protein or nucleic acid molecule. In one embodiment, the method involves administering an agent (e.g., an agent identified by a screening assay described herein), or combination of agents that modulates (e.g., up-regulates or down-regulates) GPCRX expression or activity. In another embodiment, the method involves administering an GPCRX protein or nucleic acid molecule as therapy to compensate for reduced or aberrant GPCRX expression or activity.

Stimulation of GPCRX activity is desirable in situations in which GPCRX is abnormally downregulated and/or in which increased GPCRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The GPCRX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the GPCRX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the GPCRX protein, and the GPCRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

The invention will be further illustrated in the following non-limiting examples.

### EXAMPLE 1

### Identification of GPCRX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### EXAMPLE 2

### Identification of Single Nucleotide Polymorphisms in GPCRX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### EXAMPLE3

### Quantitative Expression Analysis of GPCRX Nucleic Acids in Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by l00. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan® PCR Master Mix for the PE Biosystems 7700, with 5 mM MgC12, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

| | |
|---|---|
| ca. | = carcinoma, |
| * | = established from metastasis, |
| met | = metastasis, |
| s cell var | = small cell variant, |
| non-s | = non-sm = non-small, |
| squam | = squamous, |
| p1. eff | = p1 effusion = pleural effusion, |
| glio | = glioma, |
| astro | = astrocytoma, and |
| neuro | = neuroblastoma. |

### Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

### Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, UT), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 106 cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD10⁶ and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-I beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 107 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

### EXAMPLE 2A

### Expression Analysis of GPCR1 a (ACO11464_A) Nucleic Acid

Expression of gene GPCR1a was assessed using the primer-probe sets Ag1245, Ag1241 and Ag1445, described in Table 11A and 11B. Results of the RTQ-PCR runs are shown in Table 11C, 11D and 11E.

TABLE 11A
Probe Name: Ag1245

| | | | | Start |
|---|---|---|---|---|
| Primers | Sequences | TM | Length | Position |
| Forward | 5′-CCTCTCCGTCGTTTCTTTATTT-3′ | 58.8 | 22 | 757 |
| | (SEQ ID NO:39) | | | |
| Probe | FAM-5′-AGGCATTGGGGTCCACTTCACTTCT-3′-TAMRA | 69 | 25 | 787 |
| | (SEQ ID NO:40) | | | |
| Reverse | 5′-GGAGATTTTCTGGGAAGAGTGA-3′ | 59.7 | 22 | 820 |
| | (SEQ ID NO:41) | | | |

TABLE 11B
Probe Name: Ag1241/Ag1445 (identical sequences)

| | | | | | Start |
|---|---|---|---|---|---|
| Primers | Sequences | | TM | Length | Position |
| Forward | 5′-GAACAAAGCCATCTCCTACATG-3′ | (SEQ ID NO:42) | 58.7 | 22 | 289 |
| Probe | FAM-5′-TGCCTCACACAGGTCTATTTCTCCATG-3′- | (SEQ ID NO:43) | 68.3 | 27 | 314 |
| | TAMRA | | | | |
| Reverse | 5′-TAGCGTGTCCAGAATAGGAAAA-3′ | (SEQ ID NO:44) | 58.9 | 22 | 343 |

TABLE 11C
Panel 1.2

| | Ag1245 | Ag1241 | Ag1241 | Ag1241 |
|---|---|---|---|---|
| | Rel. | Rel. | Rel. | Rel. |
| | Expr., | Expr., | Expr., | Expr., |
| | % | % | % | % |
| | 1.2tm- | 1.2tm- | 1.2tm- | 1.2tm- |
| | 1413- | 1379- | 1481- | 1508- |
| | f_ag- | f_ag- | f_ag- | f_ag- |
| Tissue Name | 1245 | 1241 | 1241 | 1241 |
| Endothelial cells | 0.0 | 0.6 | 0.2 | 0.1 |
| Endothelial cells (treated) | 0.1 | 0.2 | 0.8 | 0.4 |
| Pancreas | 9.8 | 9.2 | 7.7 | 2.5 |
| Pancreatic ca. CAPAN 2 | 0.0 | 0.0 | 0.0 | 0.0 |
| Adrenal Gland (new lot*) | 13.3 | 25.3 | 41.2 | 24.3 |
| Thyroid | 5.7 | 5.1 | 6.3 | 8.4 |
| Salavary gland | 5.2 | 9.0 | 16.8 | 7.9 |
| Pituitary gland | 8.9 | 17.8 | 11.9 | 7.5 |
| Brain (fetal) | 10.5 | 8.4 | 8.5 | 8.0 |
| Brain (whole) | 3.0 | 5.6 | 6.4 | 5.3 |
| Brain (amygdala) | 5.4 | 6.7 | 12.6 | 10.7 |
| Brain (cerebellum) | 5.1 | 9.6 | 10.1 | 3.7 |
| Brain (hippocampus) | 12.1 | 17.2 | 22.7 | 20.9 |
| Brain (thalamus) | 4.2 | 8.8 | 6.8 | 6.5 |
| Cerebral Cortex | 3.8 | 8.1 | 13.9 | 8.7 |
| Spinal cord | 2.1 | 4.1 | 4.7 | 2.5 |
| CNS ca. (glio/astro) U87-MG | 0.1 | 0.0 | 0.8 | 0.6 |
| CNS ca. (glio/astro) U-118-MG | 0.7 | 0.8 | 0.1 | 0.5 |
| CNS ca. (astro) SW1783 | 3.5 | 2.7 | 2.5 | 3.2 |
| CNS ca.* (neuro; met) SK-N-AS | 0.0 | 0.0 | 0.2 | 0.4 |
| CNS ca. (astro) SF-539 | 0.8 | 0.0 | 0.9 | 0.0 |
| CNS ca. (astro) SNB-75 | 0.0 | 0.0 | 0.2 | 0.0 |
| CNS ca. (glio) SNB-19 | 0.8 | 0.6 | 0.7 | 0.1 |
| CNS ca. (glio) U251 | 0.0 | 0.0 | 0.9 | 0.1 |
| CNS ca. (glio) SF-295 | 0.0 | 0.5 | 0.4 | 0.0 |
| Heart | 0.3 | 2.1 | 3.8 | 2.7 |
| Skeletal Muscle (new lot*) | 22.1 | 30.4 | 45.1 | 8.5 |
| Bone marrow | 2.5 | 3.5 | 2.9 | 4.4 |
| Thymus | 2.3 | 3.7 | 5.0 | 11.2 |
| Spleen | 0.9 | 2.4 | 2.6 | 2.2 |
| Lymph node | 2.9 | 7.3 | 9.5 | 3.1 |
| Colorectal | 0.5 | 2.7 | 2.1 | 1.4 |
| Stomach | 3.0 | 6.3 | 8.8 | 4.6 |
| Small intestine | 1.6 | 2.8 | 2.8 | 2.3 |
| Colon ca. SW480 | 0.0 | 0.0 | 0.0 | 0.4 |
| Colon ca.* (SW480 met) SW620 | 0.0 | 0.0 | 0.0 | 0.0 |
| Colon ca. HT29 | 0.5 | 0.8 | 0.2 | 0.1 |
| Colon ca. HCT-116 | 0.7 | 1.5 | 1.6 | 0.2 |
| Colon ca. CaCo-2 | 0.0 | 0.0 | 0.3 | 0.0 |
| 83219 CC Well to Mod Diff | 1.6 | 4.3 | 3.4 | 4.3 |
| (ODO3866) | | | | |
| Colon ca. HCC-2998 | 0.0 | 0.3 | 0.7 | 0.0 |
| Gastric ca.* (liver met) NCI-N87 | 0.2 | 1.4 | 1.6 | 1.3 |
| Bladder | 24.8 | 42.3 | 29.5 | 21.2 |
| Trachea | 2.6 | 4.4 | 7.8 | 9.7 |
| Kidney | 14.2 | 27.4 | 42.3 | 8.6 |
| Kidney (fetal) | 13.2 | 24.3 | 40.1 | 15.8 |
| Renal ca. 786-0 | 0.1 | 0.0 | 0.4 | 0.0 |
| Renal ca. A498 | 0.2 | 0.8 | 0.8 | 0.2 |
| Renal ca. RXF393 | 1.7 | 3.4 | 3.0 | 3.2 |
| Renal ca. ACHN | 0.0 | 0.0 | 0.0 | 0.2 |
| Renal ca. UO-31 | 0.4 | 1.4 | 0.5 | 0.0 |
| Renal Ca. TK-10 | 0.3 | 0.3 | 0.4 | 0.3 |
| Liver | 0.0 | 0.3 | 0.6 | 0.8 |
| Liver (fetal) | 4.0 | 11.7 | 11.0 | 6.7 |
| Liver ca. (hepatoblast) HepG2 | 0.9 | 0.3 | 1.1 | 1.4 |
| Lung | 0.7 | 2.0 | 1.4 | 1.5 |
| Lung (fetal) | 0.9 | 2.7 | 2.2 | 1.8 |
| Lung ca. (small cell) LX-1 | 0.0 | 0.0 | 0.2 | 0.5 |
| Lung ca. (small cell) NCI-H69 | 5.9 | 3.8 | 2.8 | 4.0 |
| Lung ca. (s.cell var.) SHP-77 | 0.0 | 0.2 | 0.5 | 0.0 |
| Lung ca. (large cell) NCI-H460 | 21.9 | 35.4 | 67.4 | 45.4 |
| Lung ca. (non-sm. cell) A549 | 0.6 | 1.6 | 1.1 | 0.3 |
| Lung ca. (non-s.cell) NCI-H23 | 0.0 | 0.2 | 0.0 | 0.0 |
| Lung ca (non-s.cell) HOP-62 | 0.0 | 0.0 | 0.4 | 0.0 |
| Lung ca. (non-s.cl) NCI-H522 | 100.0 | 86.5 | 82.9 | 72.2 |
| Lung ca. (squam.) SW 900 | 27.0 | 56.3 | 54.3 | 25.5 |
| Lung ca. (squam.) NCI-H596 | 2.1 | 2.4 | 2.1 | 0.2 |
| Mammary gland | 0.4 | 0.6 | 2.4 | 1.6 |
| Breast ca.* (p1. effusion) MCF-7 | 0.0 | 0.0 | 0.7 | 0.1 |
| Breast ca.* (p1. ef) MDA-MB-231 | 0.0 | 0.0 | 0.1 | 0.0 |
| Breast ca.* (p1. effusion) T47D | 0.6 | 2.6 | 1.1 | 1.4 |
| Breast ca. BT-549 | 0.6 | 1.2 | 0.9 | 0.0 |
| Breast ca. MDA-N | 0.1 | 0.4 | 0.7 | 1.8 |
| Ovary | 5.4 | 10.4 | 11.0 | 6.3 |
| Ovarian ca. OVCAR-3 | 1.2 | 0.4 | 2.4 | 0.7 |
| Ovarian ca. OVCAR-4 | 0.0 | 0.0 | 0.5 | 0.3 |
| Ovarian ca. OVCAR-5 | 2.4 | 4.6 | 8.1 | 1.9 |
| Ovarian ca. OVCAR-8 | 1.6 | 2.8 | 6.9 | 2.8 |
| Ovarian ca. IGROV-1 | 0.0 | 0.1 | 0.0 | 0.2 |
| Ovarian ca.* (ascites) SK-OV-3 | 0.0 | 0.7 | 0.5 | 0.2 |
| Uterus | 2.7 | 4.2 | 6.2 | 7.6 |
| Plancenta | 5.6 | 12.8 | 13.4 | 6.8 |
| Prostate | 10.4 | 21.2 | 49.3 | 12.7 |
| Prostate ca.* (bone met) PC-3 | 2.0 | 2.7 | 3.3 | 1.6 |
| Testis | 66.9 | 100.0 | 92.7 | 43.2 |
| Melanoma Hs688(A).T | 2.6 | 2.9 | 3.7 | 2.2 |
| Melanoma* (met) Hs688(B).T | 4.9 | 3.2 | 3.0 | 1.4 |
| Melanoma UACC-62 | 0.0 | 0.0 | 0.3 | 0.0 |
| Melanoma M14 | 1.9 | 2.5 | 1.9 | 1.0 |
| Melanoma LOX IMVI | 0.0 | 0.0 | 0.0 | 0.0 |
| Melanoma* (met) SK-MEL-5 | 0.0 | 0.0 | 0.0 | 0.0 |
| Adipose | 21.0 | 78.5 | 100.0 | 100.0 |

TABLE 11D
Panel 4D

| | Ag1245 | Ag1241 | Ag1241 | Ag1241 |
|---|---|---|---|---|
| | Rel. | Rel. | Rel. | Rel. |
| | Expr., | Expr., | Expr., | Expr., |
| | % | % | % | % |
| | 4Dtm- | 4Dtm- | 4Dtm- | 4dtm- |
| | 2105- | 2021- | 2094- | 2474- |
| | f_ag- | f_ag- | f_ag- | f_ag- |
| Tissue Name | 1245 | 1241 | 1241 | 1241 |
| 93768_Secondary | 0.0 | 2.5 | 0.0 | 0.0 |
| Th1_anti-CD28/anti-CD3 | | | | |
| 93769_Secondary | 0.0 | 0.0 | 0.0 | 6.2 |
| Th2_anti-CD28/anti-CD3 | | | | |
| 93770_Secondary | 1.9 | 5.0 | 0.0 | 0.0 |
| Tr1_anti-CD28/anti-CD3 | | | | |
| 93573_Secondary | 0.0 | 1.6 | 2.8 | 0.0 |
| Th1_resting day 4-6 in IL-2 | | | | |
| 93572_Secondary | 0.0 | 2.3 | 0.0 | 2.5 |
| Th2_resting day 4-6 in IL-2 | | | | |
| 93571_Secondary | 0.0 | 0.0 | 0.0 | 1.5 |
| Tr1_resting day 4-6 in IL-2 | | | | |
| 93568_primary | 0.0 | 0.0 | 4.7 | 0.0 |
| Th1_anti-CD28/anti-CD3 | | | | |
| 93569_primary | 2.3 | 4.1 | 2.3 | 0.9 |
| Th2_anti-CD28/anti-CD3 | | | | |
| 93570_primary | 3.9 | 3.9 | 5.3 | 2.6 |
| Tr1_anti-CD28/anti-CD3 | | | | |
| 93565_primary | 43.2 | 92.7 | 92.0 | 95.9 |
| Th1_resting dy 4-6 in IL-2 | | | | |
| 93566_primary | 58.6 | 71.7 | 90.8 | 72.7 |
| Th2_resting dy 4-6 in IL-2 | | | | |
| 93567_primary | 4.1 | 0.0 | 0.0 | 0.0 |
| Tr1_resting dy 4-6 in IL-2 | | | | |
| 93351_CD45RA CD4 | 0.0 | 0.0 | 0.0 | 0.0 |
| lymphocyte_anti-CD28/anti-CD3 | | | | |
| 93352_CD45RO CD4 | 0.0 | 2.5 | 0.0 | 2.1 |
| lymphocyte_anti-CD28/anti-CD3 | | | | |
| 93251_CD8 | 0.0 | 0.0 | 5.0 | 0.0 |
| Lymphocytes_anti-CD28/ | | | | |
| anti-CD3 | | | | |
| 93353_chronic CD8 | 0.0 | 4.5 | 2.7 | 4.6 |
| Lymphocytes 2ry_resting | | | | |
| dy 4-6 in IL-2 | | | | |
| 93574_chronic CD8 | 2.8 | 0.0 | 0.0 | 2.6 |
| Lymphocytes 2ry_activated CD3/ | | | | |
| CD28 | | | | |
| 93354_CD4_none | 0.0 | 0.0 | 0.0 | 0.0 |
| 93252_Secondary | 0.0 | 0.0 | 5.3 | 0.0 |
| Th1/Th2/Tr1_anti-CD95 CH11 | | | | |
| 93103_LAK cells_resting | 36.1 | 66.4 | 70.7 | 25.9 |
| 93788_LAK cells_IL-2 | 1.5 | 14.4 | 15.1 | 2.8 |
| 93787_LAK cells_IL-2 + IL-12 | 14.8 | 13.9 | 27.7 | 2.0 |
| 93789_LAK cells_IL-2 + IFN | 27.2 | 34.9 | 38.2 | 27.7 |
| gamma | | | | |
| 93790_LAK cells_IL-2 + IL-18 | 17.9 | 16.2 | 30.8 | 26.1 |
| 93104_LAK cells_PMA/ | 16.0 | 25.3 | 28.5 | 18.6 |
| ionomycin and IL-18 | | | | |
| 93578_NK Cells IL-2_resting | 0.0 | 2.5 | 2.2 | 0.0 |
| 93109_Mixed Lymphocyte | 30.6 | 23.2 | 32.8 | 27.0 |
| Reaction_Two Way MLR | | | | |
| 93110_Mixed Lymphocyte | 6.9 | 17.1 | 20.0 | 9.9 |
| Reaction_Two Way MLR | | | | |
| 93111_Mixed Lymphocyte | 15.5 | 26.2 | 16.7 | 13.8 |
| Reaction_Two Way MLR | | | | |
| 93112_Mononuclear Cells | 0.0 | 0.0 | 0.0 | 4.9 |
| (PBMCs)_resting | | | | |
| 93113_Mononuclear Cells | 0.0 | 5.8 | 6.2 | 1.9 |
| (PBMCs)_PWM | | | | |
| 93114_Mononuclear Cells | 0.0 | 4.9 | 3.0 | 2.7 |
| (PBMCs)_PHA-L | | | | |
| 93249_Ramos (B cell)_none | 0.0 | 0.0 | 1.1 | 1.2 |
| 93250_Ramos | 0.0 | 0.0 | 2.7 | 1.8 |
| (B cell)_ionomycin | | | | |
| 93349_B lymphocytes_PWM | 0.0 | 1.9 | 8.4 | 3.1 |
| 93350_B lymphoytes_CD40L and | 0.0 | 0.0 | 0.0 | 2.8 |
| IL-4 | | | | |
| 92665_EOL-1 | 0.0 | 2.6 | 0.0 | 0.0 |
| (Eosinophil)_dbcAMP | | | | |
| differentiated | | | | |
| 93248_EOL-1 | 0.0 | 2.6 | 2.8 | 0.0 |
| (Eosinophil)_dbcAMP/ | | | | |
| PMAionomycin | | | | |
| 93356_Dendritic Cells_none | 0.0 | 2.8 | 0.0 | 0.0 |
| 93355_Dendritic Cells_LPS | 0.0 | 0.0 | 0.0 | 0.0 |
| 100 ng/ml | | | | |
| 93775_Dendritic Cells_anti-CD40 | 14.2 | 23.8 | 44.4 | 51.8 |
| 93774_Monocytes_resting | 14.1 | 20.6 | 8.8 | 9.9 |
| 93776_Monocytes_LPS 50 ng/ml | 0.0 | 1.5 | 0.0 | 0.0 |
| 93581_Macrophages_resting | 7.1 | 2.7 | 0.0 | 0.0 |
| 93582_Macrophages_LPS | 0.0 | 0.0 | 2.9 | 0.0 |
| 100 ng/ml | | | | |
| 93098_HUVEC | 0.0 | 0.0 | 2.7 | 0.8 |
| (Endothelial)_none | | | | |
| 93099_HUVEC | 0.0 | 2.2 | 5.8 | 0.0 |
| (Endothelial)_starved | | | | |
| 93100_HUVEC | 3.9 | 0.0 | 3.1 | 0.0 |
| (Endothelial)_IL-1b | | | | |
| 93779_HUVEC | 0.0 | 5.6 | 1.5 | 0.0 |
| (Endothelial)_IFN gamma | | | | |
| 93102_HUVEC | 3.2 | 0.0 | 0.0 | 4.9 |
| (Endothelial)_TNF alpha + IFN | | | | |
| gamma | | | | |
| 93101_HUVEC | 0.0 | 0.0 | 2.4 | 3.1 |
| (Endothelial)_TNF alpha + IL4 | | | | |
| 93781_HUVEC | 0.0 | 0.0 | 0.0 | 0.0 |
| (Endothelial)_IL-11 | | | | |
| 93583_Lung Microvascular | 0.0 | 0.0 | 0.0 | 0.0 |
| Endothelial Cells_none | | | | |
| 93584_Lung Microvascular | 0.0 | 2.0 | 0.0 | 0.0 |
| Endothelial Cells_TNFa (4 ng/ml) | | | | |
| and IL1b (1 ng/ml) | | | | |
| 92662_Microvascular Dermal | 0.0 | 1.6 | 3.3 | 5.3 |
| endothelium_none | | | | |
| 92663_Microsvasular Dermal | 0.0 | 2.5 | 2.1 | 0.0 |
| endothelium_TNFa (4 ng/ml) and | | | | |
| IL1b (1 ng/ml) | | | | |
| 93773_Bronchial epithelium | 52.1 | 31.9 | 87.1 | 28.3 |
| TNFa (4 ng/ml) and IL1b | | | | |
| (1 ng/ml)** | | | | |
| 93347_Small Airway | 3.2 | 0.0 | 2.3 | 0.0 |
| Epithelium_none | | | | |
| 93348_Small Airway | 3.6 | 2.4 | 5.4 | 3.3 |
| Epithelium_TNFa (4 ng/ml) and | | | | |
| IL1b (1 ng/ml) | | | | |
| 92668_Coronery Artery | 9.7 | 5.4 | 8.0 | 21.5 |
| SMC_resting | | | | |
| 92669_Coronery Artery | 6.0 | 9.0 | 15.1 | 14.8 |
| SMC_TNFa (4 ng/ml) and | | | | |
| IL1b (1 ng/ml) | | | | |
| 93107_astrocytes_resting | 0.0 | 2.7 | 0.0 | 0.0 |
| 93108_astrocytes_TNFa | 0.0 | 0.0 | 0.0 | 0.0 |
| (4 ng/ml) and IL1b (1 ng/ml) | | | | |
| 92666_KU-812 | 3.8 | 0.0 | 8.7 | 2.7 |
| (Basophil)_resting | | | | |
| 92667_KU-812 | 0.0 | 0.0 | 9.1 | 2.8 |
| (Basophil)_PMA/ionoycin | | | | |
| 93579_CCD1106 | 0.0 | 2.3 | 0.0 | 0.0 |
| (Keratinocytes)_none | | | | |
| 93580_CCD1106 | 9.4 | 8.1 | 0.0 | 0.0 |
| (Keratinocytes)_TNFa and | | | | |
| IFNg** | | | | |
| 93791_Liver Cirrhosis | 9.7 | 22.1 | 16.7 | 31.0 |
| 93792_Lupus Kidney | 25.9 | 9.3 | 11.0 | 6.5 |
| 93577_NCI-H292 | 0.0 | 0.0 | 0.0 | 0.0 |
| 93358_NCI-H292_IL-4 | 0.0 | 0.0 | 5.6 | 0.0 |
| 93360_NCI-H292_IL-9 | 0.0 | 0.0 | 2.9 | 0.0 |
| 93359_NCI-H292_IL-13 | 0.0 | 0.0 | 0.0 | 0.0 |
| 93357_NCI-H292_IFN gamma | 0.0 | 0.0 | 0.0 | 2.9 |
| 93777_HPAEC_- | 0.0 | 0.0 | 0.0 | 2.0 |
| 93778_HPAEC_IL-1 beta/TNA | 1.4 | 2.2 | 0.0 | 0.0 |
| alpha | | | | |
| 93254_Normal Human Lung | 0.0 | 0.0 | 8.8 | 0.0 |
| Fibroblast_none | | | | |
| 93253_Normal Human Lung | 0.0 | 2.5 | 1.9 | 0.0 |
| Fibroblast_TNFa (4 ng/ml) and | | | | |
| IL-1b (1 ng/ml) | | | | |
| 93257_Normal Human Lung | 0.0 | 0.0 | 0.0 | 0.0 |
| Fibroblast_IL-4 | | | | |
| 93256_Normal Human Lung | 0.0 | 0.0 | 0.0 | 0.0 |
| Fibroblast_IL-9 | | | | |
| 93255_Normal Human Lung | 3.6 | 0.0 | 0.0 | 0.0 |
| Fibroblast_IL-13 | | | | |
| 93258_Normal Human Lung | 0.0 | 0.0 | 0.0 | 0.0 |
| Fibroblast_IFN gamma | | | | |
| 93106_Dermal Fibroblasts | 0.0 | 0.0 | 0.0 | 4.3 |
| CCD1070_resting | | | | |
| 93361_Dermal Fibroblasts | 0.0 | 5.4 | 0.0 | 0.0 |
| CCD1070_TNF alpha 4 ng/ml | | | | |
| 93105_Dermal Fibroblasts | 0.0 | 0.0 | 0.0 | 0.0 |
| CCD1070_IL-1 beta 1 ng/ml | | | | |
| 93772_dermal fibroblast_IFN | 0.0 | 0.0 | 0.0 | 0.0 |
| gamma | | | | |
| 93771_dermal fibroblast_IL-4 | 0.0 | 0.0 | 2.8 | 0.0 |
| 93259_IBD Colitis 1** | 100.0 | 53.6 | 65.5 | 1.1 |
| 93260_IBD Colitis 2 | 0.0 | 0.0 | 0.0 | 0.0 |
| 93261_IBD Crohns | 0.0 | 0.0 | 14.8 | 1.0 |
| 735010_Colon_normal | 4.3 | 0.0 | 0.0 | 0.0 |
| 735019_Lung_none | 4.1 | 2.9 | 4.4 | 2.6 |
| 64028-1_Thymus_none | 66.4 | 100.0 | 100.0 | 58.2 |
| 64030-1_Kidney_none | 26.1 | 85.9 | 92.7 | 100.0 |

Panel 11E. Panels 1.3D and 4D

| | Rel. | | |
|---|---|---|---|
| | Expr., | | |
| | % | | Rel. |
| | 1.3dx- | | Expr., |
| | 4tm- | | % |
| | 5386- | | 4dtm- |
| | f_ag- | | 4759- |
| Panel 1.3D | 1445_- | Panel 4D | f_ag- |
| Tissue Name | a1 | Tissue Name | 1445 |
| Liver | 0.0 | 93768_Secondary | 0.0 |
| adenocarcinoma | | Th1_anti-CD28/anti-CD3 | |
| Pancreas | 14.9 | 93769_Secondary | 0.0 |
| | | Th2_anti-CD28/anti-CD3 | |
| Pancreatic Ca. | 0.0 | 93770_Secondary | 8.1 |
| CAPAN 2 | | Tr1_anti-CD28/anti-CD3 | |
| Adrenal gland | 4.6 | 93573_Secondary | 0.0 |
| | | Th1_resting day 4-6 in IL-2 | |
| Thyroid | 0.0 | 93572_Secondary | 5.8 |
| | | Th2_resting day 4-6 in IL-2 | |
| Salivary gland | 1.3 | 93571_Secondary | 0.0 |
| | | Tr1_resting day 4-6 in IL-2 | |
| Pituitary gland | 0.0 | 93568_primary | 0.0 |
| | | Th1_anti-CD28/anti-CD3 | |
| Brain (fetal) | 23.7 | 93569_primary | 0.0 |
| | | Th2_anti-CD28/anti-CD3 | |
| Brain (whole) | 4.3 | 93570_primary | 3.5 |
| | | Tr1_anti-CD28/anti-CD3 | |
| Brain (amygdala) | 24.7 | 93565_primary | 88.3 |
| | | Th1_resting dy 4-6 in IL-2 | |
| Brain | 11.3 | 93566_primary | 100.0 |
| (cerebellum) | | Th2_resting dy 4-6 in IL-2 | |
| Brain | 12.7 | 93567_primary | 2.9 |
| (hippocampus) | | Tr1_resting dy 4-6 in IL-2 | |
| Brain | 2.0 | 93351_CD45RA CD4 | 2.9 |
| (substantia nigra) | | lymphocyte_anti-CD28/anti-CD3 | |
| Brain (thalamus) | 24.0 | 93352_CD45RO CD4 | 0.0 |
| | | lymphocyte_anti-CD28/anti-CD3 | |
| Cerebral Cortex | 3.4 | 93251_CD8 | 0.0 |
| | | Lymphocytes_anti-CD28/anti- | |
| | | CD3 | |
| Spinal cord | 15.1 | 93353_chronic CD8 Lymphocytes | 0.0 |
| | | 2ry_resting dy 4-6 in IL-2 | |
| CNS ca. (glio/ | 0.0 | 93574_chronic CD8 Lymphocytes | 2.7 |
| astro) U87-MG | | 2ry_activated CD3/CD28 | |
| CNS ca. (glio/ | 0.0 | 93354_CD4_none | 0.0 |
| astro) U-118-MG | | | |
| CNS ca. (astro) | 23.4 | 93252_Secondary | 0.0 |
| SW1783 | | Th1/Th2/Tr1_anti-CD95 CH11 | |
| CNS ca.* (neuro; | 0.0 | 93103_LAK cells_resting | 36.9 |
| met) SK-N-AS | | | |
| CNS ca. (astro) | 0.0 | 93788_LAK cells_IL-2 | 0.0 |
| SF-539 | | | |
| CNS ca. (astro) | 48.7 | 93787_LAK cells_IL-2 + IL-12 | 28.5 |
| SNB-75 | | | |
| CNS ca. (glio) | 5.6 | 93789_LAK cells_IL-2 + IFN | 30.6 |
| SNB-19 | | gamma | |
| CNS ca. (glio) | 0.0 | 93790_LAK cells_IL-2 + IL-18 | 26.2 |
| U251 | | | |
| CNS ca. (glio) | 0.0 | 93104_LAK cells_PMA/ | 34.2 |
| SF-295 | | ionomycin and IL-18 | |
| Heart (fetal) | 0.0 | 93578_NK Cells IL-2_resting | 0.0 |
| Heart | 0.0 | 93109_Mixed Lymphocyte | 36.9 |
| | | Reaction_Two Way MLR | |
| Fetal Skeletal | 7.6 | 93110_Mixed Lymphocyte | 19.6 |
| | | Reaction_Two Way MLR | |
| Skeletal muscle | 0.0 | 93111_Mixed Lymphocyte | 16.3 |
| | | Reaction_Two Way MLR | |
| Bone marrow | 0.0 | 93112_Mononuclear Cells | 0.0 |
| | | (PBMCs)_resting | |
| Thymus | 5.1 | 93113_Mononuclear Cells | 6.4 |
| | | (PBMCs)_PWM | |
| Spleen | 0.0 | 93114_Mononuclear Cells | 0.0 |
| | | (PBMCs)_PHA-L | |
| Lymph node | 23.7 | 93249_Ramos (B cell)_none | 0.0 |
| Colorectal | 2.9 | 93250_Ramos | 8.2 |
| | | (B cell)_ionomycin | |
| Stomach | 5.0 | 93349_B lymphocytes_PWM | 3.3 |
| Small intestine | 0.0 | 93350_B lymphoytes_CD40L | 0.0 |
| | | and IL-4 | |
| Colon ca. SW480 | 0.0 | 92665_EOL-1 | 0.0 |
| | | (Eosinophil)_dbcAMP | |
| | | differentiated | |
| Colon ca.* | 4.4 | 93248_EOL-1 | 2.6 |
| (SW480 met) | | (Eosinophil)_dbcAMP/ | |
| SW620 | | PMAionomycin | |
| Colon ca. HT29 | 0.0 | 93356_Dendritic Cells_none | 0.0 |
| Colon ca. | 0.0 | 93355_Dendritic Cells_LPS | 0.0 |
| HCT-116 | | 100 ng/ml | |
| Colon ca. CaCo-2 | 0.0 | 93775_Dendritic Cells_anti-CD40 | 20.0 |
| 83219 CC Well to | 0.0 | 93774_Monocytes_resting | 17.1 |
| Mod Diff | | | |
| (ODO3866) | | | |
| Colon ca. | 0.0 | 93776_Monocytes_LPS 50 ng/ml | 0.0 |
| HCC-2998 | | | |
| Gastric ca.* | 2.6 | 93581_Macrophages_resting | 0.0 |
| (liver met) | | | |
| NCI-N87 | | | |
| Bladder | 27.6 | 93582_Macrophages_LPS | 0.0 |
| | | 100 ng/ml | |
| Trachea | 0.0 | 93098_HUVEC | 0.0 |
| | | (Endothelial)_none | |
| Kidney | 19.8 | 93099_HUVEC | 0.0 |
| | | (Endothelial)_starved | |
| Kidney (fetal) | 0.0 | 93100_HUVEC | 0.0 |
| | | (Endothelial)_IL-1b | |
| Renal ca. 786-0 | 0.0 | 93779_HUVEC | 0.0 |
| | | (Endothelial)_IFN gamma | |
| Renal ca. A498 | 0.0 | 93102_HUVEC | 3.2 |
| | | (Endothelial)_TNF alpha + IFN | |
| | | gamma | |
| Renal ca. | 3.2 | 93101_HUVEC | 0.0 |
| RXF 393 | | (Endothelial)_TNF alpha + IL4 | |
| Renal ca. ACHN | 0.0 | 93781_HUVEC | 0.0 |
| | | (Endothelial)_IL-11 | |
| Renal ca. UO-31 | 0.0 | 93583_Lung Microvascular | 0.0 |
| | | Endothelial Cells_none | |
| Renal ca. TK-10 | 0.0 | 93584_Lung Microvascular | 0.0 |
| | | Endothelial Cells_TNFa | |
| | | (4 ng/ml) and IL1b (1 ng/ml) | |
| Liver | 0.0 | 92662_Microvascular Dermal | 0.0 |
| | | endothelium_none | |
| Liver (fetal) | 0.0 | 92663_Microsyasular Dermal | 4.5 |
| | | endothelium_TNFa (4 ng/ml) | |
| | | and IL1b (1 ng/ml) | |
| Liver ca. | 4.3 | 93773_Bronchial | 14.2 |
| (hepatoblast) | | epithelium_TNFa (4 ng/ml) and | |
| HepG2 | | IL1b (1 ng/ml)** | |
| Lung | 18.3 | 93347_Small Airway | 0.0 |
| | | Epithelium_none | |
| Lung (fetal) | 4.1 | 93348_Small Airway | 3.8 |
| | | Epithelium_TNFa (4 ng/ml) | |
| | | and IL1b (1 ng/ml) | |
| Lung ca. | 0.0 | 92668_Coronery Artery | 18.9 |
| (small cell) LX-1 | | SMC_resting | |
| Lung ca. | 0.0 | 92669_Coronery Artery | 9.2 |
| (small cell) | | SMC_TNFa (4 ng/ml) and | |
| NCI-H69 | | IL1b (ng/ml) | |
| Lung ca. (s.cell | 4.9 | 93107_astrocytes_resting | 0.0 |
| var.) SHP-77 | | | |
| Lung ca. (large | 21.9 | 93108_astrocytes_TNFa | 3.1 |
| cell) NCI-H460 | | (4 ng/ml) and IL1b (1 ng/ml) | |
| Lung ca. | 0.0 | 92666_KU-812 | 2.7 |
| (non-sm. cell) | | (Basophil)_resting | |
| A549 | | | |
| Lung ca. | 0.0 | 92667_KU-812 | 0.0 |
| (non-s. cell) | | (Basophil)_PMA/ionoycin | |
| NCI-H23 | | | |
| Lung ca | 0.0 | 93579_CCD1106 | 0.0 |
| (non-s. cell) | | (Keratinocytes)_none | |
| HOP-62 | | | |
| Lung ca. | 33.2 | 93580_CCD1106 | 0.0 |
| (non-s. cl) | | (Keratinocytes)_TNFa and | |
| NCI-H522 | | IFNg** | |
| Lung ca. (squam.) | 57.0 | 93791_Liver Cirrhosis | 15.0 |
| SW 900 | | | |
| Lung ca. (squam.) | 0.0 | 93792_Lupus Kidney | 0.0 |
| NCI-H596 | | | |
| Mammary gland | 0.0 | 93577_NCI-H292 | 0.0 |
| Breast ca.* (p1. | 0.0 | 93358_NCI-H292_IL-4 | 0.0 |
| effusion) MCF-7 | | | |
| Breast ca.* | 0.0 | 93360_NCI-H292_IL-9 | 0.0 |
| (p1. ef) | | | |
| MDA-MB-231 | | | |
| Breast ca.* (p1. | 0.0 | 93359_NCI-H292_IL-13 | 0.0 |
| effusion) T47D | | | |
| Breast ca. BT-549 | 4.0 | 93357_NCI-H292_IFN gamma | 0.0 |
| Breast ca. | 0.0 | 93777_HPAEC_- | 3.4 |
| MDA-N | | | |
| Ovary | 15.7 | 93778_HPAE_IL-1 beta/TNA | 0.0 |
| | | alpha | |
| Ovarian ca. | 0.0 | 93254_Normal Human Lung | 2.7 |
| OVCAR-3 | | Fibroblast_none | |
| Ovarian ca. | 0.0 | 93253_Normal Human Lung | 0.0 |
| OVCAR-4 | | Fibroblast_TNFa (4 ng/ml) and | |
| | | IL-1b (1 ng/ml) | |
| Ovarian ca. | 0.0 | 93257_Normal Human Lung | 0.0 |
| OVCAR-5 | | Fibroblast_IL-4 | |
| Ovarian ca. | 4.7 | 93256_Normal Human Lung | 1.9 |
| OVCAR-8 | | Fibroblast_IL-9 | |
| Ovarian ca. | 0.0 | 93255_Normal Human Lung | 6.3 |
| IGROV-1 | | Fibroblast_IL-13 | |
| Ovarian ca.* | 0.0 | 93258_Normal Human Lung | 0.0 |
| (ascites) | | Fibroblast_IFN gamma | |
| SK-OV-3 | | | |
| Uterus | 14.3 | 93106_Dermal Fibroblasts | 0.0 |
| | | CCD1070_resting | |
| Plancenta | 0.0 | 93361_Dermal Fibroblasts | 0.0 |
| | | CCD1070_TNF alpha 4 ng/ml | |
| Prostate | 8.4 | 93105_Dermal Fibroblasts | 0.0 |
| | | CCD1070_IL-1 beta 1 ng/ml | |
| Prostate ca.* | 0.0 | 93772_dermal fibroblast_IFN | 0.0 |
| (bone met) PC-3 | | gamma | |
| Testis | 100.0 | 93771_dermal fibroblast_IL-4 | 0.0 |
| Melanoma | 0.0 | 93259_IBD Colitis 1** | 0.0 |
| Hs688(A).T | | | |
| Melanoma* (met) | 0.0 | 93260_IBD Colitis 2 | 0.0 |
| Hs688(B).T | | | |
| Melanoma | 0.0 | 93261_IBD Crohns | 0.0 |
| UACC-62 | | | |
| Melanoma M14 | 0.0 | 735010_Colon_normal | 9.5 |
| Melanoma | 0.0 | 735019_Lung_none | 2.2 |
| LOX IMVI | | | |
| Melanoma* (met) | 0.0 | 64028-1_Thymus_none | 29.5 |
| SK-MEL-5 | | | |
| Adipose | 0.0 | 64030-1_Kidney_none | 79.6 |

Panel 1.2 Summary: As probed by Ag1245, the GPCR1a gene is largely expressed in normal tissues on panel 1.2 and not in cell lines in this panel. However, it is expressed in 50% (5 of 10) lung cancer cell lines suggesting that the expression of this gene (GPCR1a) may be associated with lung cancer. Thus, therapeutic targeting of this gene may have benefit in lung or other cancers. GPCR1a is also highly expressed in adipose, perhaps reflecting genomic DNA contamination. Highest expression is seen in the testis, with lower expression in brain, prostate, kidney, bladder, adrenal gland and skeletal muscle. Levels are lower still in fetal liver, placenta, uterus, ovary and pancreas. Again, association with many of the lung cancer cell lines is observed.

Panel 1.3 Summary: Expression of GPCR1a with Ag1445 is highest in testis among normal tissues and is seen in many of the lung cancer cell lines, correlating with expression of this gene in panel 1.2.

Panel 4D Summary: Expression of GPCR1a as measured by any of the three probes is consistent. This molecule is highly expressed in resting primary Th1/Th2 cells but not in Tr1 cells or other T cell types. It is also expressed in the thymus and kidney. The receptor encoded for by this transcript is highly expressed in resting Th1/Th2 cells and may be important for the response of these cells to immunomodulatory cytokines and perhaps for the polarization of these cells. Protein therapuetics designed against the protein encoded for by this transcript may enhance or block the differentiation or function of Th1 or Th2 cells. This may be important in the treatment of asthma, allergy, psoriasis, diabetes, arthritis and for organ transplant.

### EXAMPLE 2B

### Expression Analysis of GPCR2a (AC011464_B) Nucleic Acid

Expression of gene GPCR2a was assessed using the primer-probe sets Ag1242 and Ag2024 (identical sequences), described in Table 12A. Results of the RTQ-PCR runs are shown in Table 12B.

TABLE 12A
Probe Name: Ag1242/Ag2024

| | | | | | Start |
|---|---|---|---|---|---|
| Primers | Sequences | | TM | Length | Position |
| Forward | 5′-GTCACCCACTGAGGTACAATGT-3′ | (SEQ ID NO:45) | 58.8 | 22 | 407 |
| Probe | TET-5′-ATCATGAACCCCAAACTCTGTGGGCT-3′- | (SEQ ID NO:46) | 70.4 | 26 | 430 |
| | TAMRA | | | | |
| Reverse | 5′-TAACGATGAAGGACAGCAGAAG-3′ | (SEQ ID NO:47) | 59.5 | 22 | 460 |

TABLE 12B
Panels 1.3D and 1.2

| | Rel. | | Rel. | Rel. |
|---|---|---|---|---|
| | Expr., | | Expr., | Expr., |
| | % | | % | % |
| | 1.3dtm- | | 1.2tm- | 1.2tm- |
| | 3203- | | 1378- | 1447- |
| Panel 1.3D | f_ag- | Panel 1.2 | t_ag- | t_ag- |
| Tissue Name | 2024 | Tissue Name | 1242 | 1242 |
| Liver | 0.0 | Endothelial cells | 0.0 | 0.0 |
| adenocarcinoma | | | | |
| Pancreas | 0.0 | Endothelial cells | 0.0 | 0.0 |
| | | (treated) | | |
| Pancreatic ca. | 0.0 | Pancreas | 0.0 | 0.0 |
| CAPAN 2 | | | | |
| Adrenal gland | 0.0 | Pancreatic ca. | 0.0 | 0.0 |
| | | CAPAN 2 | | |
| Thyroid | 0.0 | Adrenal Gland | 0.0 | 0.0 |
| | | (new lot*) | | |
| Salivary gland | 0.0 | Thyroid | 0.0 | 0.1 |
| Pituitary gland | 0.0 | Salavary gland | 0.0 | 0.0 |
| Brain (fetal) | 0.0 | Pituitary gland | 0.0 | 0.0 |
| Brain (whole) | 12.7 | Brain (fetal) | 0.0 | 0.0 |
| Brain (amygdala) | 0.0 | Brain (whole) | 0.0 | 0.0 |
| Brain (cerebellum) | 0.0 | Brain (amygdala) | 0.0 | 0.1 |
| Brain | 0.0 | Brain (cerebellum) | 0.0 | 0.0 |
| (hippocampus) | | | | |
| Brain | 0.0 | Brain (hippocampus) | 0.0 | 0.0 |
| (substantia nigra) | | | | |
| Brain (thalamus) | 0.0 | Brain (thalamus) | 0.0 | 0.0 |
| Cerebral Cortex | 0.0 | Cerebral Cortex | 0.0 | 0.0 |
| Spinal cord | 0.0 | Spinal cord | 0.8 | 0.0 |
| CNS ca. (glio/astro) | 0.0 | CNS ca. (glio/astro) | 0.0 | 0.0 |
| U87-MG | | U87-MG | | |
| CNS ca. (glio/astro) | 9.3 | CNS ca. (glio/astro) | 0.0 | 0.3 |
| U-118-MG | | U-118-MG | | |
| CNS ca. (astro) | 0.0 | CNS ca. (astro) | 0.0 | 0.0 |
| SW1783 | | SW1783 | | |
| CNS ca.* (neuro; | 0.0 | CNS ca.* (neuro; | 0.0 | 0.6 |
| met) SK-N-AS | | met) SK-N-AS | | |
| CNS ca. (astro) | 0.0 | CNS ca. (astro) | 0.0 | 0.5 |
| SF-539 | | SF-539 | | |
| CNS ca. (astro) | 0.0 | CNS ca. (astro) | 0.0 | 0.5 |
| SNB-75 | | SNB-75 | | |
| CNS ca. (glio) | 0.0 | CNS ca. (glio) | 0.9 | 3.8 |
| SNB-19 | | SNB-19 | | |
| CNS ca. (glio) | 0.0 | CNS ca. (glio) | 0.0 | 0.0 |
| U251 | | U251 | | |
| CNS ca. (glio) | 0.0 | CNS ca. (glio) | 0.0 | 0.0 |
| SF-295 | | SF-295 | | |
| Heart (fetal) | 0.0 | Heart | 0.0 | 0.0 |
| Heart | 0.0 | Skeletal Muscle | 0.0 | 0.0 |
| | | (new lot*) | | |
| Fetal Skeletal | 0.0 | Bone marrow | 0.0 | 0.0 |
| Skeletal muscle | 0.0 | Thymus | 0.0 | 0.0 |
| Bone marrow | 0.0 | Spleen | 0.0 | 0.0 |
| Thymus | 0.0 | Lymph node | 0.0 | 0.0 |
| Spleen | 0.0 | Colorectal | 2.6 | 3.2 |
| Lymph node | 0.0 | Stomach | 0.0 | 0.0 |
| Colorectal | 100.0 | Small intestine | 0.0 | 0.0 |
| Stomach | 0.0 | Colon ca.SW480 | 0.2 | 0.0 |
| Small intestine | 0.0 | Colon ca.* (SW480 | 0.0 | 0.0 |
| | | met) SW620 | | |
| Colon ca. SW480 | 0.0 | Colon ca. HT29 | 0.3 | 0.5 |
| Colon ca.* (SW480 | 0.0 | Colon ca. HCT-116 | 0.0 | 0.0 |
| met) SW620 | | | | |
| Colon ca. HT29 | 0.0 | Colon ca. CaCo-2 | 0.0 | 0.0 |
| Colon ca. | 0.0 | 83219 CC Well to | 7.0 | 7.1 |
| HCT-116 | | Mod Diff (ODO3866) | | |
| Colon ca. CaCo-2 | 0.0 | Colon ca. HCC-2998 | 0.0 | 0.5 |
| 83219 CC Well to | 0.0 | Gastric ca.* | 0.0 | 0.7 |
| Mod Diff | | (livermet) | | |
| (ODO3866) | | NCI-N87 | | |
| Colon ca. HCC-298 | 0.0 | Bladder | 0.0 | 1.4 |
| Gastric ca. (liver | 19.9 | Trachea | 0.0 | 0.0 |
| met) NCI-N87 | | | | |
| Bladder | 0.0 | Kidney | 0.0 | 0.0 |
| Trachea | 0.0 | Kidney (fetal) | 0.0 | 0.0 |
| Kidney | 0.0 | Renal ca. 786-0 | 0.0 | 0.0 |
| Kidney (fetal) | 0.0 | Renal ca. A498 | 0.5 | 0.4 |
| Renal ca. 786-0 | 0.0 | Renal ca. RXF393 | 0.0 | 0.0 |
| Renal ca. A498 | 0.0 | Renal ca. ACHN | 0.0 | 0.0 |
| Renal ca. RXF 393 | 0.0 | Renal ca. UO-31 | 0.9 | 0.0 |
| Renal ca. ACHN | 0.0 | Renal ca. TK-10 | 0.0 | 0.0 |
| Renal ca. UO-31 | 0.0 | Liver | 0.0 | 0.0 |
| Renal ca. TK-10 | 0.0 | Liver (fetal) | 0.0 | 0.0 |
| Liver | 0.0 | Liver ca.(hepatoblast) | 0.0 | 0.0 |
| | | HepG2 | | |
| Liver (fetal) | 0.0 | Lung | 0.0 | 0.0 |
| Liver ca. | 0.0 | Lung (fetal) | 0.0 | 0.0 |
| (hepatoblast) HepG2 | | | | |
| Lung | 0.0 | Lung ca. (small cell) | 0.0 | 0.0 |
| | | LX-1 | | |
| Lung (fetal) | 0.0 | Lung ca. (small cell) | 14.8 | 8.0 |
| | | NCI-H69 | | |
| Lung ca. (small cell) | 0.0 | Lung ca. (s.cell var.) | 0.0 | 0.0 |
| LX- 1 | | SHP-77 | | |
| Lung ca. (small cell) | 0.0 | Lung Ca. (large cell) | 0.0 | 0.2 |
| NCI-H69 | | NCI-H460 | | |
| Lung ca. (s. cell) | 0.0 | Lung ca. (non-sm. | 2.4 | 3.3 |
| var.) SHP-77 | | cell) A549 | | |
| Lung ca. (large | 0.0 | Lung ca. (non-s cell) | 0.0 | 0.0 |
| cell) NCI-H460 | | NCI-H23 | | |
| Lung ca. (non-sm. | 0.0 | Lung ca (non-s.cell) | 0.0 | 0.3 |
| cell) A549 | | HOP-62 | | |
| Lung ca. (non-s. | 0.0 | Lung ca. (non-s.d) | 0.0 | 0.0 |
| cell) NCI-H23 | | NCI-H522 | | |
| Lung ca (non-s. | 0.0 | Lung ca. (squam.) | 0.0 | 0.0 |
| cell) HOP-62 | | SW 900 | | |
| Lung ca. (non-s.d) | 0.0 | Lung ca. (squam.) | 1.5 | 3.9 |
| NCI-H522 | | NCI-H596 | | |
| Lung ca. (squam.) | 0.0 | Mammary gland | 0.0 | 0.0 |
| SW 900 | | | | |
| Lung ca. (squam.) | 0.0 | Breast ca.* (p1. | 0.0 | 0.0 |
| NCI-H596 | | effusion) MCF-7 | | |
| Mammary gland | 0.0 | Breast ca.* (p1.ef) | 0.0 | 0.0 |
| | | MDA-MB-231 | | |
| Breast ca.* (p1. | 0.0 | Breast ca.* (p1. | 0.0 | 1.4 |
| effusion) MCF-7 | | effusion) T47D | | |
| Breast ca.* (p1.ef) | 0.0 | Breast ca. | 0.0 | 0.6 |
| MDA-MB-231 | | BT-549 | | |
| Breast ca.* (p1. | 0.0 | Breast Ca. | 0.0 | 1.4 |
| effusion) T47D | | MDA-N | | |
| Breast ca. BT-549 | 9.0 | Ovary | 0.0 | 0.0 |
| Breast ca. | 0.0 | Ovarian ca. | 8.9 | 3.7 |
| MDA-N | | OVCAR-3 | | |
| Ovary | 0.0 | Ovarian ca. | 0.0 | 0.0 |
| | | OVCAR-4 | | |
| Ovarian ca. | 37.6 | Ovarian ca. | 5.7 | 9.9 |
| OVCAR-3 | | OVCAR-5 | | |
| Ovarian ca. | 0.0 | Ovarian ca. | 0.0 | 2.3 |
| OVCAR-4 | | OVCAR-8 | | |
| Ovarian ca. | 0.0 | Ovarian ca. | 0.0 | 0.6 |
| OVCAR-5 | | IGROV-1 | | |
| Ovarian ca. | 0.0 | Ovarian ca.* (ascites) | 0.0 | 0.1 |
| OVCAR-8 | | SK-OV-3 | | |
| Ovarian ca. | 0.0 | Uterus | 0.0 | 0.0 |
| IGROV-1 | | | | |
| Ovarian ca.* | 0.0 | Plancenta | 0.0 | 0.0 |
| (ascites) SK-OV-3 | | | | |
| Uterus | 22.1 | Prostate | 0.0 | 0.0 |
| Plancenta | 0.0 | Prostate ca.* | 0.0 | 1.0 |
| | | (bone met) PC-3 | | |
| Prostate | 0.0 | Testis | 0.7 | 0.0 |
| Prostate ca.* | 0.0 | Melanoma | 0.0 | 0.0 |
| (bone met) PC-3 | | Hs688(A).T | | |
| Testis | 25.9 | Melanoma* (met) | 1.2 | 1.3 |
| | | Hs688(B).T | | |
| Melanoma | 0.0 | Melanoma | 0.0 | 0.0 |
| Hs688(A).T | | UACC-62 | | |
| Melanoma* | 0.0 | Melanoma | 2.0 | 2.7 |
| (met) Hs688(B).T | | M14 | | |
| Melanoma | 0.0 | Melanoma | 0.0 | 0.0 |
| UACC-62 | | LOX IMVI | | |
| Melanoma | 0.0 | Melanoma* (met) | 0.0 | 0.0 |
| M14 | | SK-MEL-5 | | |
| Melanoma | 0.0 | Adipose | 100.0 | 100.0 |
| LOX IMVI | | | | |
| Melanoma* | 0.0 | | | |
| (met) SK-MEL-5 | | | | |
| Adipose | 0.0 | | | |

Panel 1.2 Summary: GPCR2 is most highly expressed in the adipose sample. This sample is known to be contaminated with genomic DNA, thus the results are not accountable. Not including the sample of adipose results in a predominant expression displayed in cell lines rather than normal tissues. The only non-cell line sample is colon. Thus, this gene may be associated with cells undergoing cell division (a common characteristic of cell lines (and colonic tissue) versus most other tissues) or associated with cancer as these cell lines are all derived from cancers.

Thus, therapeutic targeting of this gene may be useful to treat cancers or other diseases associated with cellular proliferation.

Panel 1.3D Summary: Gene GPCR2 is expressed only in colorectal tissue which correlates with expression seen in panel 1.2. However, expression in cell lines is not seen. Panel 4D Summary: This gene is expressed at low/undetectable levels in panel 4D (data not shown).

### EXAMPLE 3C

### Expression Analysis of GPCR3a (GM39728201_A) Nucleic Acid

Expression of gene GPCR3a was assessed using the primer-probe set Ag1243, described in Table 13A. Results of the RTQ-PCR runs are shown in Table 13B.

TABLE 13A
Probe Name: Ag1243

| | | | | | Start |
|---|---|---|---|---|---|
| Primers | Sequences | | TM | Length | Position |
| Forward | 5′-ATCCAACTCACCTGTTCAGACA-3′ | (SEQ ID NO:48) | 59.6 | 22 | 623 |
| Probe | FAM-5′-CATCCTGATATATTTTGCAGCTTGCA-3′- | (SEQ ID NO:49) | 65.2 | 26 | 658 |
| | TAMRA | | | | |
| Reverse | 5′-GACAGAGGAACACCACCAAATA-3′ | (SEQ ID NO:50) | 59 | 22 | 684 |

TABLE 13B
Panel 1.2

| | Rel. | | Rel. |
|---|---|---|---|
| | Expr., % | | Expr., % |
| | 1.2tm1412f_ag | Tissue | 1.2tm1412f_ag |
| Tissue Name | 1243 | Name | 1243 |
| Endothelial | 0.0 | Renal ca. 786-0 | 0.0 |
| cells | | | |
| Endothelial | 0.0 | Renal ca. A498 | 0.0 |
| cells | | | |
| (treated) | | | |
| Pancreas | 0.0 | Renal ca. RXF 393 | 0.0 |
| Pancreatic | 0.0 | Renal ca. ACHN | 0.0 |
| ca. | | | |
| CAPAN 2 | | | |
| Adrenal | 0.0 | Renal ca. UO-31 | 0.0 |
| Gland | | | |
| (new lot*) | | | |
| Thyroid | 0.0 | Renal ca. TK-10 | 0.0 |
| Salavary | 0.0 | Liver | 0.0 |
| gland | | | |
| Pituitary | 0.0 | Liver | 0.0 |
| gland | | (fetal) | |
| Brain | 0.0 | Liver ca. (hepatoblast) | 0.0 |
| (fetal) | | HepG2 | |
| Brain | 0.0 | Lung | 0.0 |
| (whole) | | | |
| Brain | 0.0 | Lung (fetal) | 0.0 |
| (amygdala) | | | |
| Brain | 0.0 | Lung ca. (small cell) | 0.0 |
| (cerebellum) | | LX-1 | |
| Brain | 0.0 | Lung ca. (small cell) | 1.4 |
| (hippo- | | NCI-H69 | |
| campus) | | | |
| Brain | 0.0 | Lung ca. (s. cell var.) | 0.0 |
| (thalamus) | | SHP-77 | |
| Cerebral | 0.0 | Lung ca. (large cell) | 0.0 |
| Cortex | | NCI-H460 | |
| Spinal cord | 0.0 | Lung ca. (non-sm. cell) | 0.0 |
| | | A549 | |
| CNS ca. | 0.0 | Lung ca. (non-s. cell) | 0.0 |
| (glio/astro) | | NCI-H23 | |
| U87-MG | | | |
| CNS ca. | 0.0 | Lung ca (non-s. cell) | 0.0 |
| (glio/astro) | | HOP-62 | |
| U-118-MG | | | |
| CNS ca. | 0.0 | Lung ca. (non-s. cl) | 0.0 |
| (astro) | | NCI-H522 | |
| SW1783 | | | |
| CNS ca.* | 0.0 | Lung ca. (squam.) | 0.0 |
| (neuro; | | SW 900 | |
| met) | | | |
| SK-N-AS | | | |
| CNS ca. | 0.0 | Lung ca. (squam.) | 0.0 |
| (astro) | | NCI-H596 | |
| SF-539 | | | |
| CNS ca. | 0.0 | Mammary | 0.0 |
| (astro) | | gland | |
| SNB-75 | | | |
| CNS ca. | 0.0 | Breast ca.* | 0.0 |
| (glio) | | (pl. effusion) | |
| SNB-19 | | MCF-7 | |
| CNS ca. | 0.0 | Breast ca.* | 0.0 |
| (glio) | | (pl. ef) | |
| U251 | | MDA- | |
| | | MB-231 | |
| CNS ca. | 0.0 | Breast ca.* | 0.0 |
| (glio) | | (pl. | |
| SF-295 | | effusion) | |
| | | T47D | |
| Heart | 0.0 | Breast ca. BT-549 | 0.0 |
| Skeletal | 0.0 | Breast ca. MDA-N | 0.0 |
| Muscle | | | |
| (new lot*) | | | |
| Bone | 0.0 | Ovary | 0.0 |
| marrow | | | |
| Thymus | 0.0 | Ovarian ca. OVCAR-3 | 18.7 |
| Spleen | 0.0 | Ovarian ca. OVCAR-4 | 0.0 |
| Lymph node | 0.0 | Ovarian ca. OVCAR-5 | 0.0 |
| Colorectal | 0.0 | Ovarian ca. OVCAR-8 | 0.0 |
| Stomach | 0.0 | Ovarian ca. IGROV-1 | 0.0 |
| Small | 0.0 | Ovarian ca.* (ascites) | 0.0 |
| intestine | | SK-OV-3 | |
| Colon ca. | 0.0 | Uterus | 0.0 |
| SW480 | | | |
| Colon ca.* | 0.0 | Plancenta | 0.0 |
| (SW480 | | | |
| met) | | | |
| SW620 | | | |
| Colon ca. | 0.0 | Prostate | 0.0 |
| HT29 | | | |
| Colon ca. | 0.0 | Prostate ca.* | 0.0 |
| HCT-116 | | (bone met) | |
| | | PC-3 | |
| Colon ca. | 0.0 | Testis | 0.0 |
| CaCo-2 | | | |
| 83219 CC | 1.8 | Melanoma Hs688 (A).T | 0.0 |
| Well to | | | |
| Mod Diff | | | |
| (ODO3866) | | | |
| Colon ca. | 0.0 | Melanoma* (met) Hs688 | 0.0 |
| HCC-2998 | | (B).T | |
| Gastric ca.* | 0.0 | Melanoma UACC-62 | 0.0 |
| (liver met) | | | |
| NCI-N87 | | | |
| Bladder | 0.0 | Melanoma M14 | 0.0 |
| Trachea | 0.0 | Melanoma LOX IMVI | 0.0 |
| Kidney | 0.0 | Melanoma* (met) | 0.0 |
| | | SK-MEL-5 | |
| Kidney | 0.0 | Adipose | 100.0 |
| (fetal) | | | |

Panel 1.2 Summary: Expression of gene GPRC3a is skewed by level of expression in the adipose, which is known to be contaminated with genomic DNA. Taking that into consideration, the only sample showing expression is an ovarian cancer cell line. Therefore therapeutics targeted to this GPCR may be effective in particular kinds of cancer.

Panel 4D Summary: Expression is low/undetectable in this panel (data not shown).

### EXAMPLE 3D

### Expression Analysis of GPCR4 (AC011464_D) Nucleic Acid

Expression of gene GPCR4 was assessed using the primer-probe set Agl 244, described in Table 14A. Results of the RTQ-PCR runs are shown in Table 14B.

TABLE 14A
Probe Name: Ag1244

| | | | | | Start |
|---|---|---|---|---|---|
| Primers | Sequences | | TM | Length | Position |
| Forward | 5′-TGTTCTTCTGTGAAGTCGTTCA-3′ | (SEQ ID NO:51) | 58.5 | 22 | 591 |
| Probe | TET-5′-TGACACCCTCATCAACAACATCCTCA-3′- | (SEQ ID NO:52) | 68.9 | 26 | 634 |
| | TAMRA | | | | |
| Reverse | 5′-TGCACCAAATACACTACTTGCA-3′ | (SEQ ID NO:53) | 59.3 | 22 | 667 |

TABLE 14B
Panel 1.2

| | Rel. Expr., % | Rel. Expr., % |
|---|---|---|
| Tissue Name | 1.2tm1379t_ag1244 | 1.2tm1488t_ag1244 |
| Endothelial cells | 0.0 | 0.0 |
| Endothelial cells (treated) | 0.0 | 0.0 |
| Pancreas | 0.0 | 0.0 |
| Pancreatic ca. CAPAN 2 | 0.0 | 0.0 |
| Adrenal Gland (new lot*) | 0.0 | 0.2 |
| Thyroid | 0.0 | 0.0 |
| Salavary gland | 0.0 | 0.0 |
| Pituitary gland | 0.0 | 0.0 |
| Brain (fetal) | 0.0 | 0.0 |
| Brain (whole) | 0.0 | 0.0 |
| Brain (amygdala) | 0.0 | 0.0 |
| Brain (cerebellum) | 0.0 | 0.7 |
| Brain (hippocampus) | 0.0 | 0.0 |
| Brain (thalamus) | 0.0 | 0.0 |
| Cerebral Cortex | 0.0 | 0.0 |
| Spinal cord | 0.0 | 0.0 |
| CNS ca. (glio/astro) U87-MG | 0.0 | 0.0 |
| CNS ca. (glio/astro) | 0.6 | 0.5 |
| U-118-MG | | |
| CNS ca. (astro) SW1783 | 0.0 | 0.2 |
| CNS ca.* (neuro; met) | 0.0 | 0.2 |
| SK-N-AS | | |
| CNS ca. (astro) SF-539 | 0.0 | 0.2 |
| CNS ca. (astro) SNB-75 | 0.0 | 0.0 |
| CNS ca. (glio) SNB-19 | 0.7 | 0.6 |
| CNS ca. (glio) U251 | 0.0 | 0.2 |
| CNS ca. (glio) SF-295 | 0.0 | 0.2 |
| Heart | 0.0 | 0.0 |
| Skeletal Muscle (new lot*) | 0.0 | 0.0 |
| Bone marrow | 0.0 | 0.0 |
| Thymus | 0.0 | 0.0 |
| Spleen | 0.0 | 0.0 |
| Lymph node | 0.0 | 0.3 |
| Colorectal | 0.1 | 0.7 |
| Stomach | 0.0 | 0.0 |
| Small intestine | 0.0 | 0.0 |
| Colon ca. SW480 | 0.0 | 0.0 |
| Colon ca.* (SW480 met) | 0.0 | 0.0 |
| SW620 | | |
| Colon ca. HT29 | 0.0 | 0.3 |
| Colon ca. HCT-116 | 0.0 | 0.1 |
| Colon ca. CaCo-2 | 0.0 | 0.0 |
| 83219 CC Well to Mod Diff | 4.0 | 4.0 |
| (ODO3866) | | |
| Colon ca. HCC-2998 | 0.0 | 0.2 |
| Gastric ca.* (liver met) | 0.0 | 0.1 |
| NCI-N87 | | |
| Bladder | 0.0 | 0.5 |
| Trachea | 0.4 | 0.4 |
| Kidney | 0.0 | 0.8 |
| Kidney (fetal) | 0.0 | 1.0 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) | 4.4 | 4.4 |
| NCI-H69 | | |
| Lung ca. (s. cell var.) SHP-77 | 0.0 | 0.0 |
| Lung ca. (large cell) | 0.0 | 0.3 |
| NCI-H460 | | |
| Lung ca. (non-sm. cell) A549 | 0.6 | 0.5 |
| Lung ca. (non-s. cell) | 0.0 | 0.0 |
| NCI-H23 | | |
| Lung ca (non-s. cell) HOP-62 | 0.0 | 0.3 |
| Lung ca. (non-s. cl) | 0.0 | 0.0 |
| NCI-H522 | | |
| Lung ca. (squam.) SW 900 | 0.0 | 0.0 |
| Lung ca. (squam.) NCI-H596 | 0.0 | 0.2 |
| Mammary gland | 0.0 | 0.0 |
| Breast ca.* | 0.0 | 0.0 |
| (pl. effusion) MCF-7 | | |
| Breast ca.* | 0.0 | 0.0 |
| (pl. ef) MDA-MB-231 | | |
| Breast ca.* (pl. effusion) | 0.4 | 1.0 |
| T47D | | |
| Breast ca. BT-549 | 0.0 | 1.0 |
| Breast ca. MDA-N | 0.0 | 0.7 |
| Ovary | 0.0 | 0.0 |
| Ovarian ca. OVCAR-8 | 2.8 | 0.9 |
| Ovarian ca. OVCAR-4 | 0.0 | 0.0 |
| Ovarian ca. OVCAR-5 | 4.2 | 1.7 |
| Ovarian ca. OVCAR-8 | 0.2 | 0.6 |
| Ovarian ca. IGROV-1 | 0.6 | 0.2 |
| Ovarian ca.* | 0.0 | 0.0 |
| (ascites) SK-OV-3 | | |
| Uterus | 0.0 | 0.0 |
| Plancenta | 0.0 | 0.0 |
| Prostate | 0.0 | 0.0 |
| Prostate ca.* (bone met) PC-3 | 0.0 | 0.4 |
| Testis | 0.0 | 0.3 |
| Melanoma Hs688(A).T | 0.0 | 0.0 |
| Melanoma* (met) Hs688(B).T | 0.0 | 0.6 |
| Melanoma UACC-62 | 0.0 | 0.0 |
| Melanoma M14 | 1.0 | 1.4 |
| Melanoma LOX IMVI | 0.0 | 0.0 |
| Melanoma* (met) SK-MEL-5 | 0.0 | 0.0 |
| Adipose | 100.0 | 100.0 |

Panel 1.2 Summary: Expression of GPCR4 is skewed by expression in adipose, which is known to be due to genomic DNA contamination. Taking that into account, expression is only seen in a lung cancer cell line and a colon cancer sample. This indicates that therapeutics designed to this receptor may be effective therapies in these diseases.

### EXAMPLE 3E

### Expression Analysis of GPCR8b (CG54743-02) Nucleic Acid

Expression of gene GPCR8b was assessed using the primer-probe set Ag1248, described in Table 15A. Results of the RTQ-PCR runs are shown in Table 15B.

TABLE 15A
Probe Name: Ag1248

| | | | | | Start |
|---|---|---|---|---|---|
| Primers | Sequences | | TM | Length | Position |
| Forward | 5′-TTGGCTACTTTTGCTTCACTTC-3′ | (SEQ ID NO:54) | 58.7 | 22 | 991 |
| Probe | TET-5′-TTCTTCTATGGATGTCTCAACCGGCA-3′- | (SEQ ID NO:55) | 68.7 | 26 | 1020 |
| | TAMRA | | | | |
| Reverse | 5′-GCTTGAAGAAGCAGACAAACTG-3′ | (SEQ ID NO:56) | 59.3 | 22 | 1068 |

TABLE 15B
Panels 1.2 and 4D

| | Rel. Expr., % | | Rel. Expr., % | Rel. Expr., % |
|---|---|---|---|---|
| Panel 4D | 4Dtm2107t_{—} | Panel 1.2 | 1.2tm1380t_{—} | 1.2tm1408t_{—} |
| Tissue Name | ag1248 | Tissue Name | ag1248 | ag1248 |
| 93768_Secondary Th1_anti- | 11.0 | Endothelial cells | 1.2 | 0.0 |
| CD28/anti-CD3 | | | | |
| 93769_Secondary Th2_anti- | 12.7 | Endothelial cells (treated) | 2.6 | 2.9 |
| CD28/anti-CD3 | | | | |
| 93770_Secondary Tr1_anti- | 0.0 | Pancreas | 0.9 | 1.2 |
| CD28/anti-CD3 | | | | |
| 93573_Secondary Th1_resting day | 6.2 | Pancreatic ca. CAPAN 2 | 4.8 | 0.0 |
| 4-6 in IL-2 | | | | |
| 93572_Secondary Th2_resting day | 35.6 | Adrenal Gland (new lot*) | 3.7 | 3.4 |
| 4-6 in IL-2 | | | | |
| 93571_Secondary Tr1_resting day | 16.6 | Thyroid | 4.2 | 5.2 |
| 4-6 in IL-2 | | | | |
| 93568 primary Th1_anti- | 0.0 | Salavary gland | 2.7 | 0.5 |
| CD28/anti-CD3 | | | | |
| 93569_primary Th2_anti- | 0.0 | Pituitary gland | 31.9 | 50.3 |
| CD28/anti-CD3 | | | | |
| 93570_primary Trl_anti- | 0.0 | Brain (fetal) | 15.8 | 21.3 |
| CD28/anti-CD3 | | | | |
| 93565_primary Th1_resting dy 4-6 | 25.2 | Brain (whole) | 53.6 | 63.3 |
| in IL-2 | | | | |
| 93566_primary Th2_resting dy 4-6 | 5.9 | Brain (amygdala) | 23.7 | 19.5 |
| in IL-2 | | | | |
| 93567_primary Tr1_resting dy 4-6 | 0.0 | Brain (cerebellum) | 27.7 | 51.4 |
| in IL-2 | | | | |
| 93351_CD45RA CD4 | 6.6 | Brain (hippocampus) | 40.6 | 53.6 |
| lymphocyte_anti-CD28/anti-CD3 | | | | |
| 93352_CD45RO CD4 | 17.7 | Brain (thalamus) | 26.2 | 27.2 |
| lymphocyte_anti-CD28/anti-CD3 | | | | |
| 93251_CD8 Lymphocytes_anti- | 28.3 | Cerebral Cortex | 100.0 | 100.0 |
| CD28/anti-CD3 | | | | |
| 93353_chronic CD8 Lymphocytes | 20.2 | Spinal cord | 3.8 | 3.7 |
| 2ry_resting dy 4-6 in IL-2 | | | | |
| 93574_chronic CD8 Lymphocytes | 0.0 | CNS ca. (glio/astro) U87-MG | 2.6 | 0.1 |
| 2ry_activated CD3/CD28 | | | | |
| 93354_CD4_none | 26.1 | CNS ca. (glio/astro) U-118-MG | 3.0 | 0.6 |
| 93252_Secondary | 0.0 | CNS ca. (astro) SW1783 | 2.5 | 0.8 |
| Th1/Th2/Tr1_anti-CD95 CH11 | | | | |
| 93103_LAK cells_resting | 0.0 | CNS ca.* (neuro; met) SK-N-AS | 8.0 | 1.1 |
| 93788_LAK cells_IL-2 | 25.7 | CNS ca. (astro) SF-539 | 4.1 | 1.3 |
| 93787_LAK cells_IL-2 + IL-12 | 6.1 | CNS ca. (astro) SNB-75 | 2.2 | 1.1 |
| 93789_LAK cells_IL-2 + IFN gamma | 8.5 | CNS ca. (glio) SNB-19 | 4.9 | 3.3 |
| 93790_LAK cells_IL-2 + IL-18 | 6.9 | CNS ca. (glio) U251 | 1.5 | 0.6 |
| 93104_LAK | 10.5 | CNS ca. (glio) SF-295 | 1.4 | 1.3 |
| cells_PMA/ionomycin and IL-18 | | | | |
| 93578_NK Cells IL-2_resting | 23.8 | Heart | 3.3 | 3.7 |
| 93109_Mixed Lymphocyte | 46.7 | Skeletal Muscle (new lot*) | 8.1 | 12.5 |
| Reaction_Two Way MLR | | | | |
| 93110_Mixed Lymphocyte | 9.2 | Bone marrow | 0.3 | 0.0 |
| Reaction_Two Way MLR | | | | |
| 93111_Mixed Lymphocyte | 12.5 | Thymus | 2.8 | 0.7 |
| Reaction_Two Way MLR | | | | |
| 93112_Mononuclear Cells | 11.3 | Spleen | 0.0 | 0.8 |
| (PBMCs)_resting | | | | |
| 93113_Mononuclear Cells | 7.6 | Lymph node | 0.7 | 0.9 |
| (PBMCs)_PWM | | | | |
| 93114_Mononuclear Cells | 8.7 | Colorectal | 1.0 | 0.6 |
| (PBMCs)_PHA-L | | | | |
| 93249_Ramos (B cell)_none | 21.0 | Stomach | 1.5 | 1.9 |
| 93250_Ramos (B cell)_ionomycin | 0.0 | Small intestine | 2.4 | 1.4 |
| 93349_B lymphocytes_PWM | 12.5 | Colon ca. SW 480 | 1.5 | 0.0 |
| 93350_B lymphoytes_CD40L and IL-4 | 0.0 | Colon ca.* (SW480 met) SW620 | 10.6 | 0.7 |
| 92665_EOL-1 | 0.0 | Colon ca. HT29 | 2.8 | 0.2 |
| (Eosinophil)_dbcAMP | | | | |
| differentiated | | | | |
| 93248_EOL-1 | 0.0 | Colon ca. HCT-116 | 16.8 | 1.1 |
| (Eosinophil)_dbcAMP/PMAionomycin | | | | |
| 93356_Dendritic Cells_none | 0.0 | Colon ca. CaCo-2 | 9.6 | 0.2 |
| 93355_Dendritic Cells_LPS 100 | 0.0 | 83219 CC Well to Mod Diff | 2.5 | 0.9 |
| ng/ml | | (ODO3566) | | |
| 93775_Dendritic Cells_anti-CD40 | 0.0 | Colon ca. HCC-2998 | 22.5 | 7.3 |
| 93774_Monocytes_resting | 20.0 | Gastric ca.* (liver met) NCI-N87 | 10.6 | 5.6 |
| 93776_Monocytes_LPS 50 ng/ml | 19.5 | Bladder | 4.0 | 1.4 |
| 93581_Macrophages_resting | 20.4 | Trachea | 1.1 | 0.2 |
| 93582_Macrophages_LPS 100 ng/ml | 7.5 | Kidney | 0.8 | 1.1 |
| 93098_HUVEC | 0.0 | Kidney (fetal) | 3.1 | 0.9 |
| (Endothelial)_none | | | | |
| 93099_HUVEC | 6.0 | Renal ca. 786-0 | 3.5 | 0.3 |
| (Endothelial)_starved | | | | |
| 93100_HUVEC (Endothelial) IL-1b | 10.2 | Renal ca. A498 | 5.2 | 1.2 |
| 93779_HUVEC (Endothelial)_IFN gamma | 0.0 | Renal ca. RXF 393 | 2.7 | 0.3 |
| 93102_HUVEC | 0.0 | Renal ca. ACHN | 4.3 | 0.6 |
| (Endothelial)_TNF alpha + IFN | | | | |
| gamma | | | | |
| 93101_HUVEC | 0.0 | Renal ca. UO-31 | 1.0 | 1.0 |
| (Endothelial)_TNF alpha + IL4 | | | | |
| 93781_HUVEC (Endothelial)_IL-11 | 0.0 | Renal ca. TK-10 | 9.7 | 2.2 |
| 93583_Lung Microvascular | 6.0 | Liver | 0.8 | 0.6 |
| Endothelial Cells_none | | | | |
| 93584_Lung Microvascular | 0.0 | Liver (fetal) | 4.4 | 2.2 |
| Endothelial Cells_TNFa (4 ng/ml) | | | | |
| and IL1b (1 ng/ml) | | | | |
| 92662_Microvascular Dermal | 8.7 | Liver ca. (hepatoblast) HepG2 | 4.7 | 0.5 |
| endothelium_none | | | | |
| 92663_Microsvasular Dermal | 0.0 | Lung | 0.1 | 0.2 |
| endothelium_TNFa (4 ng/ml) and | | | | |
| IL1b (1 ng/ml) | | | | |
| 93773_Bronchial epithelium_TNFa | 5.0 | Lung (fetal) | 0.1 | 0.5 |
| (4 ng/ml) and IL1b (1 ng/ml)** | | | | |
| 93347_Small Airway | 6.1 | Lung ca. (small cell) LX-1 | 15.6 | 1.6 |
| Epithelium_none | | | | |
| 93348_Small Airway | 6.6 | Lung ca. (small cell) NCI-H69 | 15.5 | 3.5 |
| Epithelium_TNFa (4 ng/ml) and | | | | |
| IL1b (1 ng/ml) | | | | |
| 92668_Coronery Artery | 0.0 | Lung ca. (s. cell var.) SHP-77 | 3.2 | 0.4 |
| SMC_resting | | | | |
| 92669_Coronery Artery | 0.0 | Lung ca. (large cell)NCI-H460 | 1.0 | 0.7 |
| SMC_TNFa (4 ng/ml) and IL1b (1 ng/ml) | | | | |
| 93107_astrocytes_resting | 0.0 | Lung ca. (non-sm. cell) A549 | 1.9 | 1.9 |
| 93108_astrocytes_TNFa (4 ng/ml) | 0.0 | Lung ca. (non-s. cell) NCI-H23 | 8.0 | 3.1 |
| and IL1b (1 ng/ml) | | | | |
| 92666_KU-812 (Basophil)_resting | 0.0 | Lung ca (non-s. cell) HOP-62 | 8.2 | 3.0 |
| 92667_KU-812 | 19.3 | Lung ca. (non-s. cl) NCI-H522 | 15.4 | 8.1 |
| (Basophil)_PMA/ionoycin | | | | |
| 93579_CCD1106 | 0.0 | Lung ca. (squam.) SW 900 | 7.9 | 0.0 |
| (Keratinocytes)_none | | | | |
| 93580_CCD1106 | 13.2 | Lung ca. (squam.) NCI-H596 | 14.6 | 0.7 |
| (Keratinocytes)_TNFa and IFNg** | | | | |
| 93791_Liver Cirrhosis | 79.6 | Mammary gland | 1.2 | 1.1 |
| 93792_Lupus Kidney | 6.7 | Breast ca.* (pl. effusion) MCF-7 | 7.1 | 1.1 |
| 93577_NCI-H292 | 0.0 | Breast ca.* (pl. ef) MDA-MB-231 | 7.8 | 0.7 |
| 93358_NCI-H292_IL-4 | 0.0 | Breast ca.* (pl. effusion) T47D | 10.1 | 0.0 |
| 93360_NCI-H292_IL-9 | 0.0 | Breast ca. BT-549 | 5.6 | 1.0 |
| 93359_NCI-H292_IL-13 | 15.5 | Breast ca. MDA-N | 14.2 | 0.0 |
| 93357_NCI-H292_IFN gamma | 7.4 | Ovary | 0.6 | 0.7 |
| 93777_HPAEC_- | 7.3 | Ovarian ca. OVCAR-3 | 8.9 | 3.3 |
| 93778_HPAEC_IL-1 beta/TNA alpha | 9.5 | Ovarian ca. OVCAR-4 | 3.1 | 0.7 |
| 93254_Normal Human Lung | 0.0 | Ovarian ca. OVCAR-5 | 9.9 | 3.5 |
| Fibroblast_none | | | | |
| 93253_Normal Human Lung | 0.0 | Ovarian ca. OVCAR-8 | 7.3 | 3.7 |
| Fibroblast_TNFa (4 ng/ml) and IL- | | | | |
| 1b (1 ng/ml) | | | | |
| 93257_Normal Human Lung | 0.0 | Ovarian ca. IGROV-1 | 4.0 | 0.8 |
| Fibroblast_IL-4 | | | | |
| 93256_Normal Human Lung | 0.0 | Ovarian ca.* (ascites) SK-OV-3 | 7.7 | 2.8 |
| Fibroblast_IL-9 | | | | |
| 93255_Normal Human Lung | 0.0 | Uterus | 0.3 | 0.0 |
| Fibroblast_IL-13 | | | | |
| 93258_Normal Human Lung | 0.0 | Plancenta | 4.2 | 2.4 |
| Fibroblast_IFN gamma | | | | |
| 93106_Dermal Fibroblasts | 0.0 | Prostate | 3.1 | 0.0 |
| CCD1070_resting | | | | |
| 93361_Dermal Fibroblasts | 7.6 | Prostate ca.* (bone met)PC-3 | 7.0 | 0.1 |
| CCD1070_TNF alpha 4 ng/ml | | | | |
| 93105_Dermal Fibroblasts | 0.0 | Testis | 55.5 | 21.3 |
| CCD1070_IL-1 beta 1 ng/ml | | | | |
| 93772_dermal fibroblast_IFN | 0.0 | Melanoma Hs688(A).T | 0.5 | 0.0 |
| gamma | | | | |
| 93771_dermal fibroblast_IL-4 | 0.0 | Melanoma* (met) Hs688(B).T | 0.6 | 0.0 |
| 93259_IBD Colitis 1** | 100.0 | Melanoma UACC-62 | 1.4 | 0.7 |
| 93260_IBD Colitis 2 | 0.0 | Melanoma M14 | 2.4 | 1.8 |
| 93261_IBD Crohns | 9.7 | Melanoma LOX IMVI | 0.8 | 0.0 |
| 735010_Colon_normal | 19.8 | Melanoma* (met) SK-MEL-5 | 3.6 | 0.2 |
| 735019_Lung_none | 15.7 | Adipose | 41.5 | 18.9 |
| 64028-1_Thymus_none | 24.1 | | | |
| 64030-1_Kidney_none | 9.0 | | | |

Panel 1.2 Summary: There is evidence for low level of expression of GPRCR8b across a number of the samples in panel 1.2. However, the predominant expression of this gene is localized to brain tissues, with highest levels being seen in the cerebral cortex. Lower levels are seen in the hippocampus, cerebellum, thalamus and amygdala. Prominent levels of expression are also seen in the pituitary, testis and adipose.

Panel 4D Summary: Expression of GPCR8b in Colitis 1 may be due to genomic contamination. There is a low level of expression in cirrhotic liver. Low level of expression is also seen in normal liver in panel 1.2. This suggests that GPCR8b and the protein it encodes may serve as markers for liver tissue.

### EXAMPLE 3F

### Expression Analysis of GPCR9 (SC80023385) Nucleic Acid

Expression of gene GPCR9 was assessed using the primer-probe set Ag1255, described in Table 16A. Results of the RTQ-PCR runs are shown in Table 16B.

TABLE 16A
Probe Name: Ag1255

| | | | | | Start |
|---|---|---|---|---|---|
| Primers | Sequences | | TM | Length | Position |
| Forward | 5′-CCAGTGGAGCTAAACATTTGTG-3′ | (SEQ ID NO:57) | 59.7 | 22 | 23 |
| Probe | TET-5′-TGCAGCCCTGTCTCTGTATAACTTCCG-3′- | (SEQ ID NO:58) | 69.2 | 27 | 47 |
| | TAMRA | | | | |
| Reverse | 5′-AGCAGCAGAGACCTGGAATAG-3′ | (SEQ. ID NO:59) | 58.7 | 21 | 84 |

TABLE 16B
Panels 1.2 and 4D

| | Rel. Expr., % | | Rel. Expr., % | Rel. Expr., % |
|---|---|---|---|---|
| Panel 1.2 | 1.2tm1421t_{—} | Panel 4D | 4Dtm2114t_{—} | 4Dtm2195t_{—} |
| Tissue Name | ag1255 | Tissue Name | ag1255 | ag1255 |
| Endothelial cells | 0.0 | 93768_Secondary Th1_anti- | 17.7 | 14.0 |
| | | CD28/anti-CD3 | | |
| Endothelial cells (treated) | 0.7 | 93769_Secondary Th2_anti- | 23.3 | 22.2 |
| | | CD28/anti-CD3 | | |
| Pancreas | 7.2 | 93770_Secondary Tr1_anti- | 20.2 | 20.0 |
| | | CD28/anti-CD3 | | |
| Pancreatic ca. CAPAN 2 | 12.3 | 93573_Secondary Th1_resting day | 32.8 | 19.5 |
| | | 4-6 in IL-2 | | |
| Adrenal Gland (new lot*) | 34.2 | 93572_Secondary Th2_resting day | 39.2 | 48.0 |
| | | 4-6 in IL-2 | | |
| Thyroid | 15.7 | 93571_Secondary Tr1 resting day | 22.2 | 24.1 |
| | | 4-6 in IL-2 | | |
| Salavary gland | 48.6 | 93568_primary Th1_anti- | 17.1 | 12.9 |
| | | CD28/anti-CD3 | | |
| Pituitary gland | 14.9 | 93569_primary Th2_anti- | 19.2 | 32.5 |
| | | CD28/anti-CD3 | | |
| Brain (fetal) | 1.7 | 93570_primary Tr1_anti-CD28/anti-CD3 | 39.0 | 32.5 |
| Brain (whole) | 27.5 | 93565_primary Th1_resting dy 4-6 | 86.5 | 100.0 |
| | | in IL-2 | | |
| Brain (amygdala) | 20.0 | 93566_primary Th2_resting dy 4-6 | 73.2 | 65.1 |
| | | in IL-2 | | |
| Brain (cerebellum) | 11.8 | 93567_primary Tr1_resting dy 4-6 | 79.6 | 83.5 |
| | | in IL-2 | | |
| Brain (hippocampus) | 29.5 | 93351_CD45RA CD4 | 5.5 | 3.5 |
| | | lymphocyte_anti-CD28/anti-CD3 | | |
| Brain (thalamus) | 34.6 | 93352_CD45RO CD4 | 14.4 | 17.7 |
| | | lymphocyte_anti-CD28/anti-CD3 | | |
| Cerebral Cortex | 24.3 | 93251_CD8 Lymphocytes_anti- | 11.8 | 25.3 |
| | | CD28/anti-CD3 | | |
| Spinal cord | 53.2 | 93353_chronic CD8 Lymphocytes | 14.1 | 11.1 |
| | | 2ry_resting dy 4-6 in IL-2 | | |
| CNS ca. (glio/astro) | 0.1 | 93574_chronic CD8 Lymphocytes | 21.3 | 14.0 |
| U87-MG | | 2ry_activated CD3/CD28 | | |
| CNS ca. (glio/astro) | 0.0 | 93354_CD4_none | 6.5 | 6.9 |
| U-118-MG | | | | |
| CNS ca. (astro) | 0.0 | 93252_Secondary | 100.0 | 91.4 |
| SW1783 | | Th1/Th2/Tr1_anti-CD95 CH11 | | |
| CNS ca.* (neuro; met) SK- | 0.7 | 93103_LAK cells_resting | 27.4 | 22.8 |
| N-AS | | | | |
| CNS ca. (astro) SF-539 | 0.6 | 93788_LAK cells_IL-2 | 33.7 | 32.5 |
| CNS ca. (astro) SNB-75 | 0.0 | 93787_LAK cells_IL-2 + IL-12 | 30.4 | 15.2 |
| CNS ca. (glio) SNB-19 | 0.7 | 93789_LAK cells_IL-2 + IFN | 41.8 | 25.7 |
| | | gamma | | |
| CNS ca. (glio) U251 | 0.0 | 93790_LAK cells_IL-2 + IL-18 | 33.0 | 15.6 |
| CNS ca. (glio) SF-295 | 0.0 | 93104_LAK cells_PMA/ionomycin | 4.2 | 0.6 |
| | | and IL-18 | | |
| Heart | 22.1 | 93578_NK Cells IL-2_resting | 34.2 | 38.4 |
| Skeletal Muscle (new lot*) | 1.5 | 93109_Mixed Lymphocyte | 31.6 | 32.1 |
| | | Reaction_Two Way MLR | | |
| Bone marrow | 5.8 | 93110_Mixed Lymphocyte | 7.6 | 6.7 |
| | | Reaction_Two Way MLR | | |
| Thymus | 20.2 | 93111_Mixed Lymphocyte | 8.4 | 4.1 |
| | | Reaction_Two Way MLR | | |
| Spleen | 44.4 | 93112_Mononuclear Cells | 1.9 | 0.3 |
| | | (PBMCs)_resting | | |
| Lymph node | 79.0 | 93113_Mononuclear Cells | 32.5 | 31.9 |
| | | (PBMCs)_PWM | | |
| Colorectal | 8.1 | 93114_Mononuclear Cells | 46.3 | 43.8 |
| | | (PBMCs)_PHA-L | | |
| Stomach | 100.0 | 93249_Ramos (B cell)_none | 9.1 | 13.5 |
| Small intestine | 98.6 | 93250_Ramos (B cell)_ionomycin | 24.0 | 13.9 |
| Colon ca. SW480 | 7.0 | 93349_B lymphocytes_PWM | 39.2 | 39.5 |
| Colon ca.* (SW480 | 6.4 | 93350_B lymphoytes_CD40L and | 40.6 | 49.7 |
| met)SW620 | | IL-4 | | |
| Colon ca. | 0.7 | 92665_EOL-1 | 4.8 | 3.8 |
| HT29 | | (Eosinophil)_dbcAMP differentiated | | |
| Colon ca. HCT-116 | 8.0 | 93248_EOL-1 | 2.1 | 0.6 |
| | | (Eosinophil)_dbcAMP/PMAionomycin | | |
| Colon ca. CaCo-2 | 0.2 | 93356_Dendritic Cells_none | 5.1 | 1.4 |
| 83219 CC Well to Mod | 11.3 | 93355_Dendritic Cells_LPS 100 | 0.0 | 0.0 |
| Diff (ODO3866) | | ng/ml | | |
| Colon ca. HCC-2998 | 18.7 | 93775_Dendritic Cells_anti-CD40 | 2.0 | 1.2 |
| Gastric ca.* (liver met) | 72.7 | 93774_Monocytes_resting | 2.9 | 1.5 |
| NCI-N87 | | | | |
| Bladder | 56.3 | 93776_Monocytes_LPS 50 ng/ml | 3.2 | 5.1 |
| Trachea | 9.8 | 93581_Macrophages_resting | 16.5 | 14.1 |
| Kidney | 3.5 | 93582_Macrophages_LPS 100 | 1.2 | 3.6 |
| | | ng/ml | | |
| Kidney (fetal) | 9.2 | 93098_HUVEC (Endothelial)_none | 0.3 | 0.0 |
| Renal ca. 786-0 | 0.0 | 93099_HUVEC | 0.0 | 0.6 |
| | | (Endothelial)_starved | | |
| Renal ca. A498 | 0.0 | 93100_HUVEC (Endothelial)_IL-1b | 0.0 | 0.0 |
| Renal ca. RXF 393 | 0.0 | 93779_HUVEC (Endothelial)_IFN | 0.0 | 1.0 |
| | | gamma | | |
| Renal ca. ACHN | 0.4 | 93102_HUVEC (Endothelial)_TNF | 0.0 | 0.0 |
| | | alpha + IFN gamma | | |
| Renal ca. UO-31 | 0.4 | 93101_HUVEC (Endothelial)_TNF | 0.0 | 0.0 |
| | | alpha + IL4 | | |
| Renal ca. TK-10 | 0.0 | 93781_HUVEC (Endothelial)_IL-11 | 0.0 | 0.0 |
| Liver | 16.8 | 93583_Lung Microvascular | 0.0 | 0.0 |
| | | Endothelial Cells_none | | |
| Liver (fetal) | 4.3 | 93584_Lung Microvascular | 0.0 | 0.0 |
| | | Endothelial Cells_TNFa (4 ng/ml) | | |
| | | and IL1b (1 ng/ml) | | |
| Liver ca. (hepatoblast) | 0.6 | 92662_Microvascular Dermal | 0.0 | 0.0 |
| HepG2 | | endothelium_none | | |
| Lung | 3.8 | 92663_Microsvasular Dermal | 0.0 | 0.0 |
| | | endothelium_TNFa (4 ng/ml) and | | |
| | | IL1b (1 ng/ml) | | |
| Lung (fetal) | 3.0 | 93773_Bronchial epithelium_TNFa | 19.9 | 12.0 |
| | | (4 ng/ml) and IL1b (1 ng/ml)** | | |
| Lung ca. (small cell) | 73.2 | 93347_Small Airway | 2.3 | 6.2 |
| LX-1 | | Epithelium_none | | |
| Lung ca. (small cell) NCI-H69 | 7.0 | 93348_Small Airway | 33.4 | 33.4 |
| | | Epithelium_TNFa (4 ng/ml) and | | |
| | | IL1b (1 ng/ml) | | |
| Lung ca. (s. cell var.) SHP-77 | 0.0 | 92668_Coronery Artery | 0.0 | 0.0 |
| | | SMC_resting | | |
| Lung ca. (large cell) NCI-H460 | 0.3 | 92669_Coronery Artery | 0.0 | 0.5 |
| | | SMC_TNFa (4 ng/ml) and ILlb (1 ng/ml) | | |
| Lung ca. (non-sm. cell) A549 | 4.3 | 93107_astrocytes_resting | 0.0 | 0.6 |
| Lung ca. (non-s. cell) NCI-H23 | 0.0 | 93108_astrocytes_TNFa (4 ng/ml) | 0.0 | 0.0 |
| | | and IL1b (1 ng/ml) | | |
| Lung ca (non-s. cell) HOP-62 | 3.7 | 92666_KU-812 (Basophil)_resting | 27.2 | 23.5 |
| Lung ca. (non-s. cl) NCI-H522 | 0.5 | 92667_KU-812 | 80.1 | 78.5 |
| | | (Basophil)_PMA/ionoycin | | |
| Lung ca. (squam.) SW 900 | 0.0 | 93579_CCD1106 | 10.7 | 9.2 |
| | | (Keratinocytes)_none | | |
| Lung ca. (squam.) NCI-H596 | 2.0 | 93580_CCD1106 | 83.5 | 71.2 |
| | | (Keratinocytes)_TNF and IFNg** | | |
| Mammary gland | 8.8 | 93791_Liver Cirrhosis | 5.2 | 3.7 |
| Breast ca.* (pl. effusion) | 0.1 | 93792_Lupus Kidney | 3.7 | 1.9 |
| MCF-7 | | | | |
| Breast ca.* (pl. ef) MDA- | 0.4 | 93577_NCI-H292 | 38.4 | 47.6 |
| MB-231 | | | | |
| Breast ca.* (pl. effusion) | 0.8 | 93358_NCI-H292_IL-4 | 40.9 | 28.7 |
| T47D | | | | |
| Breast ca. BT-549 | 0.2 | 93360_NCI-H292_IL-9 | 38.2 | 29.5 |
| Breast ca. MDA-N | 0.3 | 93359_NCI-H292_IL-13 | 38.7 | 27.7 |
| Ovary | 1.1 | 93357_NCI-H292_IFN gamma | 10.7 | 15.9 |
| Ovarian ca. OVCAR-3 | 1.7 | 93777_HPAEC_- | 0.4 | 0.0 |
| Ovarian ca. OVCAR-4 | 0.2 | 93778_HPAEC_IL-I beta/TNA | 1.2 | 0.0 |
| | | alpha | | |
| Ovarian ca. OVCAR-5 | 30.6 | 93254_Normal Human Lung | 0.6 | 0.0 |
| | | Fibroblast_none | | |
| Ovarian ca. OVCAR-8 | 5.6 | 93253_Normal Human Lung | 0.6 | 0.0 |
| | | Fibroblast_TNFa (4 ng/ml) and IL-1b | | |
| | | (1 ng/ml) | | |
| Ovarian ca. | 0.3 | 93257_Normal Human Lung | 0.0 | 0.0 |
| IGROV-1 | | Fibroblast_IL-4 | | |
| Ovarian ca.* (ascites) | 1.0 | 93256_Normal Human Lung | 0.0 | 0.0 |
| SKOV-3 | | Fibroblast_IL-9 | | |
| Uterus | 2.5 | 93255_Normal Human Lung | 0.0 | 0.0 |
| | | Fibroblast_IL-13 | | |
| Plancenta | 20.7 | 93258_Normal Human Lung | 0.0 | 0.0 |
| | | Fibroblast_IFN gamma | | |
| Prostate | 8.4 | 93106_Dermal Fibroblasts | 0.0 | 0.0 |
| | | CCD1070_resting | | |
| Prostate ca.* (bone | 0.5 | 93361_Dermal Fibroblasts | 36.1 | 51.0 |
| met)PC-3 | | CCD1070_TNF alpha 4 ng/ml | | |
| Testis | 35.6 | 93105_Dermal Fibroblasts | 0.0 | 0.0 |
| | | CCD1070_IL-1 beta 1 ng/ml | | |
| Melanoma | 0.0 | 93772_dermal fibroblast_IFN | 0.0 | 0.0 |
| Hs688(A).T | | gamma | | |
| Melanoma* (met) | 0.0 | 93771_dermal fibroblast_IL-4 | 0.0 | 1.3 |
| Hs688(B).T | | | | |
| Melanoma UACC-62 | 0.0 | 93259_IBD Colitis 1** | 9.3 | 7.3 |
| Melanoma M14 | 0.2 | 93260_IBD Colitis 2 | 4.6 | 3.1 |
| Melanoma LOX IMVI | 0.0 | 93261_IBD Crohns | 2.0 | 1.3 |
| Melanoma* (met) SK-MEL-5 | 0.0 | 735010_Colon_normal | 30.1 | 28.3 |
| Adipose | 47.0 | 735019_Lung_none | 5.7 | 1.1 |
| | | 64028-1_Thymus_none | 4.4 | 9.9 |
| | | 64030-1_Kidney_none | 19.3 | 15.5 |

Panel 1.2 Summary: GPCR9 shows expression in a number of samples across panel 1.2. In particular there is a cluster of expression associated with normal brain and brain cancer cell lines. In addition, the highest levels of expressions are associated with the upper gastrointestinal (GI) tract (stomach, small intestine) when compared to the lower GI tract (colorectal). This might indicate a role for SC80023385 in digestion. Significantly high levels of this transcript are also seen in the lymph node, spleen, thymus as well as in heart, bladder, testis and placenta.

Panel 4D Summary: This gene GPCR9, is upregulated in several tissues and cell types after activation including lymphocytes, keratinocytes, dermal fibroblasts, small airway epithelium and T cells. The putative GPCR encoded for by this transcript may function in the inflammatory process by promoting leukocyte extravasation or initiating a signaling cascade that could result in the release of immunomodulatory products such as cytokines. Antibody or small molecule therapeutics designed against the protein encoded by this transcript may reduce or inhibit inflammation due to psoriasis, delayed type hypersensitivity, asthma, or emphysema.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NO:2, 4, 6, 8, 10, 12, 14, 16, 18, 20, 22, 24 and/or 26;
(b) a variant of a mature form of an amino acid sequence selected from the group consisting of SEQ ID NO:2, 4, 6, 8, 10, 12, 14, 16, 18, 20, 22, 24 and/or 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 12, 14, 16, 18, 20, 22, 24 and/or 26; and
(d) a variant of an amino acid sequence selected from the group consisting of SEQ ID NO:2, 4, 6, 8, 10, 12, 14, 16, 18, 20, 22, 24 and/or 26, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NO:2, 4, 6, 8, 10, 12, 14, 16, 18, 20, 22, 24 and/or 26.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:2, 4, 6, 8, 10, 12, 14, 16, 18, 20, 22, 24 and/or 26.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO:2, 4, 6, 8, 10, 12, 14, 16, 18, 20, 22, 24 and/or 26;
(b) a variant of a mature form of an amino acid sequence selected from the group consisting of SEQ ID NO:2, 4, 6, 8, 10, 12, 14, 16, 18, 20, 22, 24 and/or 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 12, 14, 16, 18, 20, 22, 24 and/or 26;
(d) a variant of an amino acid sequence selected from the group consisting of SEQ ID NO:2, 4, 6, 8, 10, 12, 14, 16, 18, 20, 22, 24 and/or 26, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO:2, 4, 6, 8, 10, 12, 14, 16, 18, 20, 22, 24 and/or 26, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:1 3, 5, 7, 9, 11, 13, 15, 17, 19, 21, 23 and 25.

9. The nucleic acid molecule of claim 5, wherein said nucleic acid molecule comprises a nucleotide sequence selected from the group consisting of:
(a) a nucleotide sequence selected from the group consisting of SEQ ID NO:1 3, 5, 7, 9, 11, 13, 15, 17, 19, 21, 23 and 25;
(b) a nucleotide sequence differing by one or more nucleotides from a nucleotide sequence selected from the group consisting of SEQ ID NO:1 3, 5, 7, 9, 11, 13, 15, 17, 19, 21, 23 and 25, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NO:1 3, 5, 7, 9, 11, 13, 15, 17, 19, 21, 23 and 25, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a GPCRX-associated disorder, said method comprising administering to a subject in which such treatment or prevention is desired the polypeptide of claim 1 in an amount sufficient to treat or prevent said GPCRX-associated disorder in said subject.

27. The method of claim 26, wherein said subject is a human.

28. A method of treating or preventing a GPCRX-associated disorder, said method comprising administering to a subject in which such treatment or prevention is desired the nucleic acid of claim 5 in an amount sufficient to treat or prevent said GPCRX-associated disorder in said subject.

29. The method of claim 28, wherein said subject is a human.

30. A method of treating or preventing a GPCRX-associated disorder, said method comprising administering to a subject in which such treatment or prevention is desired the antibody of claim 15 in an amount sufficient to treat or prevent said GPCRX-associated disorder in said subject.

31. The method of claim 30, wherein the subject is a human.

32. A pharmaceutical composition comprising the polypeptide of claim 1 and a pharmaceutically-acceptable carrier.

33. A pharmaceutical composition comprising the nucleic acid molecule of claim 5 and a pharmaceutically-acceptable carrier.

34. A pharmaceutical composition comprising the antibody of claim 15 and a pharmaceutically-acceptable carrier.

35. A kit comprising in one or more containers, the pharmaceutical composition of claim 32.

36. A kit comprising in one or more containers, the pharmaceutical composition of claim 33.

37. A kit comprising in one or more containers, the pharmaceutical composition of claim 34.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO:2, 4, 6, 8, 10, 12, 14, 16 18, 20, 22, 24 and/or 26, or a biologically active fragment thereof.

41. A method of treating a pathological state in a mammal, the method comprising administering to the mammal the antibody of claim 15 in an amount sufficient to alleviate the pathological state.

42. A method for the screening of a candidate substance interacting with an olfactory receptor polypeptide selected from the group consisting of SEQ ID NO:2, 4, 6, 8, 10, 12, 14, 16 18, 20, 22, 24 and/or 26, or fragments or variants thereof, which comprises the following steps:
a) providing a polypeptide selected from the group consisting of the sequences of SEQ ID NO:2, 4, 6, 8, 10, 12, 14, 16 18, 20, 22, 24 and/or 26, or a peptide fragment or a variant thereof;
b) obtaining a candidate substance;
c) bringing into contact said polypeptide with said candidate substance; and
d) detecting the complexes formed between said polypeptide and said candidate substance.

43. A method for the screening of ligand molecules interacting with an olfactory receptor polypeptide selected from the group consisting of SEQ ID NO:2, 4, 6, 8, 10, 12, 14, 16 18, 20, 22, 24 and/or 26, wherein said method comprises:
a) providing a recombinant eukaryotic host cell containing a nucleic acid encoding a polypeptide selected from the group consisting of the polypeptides comprising the amino acid sequences SEQ ID NO:2, 4, 6, 8, 10, 12, 14, 16 18, 20, 22, 24 and/or 26;
b) preparing membrane extracts of said recombinant eukaryotic host cell;
c) bringing into contact the membrane extracts prepared at step b) with a selected ligand molecule; and
d) detecting the production level of second messengers metabolites.

44. A method for the screening of ligand molecules interacting with an olfactory receptor polypeptide selected from the group consisting of SEQ ID NO:2, 4, 6, 8, 10, 12, 14, 16 18, 20, 22, 24 and/or 26, wherein said method comprises:
a) providing an adenovirus containing a nucleic acid encoding a polypeptide selected from the group consisting of polypeptides comprising the amino acid sequences SEQ ID NO:2, 4, 6, 8, 10, 12, 14, 16 18, 20, 22, 24 and/or 26;
b) infecting an olfactory epithelium with said adenovirus;
c) bringing into contact the olfactory epithelium b) with a selected ligand molecule; and
d) detecting the increase of the response to said ligand molecule.
